(19) United States
(12) Patent Application Publication
(10) Pub. No.: US 2025/0034550 A1
(43) Pub. Date: Jan. 30, 2025

(54) COMPOSITIONS AND METHODS FOR POLYNUCLEOTIDE ASSEMBLY

(71) Applicant: Guangzhou Chengyuan Bioimmunology Technology Co., Ltd., Huangpu District, Guangzhou Guangdong (CN)

(72) Inventor: Xi CHEN, Newton, MA (US)

(21) Appl. No.: 18/713,058

(22) PCT Filed: Nov. 22, 2022
(86) PCT No.: PCT/US2022/050685

§ 371 Date: May 23, 2024

Related U.S. Application Data
(60) Provisional application No. 63/282,845, filed on Nov. 24, 2021.
(60) Provisional application No. 63/305,488, filed on Feb. 1, 2022.

(51) Int. Cl. C12N 15/10; C12P 19/34

(52) U.S. Cl. CPC C12N 15/1031; C12P 19/34

(57) ABSTRACT

Provided herein are compositions and methods for assembling (e.g., synthesizing) different nucleic acid sequences in a mixture. Each of the different nucleic acid sequences can be assembled from two or more nucleic acid fragments containing connector sequences (e.g., Zip sequences described herein) for specifically linking the two or more nucleic acid fragments.

### CROSS-REFERENCE

This application claims the benefit of U.S. Provisional Patent Application No. 63/282,845, filed on Nov. 24, 2021, and U.S. Provisional Patent Application No. 63/305,488, filed on Feb. 1, 2022, the entire content of each of which is incorporated herein by reference.

### BACKGROUND OF THE INVENTION

Gene synthesis is a broadly enabling technology for life science research and health care. While the cost of DNA sequencing has dropped by five orders of magnitude in the past decade, DNA synthesis remains expensive for many applications. Although DNA microarrays have decreased the cost of oligonucleotide synthesis, the use of array synthesized oligos in practice is limited by short synthesis lengths, high synthesis error rates, low yield and the challenges of assembling long constructs from complex pools.

### SUMMARY OF THE INVENTION

Recognized herein is a need for a cheap, controlled, and high quality, high throughput way to assemble or synthesize a pool of long polynucleotides from relatively short oligo fragments. The pool of polynucleotides of interest can be assembled or synthesized from various fragments in a same mixture without non-specific linkages. The present disclosure provides compositions and methods for assembling or synthesizing polynucleotides of interest using a large number (e.g., hundreds, thousands or more) of designed connector sequences (also referred to as Zips in this disclosure). The polynucleotides of interest assembled or synthesized herein can be any sequences of interest. The polynucleotides of interest assembled or synthesized herein can be a functional genetic element not limited to a gene or a protein-coding sequence.

In an aspect, the present disclosure provides a method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBⱼ sequence of a jth polynucleotide when i≠j;
(c) contacting the first mixture and the second mixture, thereby generating a third mixture of at least n polynucleotides, wherein an ith polynucleotide comprises a SeqAᵢ sequence, a SeqBᵢ sequence and a ZipABᵢ sequence, wherein the ZipABᵢ sequence is different from a ZipABⱼ sequence of a jth polynucleotide when i≠j;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(e) contacting the third mixture and the fourth mixture, thereby generating a fifth mixture of at least n polynucleotides, wherein an ith polynucleotide comprises the Seqᵢ sequence comprising the SeqAᵢ sequence, the SeqBᵢ sequence and the SeqCᵢ sequence.

In some embodiments, generating the third mixture of at least n polynucleotides comprises specifically linking, for each i, the ZipAᵢ sequence and the ZipBᵢ sequence. In some embodiments, generating the fifth mixture of at least n polynucleotides comprises specifically linking, for each i, the ZipCᵢ sequence and the ZipABᵢ sequence.

In another aspect, the present disclosure provides a method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j;
(c) generating a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, and wherein generating comprises contacting the first mixture and the second mixture such that, for each i, the ZipAᵢ sequence specifically links to the ZipBᵢ sequence;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(e) generating a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein generating comprises contacting the third mixture and the fourth mixture such that, for each i, the ZipCᵢ sequence specifically links to the ZipABᵢ sequence. In some embodiments, for each i, the ZipAᵢ sequence and the ZipBᵢ sequence are a same nucleic acid sequence or are complementary. In some embodiments, for each i, the ZipABᵢ sequence is a same nucleic acid sequence as the ZipAᵢ sequence or the ZipBᵢ sequence. In some embodiments, for each i, the ZipAᵢ sequence and the ZipBᵢ sequence are different nucleic acid sequences. In some embodiments, for each i, the ZipABᵢ sequence, the ZipAᵢ sequence, and the ZipBᵢ sequence are a same nucleic acid sequence. In some embodiments, for each i, the ZipABᵢ sequence is a different nucleic acid sequence from the ZipAᵢ sequence or the ZipBᵢ sequence. In some embodiments, the ZipABᵢ sequence, the ZipAᵢ sequence, and the ZipBᵢ sequence are different nucleic acid sequences.

In another aspect, the present disclosure provides a method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of a jth polynucleotide when i≠j; (
b) providing a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j;
(c) generating a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, wherein the ZipABᵢ sequence is a same nucleic acid sequence as the ZipAᵢ sequence or the ZipBᵢ sequence, and wherein generating comprises contacting the first mixture and the second mixture such that, for each i, the ZipAᵢ sequence links to the ZipBᵢ sequence;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCᵢ sequence of a jth polynucleotide when i≠j; and
(e) generating a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein generating comprises contacting the third mixture and the fourth mixture such that, for each i, the ZipCᵢ sequence links to the ZipABᵢ sequence. In some embodiments, for each i, the ZipAᵢ sequence specifically links to the ZipBᵢ sequence. In some embodiments, for each i, the ZipCᵢ sequence specifically links to the ZipABᵢ sequence. In some embodiments, for each i, the ZipAᵢ sequence and the ZipBᵢ sequence are a same nucleic acid sequence or are complementary. In some embodiments, for each i, the ZipAᵢ sequence and the ZipBᵢ sequence are different nucleic acid sequences. In some embodiments, for each i, the ZipABᵢ sequence, the ZipAᵢ sequence, and the ZipBᵢ sequence are a same nucleic acid sequence.

In some embodiments, the SeqAᵢ sequence, the SeqBᵢ sequence and the SeqCᵢ sequence of the Seqᵢ sequence are linked seamlessly (e.g., without any intervening sequences). In some embodiments, the Seqᵢ sequence comprises the SeqAᵢ sequence and the SeqBᵢ sequence without an intervening sequence in between the SeqAᵢ sequence and the SeqBᵢ sequence.

In some embodiments, the Seqᵢ sequence comprises the SeqBᵢ sequence and the SeqCᵢ sequence without an intervening sequence in between the SeqBᵢ sequence and the SeqCᵢ sequence. In some embodiments, the Seqᵢ sequence with an intervening sequence in between the SeqAᵢ sequence and the SeqBᵢ sequence or the SeqBᵢ sequence and the SeqCᵢ sequence is not a functional genetic element. In some embodiments, the Seqᵢ sequence comprises the SeqAᵢ sequence, the SeqBᵢ sequence and the SeqCᵢ sequence sequentially from the 5′ end to the 3′ end.

In some embodiments, for each i, the ZipAᵢ sequence and the ZipBᵢ sequence are connector sequences for specifically linking the SeqAᵢ sequence and the SeqBᵢ sequence. In some embodiments, for each i, the ZipCᵢ sequence and the ZipABᵢ sequence are connector sequences for specifically linking the SeqCᵢ sequence and a sequence comprising the SeqAᵢ sequence and the SeqBᵢ sequence. In some embodiments, for each i, the ZipAᵢ sequence and the ZipBᵢ sequence are a same nucleic acid sequence. In some embodiments, for each i, the ZipAᵢ sequence and the ZipBᵢ sequence are complementary. In some embodiments, for each i, the ZipAᵢ sequence and the ZipBᵢ sequence are different nucleic acid sequences. In some embodiments, for each i, the ZipABᵢ sequence is a same nucleic acid sequence as the ZipAᵢ sequence or the ZipBᵢ sequence. In some embodiments, for each i, the ZipABᵢ sequence, the ZipAᵢ sequence, and the ZipBᵢ sequence are a same nucleic acid sequence. In some embodiments, for each i, the ZipABᵢ sequence is a different nucleic acid sequence from the ZipAᵢ sequence or the ZipBᵢ sequence.

In some embodiments, the ZipABᵢ sequence, the ZipAᵢ sequence, and the ZipBᵢ sequence are different nucleic acid sequences. In some embodiments, the ZipCᵢ sequence and the ZipABᵢ sequence are a same nucleic acid sequence. In some embodiments, the ZipCᵢ sequence and the ZipABᵢ sequence are complementary. In some embodiments, the ZipCᵢ sequence and the ZipABᵢ sequence are different nucleic acid sequences. In some embodiments, for each i, the ZipAᵢ sequence, the ZipBᵢ sequence, the ZipABᵢ sequence, or the ZipCᵢ sequence is from 5 nucleotides to 200 nucleotides in length. In some embodiments, for each i, the SeqAᵢ sequence, the SeqBᵢ sequence, or the SeqCᵢ sequence is from 5 nucleotides to 5,000 nucleotides in length.

In some embodiments, generating the third mixture of at least n polynucleotides comprises linking, for each i, the ZipAᵢ sequence and the ZipBᵢ sequence. In some embodiments, for each i, the ZipAᵢ sequence hybridizes to the ZipBᵢ sequence. In some embodiments, the method further comprises extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence. In some embodiments, for each i, the ith polynucleotide of the third mixture further comprises an Operator sequence that is a primer binding site. In some embodiments, the Operator sequence is a same sequence among the third mixture of at least n polynucleotides. In some embodiments, the method further comprises removing the Operator sequence. In some embodiments, removing comprises using an enzyme to degrade the Operator sequence.

In some embodiments, the method further comprises circularizing the ith polynucleotide comprising the SeqAᵢ sequence, the SeqBᵢ sequence and the ZipABᵢ sequence to generate a circularized polynucleotide. In some embodiments, circularizing the ith polynucleotide comprising the SeqAᵢ sequence, the SeqBᵢ sequence and the ZipABᵢ sequence comprises circularizing the ith polynucleotide by a ligase. In some embodiments, the method further comprises linearizing the circularized polynucleotide. In some embodiments, linearizing the circularized product comprises cutting the circularized polynucleotide or amplifying the circularized polynucleotide using polymerase chain reaction (PCR). In some embodiments, linearizing the circularized product such that the ZipABᵢ sequence is not flanked by the SeqAᵢ sequence and the SeqBᵢ sequence. In some embodiments, the method further comprises exposing the ZipABᵢ sequence on a terminus of the ith polynucleotide comprising the SeqAᵢ sequence, the SeqBᵢ sequence and the ZipABᵢ sequence. In some embodiments, the ZipABᵢ sequence is not flanked by the SeqAᵢ sequence and the SeqBᵢ sequence. In some embodiments, the ZipABᵢ sequence is at a terminus of the ith polynucleotide comprises the SeqAᵢ sequence, the SeqBᵢ sequence and the ZipABᵢ sequence.

In some embodiments, generating the fifth mixture of at least n polynucleotides comprises linking, for each i, the ZipCᵢ sequence and the ZipABᵢ sequence. In some embodiments, linking comprising hybridizing the ZipCᵢ sequence and the ZipABᵢ sequence. In some embodiments, the method further comprises repeating operations above for the third mixture of n polynucleotides and the fourth mixture of n polynucleotides, thereby generating the fifth mixture of n polynucleotides. In some embodiments, the method further comprises removing the ZipCᵢ sequence and the ZipABᵢ sequence, thereby generating the ith polynucleotide comprises the Seqᵢ sequence comprising the SeqAᵢ sequence, the SeqBᵢ sequence and the SeqCᵢ sequence.

In some embodiments, the method further comprises, prior to (a) or (b), providing a pool of polynucleotides comprising the at least n polynucleotides of the first mixture, the at least n polynucleotides of the second mixture, and/or the at least n polynucleotides of the fourth mixture. In some embodiments, the method further comprises amplifying the at least n polynucleotides of the first mixture, the at least n polynucleotides of the second mixture, and/or the at least n polynucleotides of the fourth mixture from the pool to generate double-stranded polynucleotides. In some embodiments, only the at least n polynucleotides of the first mixture, the at least n polynucleotides of the second mixture, or the at least n polynucleotides of the fourth mixture are amplified from the pool. In some embodiments, the method further comprises removing an Operator sequence from the double-stranded polynucleotides, and wherein the Operator sequence is a primer binding site. In some embodiments, degrading one strand of the double-stranded polynucleotides to generate the at least n polynucleotides of the first mixture, the at least n polynucleotides of the second mixture, and/or the at least n polynucleotides of the fourth mixture.

In another aspect, the present disclosure provides a method of synthesizing a plurality of n different polynucleotides (where n is equal to or greater than 2) in a same mixture, wherein
(i) an ith polynucleotide of the plurality comprises a Seqi sequence (where i=1 ton), and
(ii) the Seqi sequence comprises a SeqAi sequence, a SeqBi sequence and a SeqCi sequence, the method comprising:
(a) providing a mixture comprising a first subpopulation of at least n polynucleotides, a second subpopulation of at least n polynucleotides, and a third subpopulation of at least n polynucleotides, wherein
(1) in the first subpopulation, an ith polynucleotide comprises a SeqAᵢ sequence and a ZipAᵢ sequence, and wherein the ZipAᵢ sequence is different from a ZipAⱼ sequence of a jth polynucleotide when i≠j,
(2) in the second subpopulation, an ith polynucleotide comprises a SeqBᵢ sequence and a ZipBᵢ sequence, and wherein the ZipBᵢ sequence is different from a ZipBᵢ sequence of a jth polynucleotide when i≠j, and
(3) in the third subpopulation, an ith polynucleotide comprises a SeqCᵢ sequence and a ZipCᵢ sequence, and wherein the ZipCᵢ sequence is different from a ZipCⱼ sequence of a jth polynucleotide when i≠j; and
(b) generating a plurality of n polynucleotides, wherein an ith polynucleotide of the plurality comprises the Seqᵢ sequence comprising the SeqAᵢ sequence, the SeqBᵢ sequence and the SeqCᵢ sequence, wherein generating comprises specifically linking the SeqAᵢ sequence, the SeqBᵢ sequence and the SeqCᵢ sequence.

In some embodiments, in the second subpopulation, the ZipBᵢ sequence is a ZipB1ᵢ sequence, and the ith polynucleotide further comprises a ZipB2ᵢ sequence. In some embodiments, the SeqBᵢ sequence is located in between the ZipB1ᵢ sequence and the ZipB2ᵢ sequence. In some embodiments, the ZipB1ᵢ sequence is located in between the SeqBᵢ sequence and the ZipB2ᵢ sequence. In some embodiments, the ZipB2ᵢ sequence is located in between the SeqBᵢ sequence and the ZipB1ᵢ sequence.

In some embodiments, the SeqAᵢ sequence, the SeqBᵢ sequence and the SeqCᵢ sequence of the Seqᵢ sequence are linked seamlessly. In some embodiments, the Seq, sequence comprises the SeqAᵢ sequence and the SeqBᵢ sequence without an intervening sequence in between the SeqAᵢ sequence and the SeqBᵢ sequence. In some embodiments, the Seqᵢ sequence comprises the SeqBᵢ sequence and the SeqCᵢ sequence without an intervening sequence in between the SeqBᵢ sequence and the SeqCᵢ sequence. In some embodiments, the Seqᵢ sequence comprises the SeqAᵢ sequence, the SeqBᵢ sequence and the SeqCᵢ sequence sequentially from the 5′ end to the 3′ end.

In some embodiments, the ZipAᵢ sequence and the ZipBᵢ sequence are connector sequences for specifically linking the SeqAᵢ sequence and the SeqBᵢ sequence. In some embodiments, the ZipB2ᵢ sequence and the ZipCᵢ sequence are connector sequences for specifically linking the SeqBᵢ sequence and the SeqCᵢ sequence. In some embodiments, for each i, the ZipAᵢ sequence and the ZipBᵢ sequence are a same nucleic acid sequence. In some embodiments, for each i, the ZipAᵢ sequence and the ZipBᵢ sequence are complementary. In some embodiments, for each i, the ZipAᵢ sequence and the ZipBᵢ sequence are different nucleic acid sequences. In some embodiments, for each i, the ZipB2ᵢ sequence and the ZipCᵢ sequence are a same nucleic acid sequence. In some embodiments, for each i, the ZipB2ᵢ sequence and the ZipCᵢ sequence are complementary. In some embodiments, for each i, the ZipB2ᵢ sequence and the ZipCᵢ sequence are different nucleic acid sequences. In some embodiments, for each i, the ZipAᵢ sequence, the ZipBᵢ sequence, the ZipB1ᵢ sequence, the ZipB2ᵢ sequence, or the ZipCᵢ sequence is from 5 nucleotides to 200 nucleotides in length. In some embodiments, for each i, the SeqAᵢ sequence, the SeqBᵢ sequence, or the SeqCᵢ sequence is from 5 nucleotides to 5,000 nucleotides in length.

In some embodiments, the method further comprises specifically linking (i) the ZipAᵢ sequence and the ZipB1ᵢ sequence, and/or (ii) the ZipB2ᵢ sequence and the ZipCᵢ sequence. In some embodiments, linking comprising hybridizing (i) the ZipAᵢ sequence and the ZipB1ᵢ sequence, and/or (ii) the ZipB2ᵢ sequence and the ZipCᵢ sequence.

In some embodiments, the method further comprises generating a plurality of intermediate products, wherein an ith intermediate product of the plurality comprises the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence.

In some embodiments, the ith intermediate product of the plurality comprises the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence sequentially from 5′ end to 3′ end.

In some embodiments, the method further comprises removing the ZipAᵢ sequence (or the ZipB1ᵢ sequence) and the ZipCᵢ sequence (or the ZipB2ᵢ sequence), thereby generating the Seqᵢ sequence comprising the SeqAᵢ sequence, the SeqBᵢ sequence and the SeqCᵢ sequence without any intervening sequence. In some embodiments, removing comprises using a DNA tweezer. In some embodiments, using the DNA tweezer comprises degrading one strand of the ZipAᵢ sequence or the ZipCᵢ sequence region, and using a staple strand to hybridize with regions flanking the ZipAᵢ sequence or the ZipCᵢ sequence on the complementary strand to bring the SeqAᵢ sequence, the SeqBᵢ sequence and the SeqCᵢ sequence region in close proximity for ligation.

In some embodiments, concatenation of the SeqAᵢ sequence and the SeqBᵢ sequence without an intervening sequence is a functional genetic element. In some embodiments, concatenation of the SeqAᵢ sequence, the SeqBᵢ sequence and the SeqCᵢ sequence without any intervening sequence is a functional genetic element. In some embodiments, the functional genetic element is a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, a primer-extension gRNA for prime editing, or any combination thereof. In some embodiments, the functional genetic element does not comprise a sequence that is identical to the ZipAᵢ sequence, the ZipBᵢ sequence, the ZipABᵢ sequence or the ZipCᵢ sequence. In some embodiments, for each i, the SeqAᵢ sequence, the SeqBᵢ sequence, and/or the SeqCᵢ sequence are uniquely or specifically linked. In some embodiments, a plurality of at least 2, 10, 20, 30, 40, 50, 60, 70, 80, 90, 100, 500, 1,000, 5,000 or more polynucleotides are synthesized. In some embodiments, each polynucleotide of the plurality synthesized is from about 15 to about 15,000 nucleotides in length.

In another aspect, the present disclosure provides a method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence and a SeqBᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j; and
(c) contacting the first mixture and the second mixture, thereby generating a third mixture of a plurality of n polynucleotides, wherein an ith polynucleotide comprises a ZipABᵢ sequence, a SeqAᵢ sequence and a SeqBᵢ sequence sequentially from 5′ end to 3′ end, and wherein the ZipABᵢ sequence is different from a ZipABᵢ sequence of a jth polynucleotide when i≠j;
wherein the SeqAᵢ sequence and the SeqBᵢ sequence are linked without an intervening sequence.

In some embodiments, for each i, the ZipAᵢ sequence and the ZipBᵢ sequence are connector sequences for specifically linking the SeqAᵢ sequence and the SeqBᵢ sequence. In some embodiments, generating the third mixture in (c) comprises specifically linking, for each i, the ZipAᵢ sequence and the ZipBᵢ sequence. In some embodiments, linking comprising hybridizing, for each i, the ZipAᵢ sequence and the ZipBᵢ sequence. In some embodiments, the method further comprises extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence. In some embodiments, the method further comprises generating an intermediate product comprising the SeqBᵢ sequence, the ZipABᵢ sequence and the SeqAᵢ sequence sequentially from 5′ end to 3′ end.

In some embodiments, the method further comprises contacting the third mixture with a fourth mixture of at least n polynucleotides, wherein an ith polynucleotide of the fourth mixture comprises a SeqCᵢ sequence and a ZipCᵢ sequence, and wherein the ZipCᵢ sequence is different from a ZipCᵢ sequence when i≠j.

In some embodiments, the method further comprises generating a fifth mixture of at least n polynucleotides, wherein an ith polynucleotide comprises the Seqᵢ sequence comprising the SeqAᵢ sequence and the SeqBᵢ sequence and further comprising the SeqCᵢ sequence.

In some embodiments, the SeqBᵢ sequence and the SeqCᵢ sequence are linked without an intervening sequence.

In another aspect, the present disclosure provides a method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAᵢ (where j=1 to n) sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j;
(c) contacting the first mixture and the second mixture to generate a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, and wherein, for each i, the ZipAᵢ sequence specifically links to the ZipBᵢ sequence;
(d) optionally, within the third mixture, for each i, extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence;
(e) optionally, within the third mixture, removing a sequence segment from 3′ and/or 5′ end of the ith polynucleotide;
(f) optionally, within the third mixture, circularizing the ith polynucleotide comprising the SeqAᵢ sequence, the SeqBᵢ sequence and the ZipABᵢ sequence to generate a circularized polynucleotide;
(g) optionally, within the third mixture, linearizing the circularized polynucleotide such that, for each i, the ZipABᵢ sequence is not flanked by the SeqAᵢ sequence and the SeqBᵢ sequence;
(h) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(i) contacting the third mixture and the fourth mixture to generate a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein, for each i, the ZipCᵢ sequence specifically links to the ZipABᵢ sequence.

In some embodiments, for each i, the ZipAᵢ sequence and the ZipBᵢ sequence are a same nucleic acid sequence. In some embodiments, for each i, the ZipAᵢ sequence and the ZipBᵢ sequence are complementary. In some embodiments, for each i, the ZipABᵢ sequence is a same nucleic acid sequence as the ZipAᵢ sequence or the ZipBᵢ sequence. In some embodiments, the SeqAᵢ sequence, the SeqBᵢ sequence and the SeqCᵢ sequence are specifically linked without any intervening sequences. In some embodiments, concatenation of the SeqAᵢ sequence, the SeqBᵢ sequence and the SeqCᵢ sequence without any intervening sequence is a functional genetic element.

In another aspect, the present disclosure provides a composition comprising a mixture described herein. In some cases, the composition comprises the first mixture, the second mixture, or the third mixture described herein.

In another aspect, the present disclosure provides a composition for synthesizing a plurality of n different polynucleotides, comprising:
a first mixture of at least n polynucleotides, wherein an ith polynucleotide comprises a SeqAᵢ sequence and a ZipAi sequence (where i=1 to n), and wherein the ZipAᵢ sequence is different from a ZipAⱼ sequence of a jth polynucleotide when i≠j; and
a second mixture of at least n polynucleotides, wherein an ith polynucleotide comprises a SeqBi sequence and ZipBi sequence (where i=1 to n), and wherein the ZipBᵢ sequence is different from a ZipBᵢ sequence of a jth polynucleotide when i≠j;
wherein, for each of i,
concatenation of the SeqAi sequence and the SeqBi sequence without intervening sequence is a functional genetic element, and
the ZipAᵢ sequence and the ZipBᵢ sequence are connector sequences for linking the SeqAᵢ sequence and the SeqBᵢ sequence.

In some embodiments, the first mixture and the second mixture are within a same compartment or a same mixture.

In some embodiments, the ZipAᵢ sequence and the ZipBᵢ sequence are specifically linked. In some embodiments, the ZipAᵢ sequence and the ZipBᵢ sequence are hybridized. In some embodiments, the ZipAᵢ sequence and the ZipBᵢ sequence are a same nucleic acid sequence, complementary nucleic acid sequences, or different nucleic acid sequences.

In some embodiments, the composition further comprises a third mixture of at least n polynucleotides, where an ith polynucleotide comprises a SeqCᵢ sequence and a ZipCᵢ sequence, and wherein the ZipCᵢ sequence is different from a ZipCᵢ sequence of a jth polynucleotide when i≠j.

In some embodiments, for each of i, concatenation of the SeqAᵢ sequence, the SeqBᵢ sequence, and the SeqCᵢ sequence without any intervening sequence is a functional genetic element.

In some embodiments, the ZipCᵢ sequence is a connector sequence for linking the SeqCᵢ sequence and a sequence comprising the SeqAᵢ sequence and the SeqBᵢ sequence. In some embodiments, the ZipCᵢ sequence is a same nucleic acid sequence as the ZipAᵢ sequence or the ZipBᵢ sequence. In some embodiments, the ZipCⱼ sequence is a different nucleic acid sequence from the ZipAᵢ sequence or the ZipBᵢ sequence. In some embodiments, the ZipCᵢ sequence, the ZipAᵢ sequence and the ZipBᵢ sequence are a same nucleic acid sequence.

In some embodiments, the first mixture, the second mixture and the third mixture are within a same compartment or a same mixture.

In some embodiments, the functional genetic element comprises a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, and/or a primer-extension gRNA for prime editing.

In another aspect, the present disclosure provides a composition comprising a polynucleotide having at least two double-stranded regions separated by a single-stranded region, wherein the at least two double-stranded regions comprise a first double-stranded region and a second double-stranded region, wherein the single-stranded region hybridizes with a stable strand such that the single-stranded region forms a loop stabilized by the stable strand to bring a 3′ end of the first double-stranded region and a 5′ end of the second double-stranded region to close proximity, and wherein the first double-stranded region and the second double-stranded region are from a same functional genetic element.

In some embodiments, the functional genetic element is a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, or a primer-extension gRNA for prime editing. In some embodiments, the 3′ end of the first double-stranded region and the 5′ end of the second double-stranded region are in close proximity for ligation. In some embodiments, the 3′ end of the first double-stranded region and the 5′ end of the second double-stranded region are joined. In some embodiments, the 3′ end of the first double-stranded region and the 5′ end of the second double-stranded region are ligated. In some embodiments, the single-stranded region comprises, from 5′ to 3′, a first segment and a second segment, and the stable strand comprises, from 5′ to 3′, a third segment and a fourth segment, and wherein the first segment hybridizes with the third segment and the second segment hybridizes with the fourth segment. In some embodiments, the method further comprises a plurality of polynucleotides, each having at least two double-stranded regions separated by a single-stranded region, wherein the at least two double-stranded regions comprise a first double-stranded region and a second double-stranded region, wherein the single-stranded region hybridizes with a stable strand such that the single-stranded region forms a loop stabilized by the stable strand to bring a 3′ end of the first double-stranded region and a 5′ end of the second double-stranded region to close proximity, and wherein the first double-stranded region and the second double-stranded region are from a same functional genetic element. In some embodiments, each polynucleotide of the plurality is a different functional genetic element. In some embodiments, the polynucleotide comprises three double-stranded regions separated by two single-stranded regions, each single-stranded region hybridizing with a stable strand. In some embodiments, the three double-stranded regions are from a same functional genetic element.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “Fig.”, and “FIGURE” herein) of which:

FIG. 1A and FIG. 1B depict an example method of synthesizing a plurality of polynucleotides of interest from three mixtures.

FIGS. 2A-2C depict an example method of synthesizing a plurality of polynucleotides of interest from one mixture containing three subpopulations of nucleic acid fragments.

FIG. 3A and FIG. 3B depict an example method of synthesizing pools of paired CDR3-J polynucleotides using the Zip-based methods described herein.

FIG. 4A and FIG. 4B depict an example method of synthesizing pools of T-cell receptor (TCR) genes using the Zip-based methods described herein.

FIG. 5 depicts a schematic workflow of the Zip-based methods for nucleic acid sequence assembly described herein.

FIG. 6A and FIG. 6B depict accuracy of Zip-based assembly. FIG. 6A depicts C3Ja1-C3Ja2 Zip ligation accuracy. FIG. 6B depicts C3Jb1-C3Jb2 Zip ligation accuracy. Each dot represents a species in the 583-plex Zip-guided assembly reaction. The horizontal and vertical values represent the relative concentration and assembly accuracy of each species. Small artificial noise was added to the horizontal and vertical value to resolve overlapping dots.

FIG. 7 depicts accuracy of successive Zip-based assembly. Each dot represents a species in the 583-plex Zip-guided assembly reaction. The horizontal and vertical values represent the relative concentration and cumulative accuracy of each species after two rounds of Zip-based assembly reactions. Small artificial noise was added to the horizontal and vertical value to resolve overlapping dots.

FIG. 8A and FIG. 8B depict characterization of successive Zip-based assembly product 414. FIG. 8A depicts accuracy of successive Zip-based assembly. Each dot represents a species in the pool 414. The horizontal and vertical values represent the relative concentration and cumulative accuracy of each species after two rounds of Zip-based assembly reactions, followed by removal of the intervening Zip sequences (Zipᵢ of FIG. 4A and FIG. 4B) between two Product Constituents ([ConAᵢ|CDR3Jαᵢ} and [ConBᵢ|CDR3Jβᵢ}). FIG. 8B depicts relative concentration of each species in family 414 versus the relative concentration of the corresponding species in family 319. Small artificial noise was added to the horizontal and vertical value to resolve overlapping dots.

FIGS. 9A-9H depict a scheme for 3-level successive Zip-based assemblies to form genes from 8 families of Zip-linked Product Constituents. Thick line represents one strand of DNA. Circle and triangle connected to the thick line represent 5′ and 3′ of the DNA, respectively. Dashed line indicates covalent bond linking two ends of a dsDNA molecule to form a circular dsDNA.

FIGS. 10A-10D depict gel images showing quality of assembly intermediates and products for the 3-level successive Zip-based assemblies to form ˜1,000 genes from 8 families of Zip-linked Product Constituents. FIG. 10A depicts gel image of intermediates and products formed during the level-1 assembly in Example 4. FIG. 10B depicts gel image of intermediates and products formed during the level-2 assembly in Example 4. FIG. 10C depicts gel image of intermediates and products formed during the level-2 assembly in Example 4. FIG. 10D depicts gel image of products after level-3 assembly.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, or within 2-fold, of a value. Where particular values are described in the application and claims, unless otherwise stated the term “about” meaning within an acceptable error range for the particular value should be assumed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “polynucleotide”, “nucleic acid” and “oligonucleotide” are used interchangeably in the present disclosure. They can refer to a polymeric form of nucleotides of various length. They may comprise deoxyribonucleotides and/or ribonucleotides, or analogs thereof. A polynucleotide may include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. A polynucleotide may have any three-dimensional structure and may perform various functions. A polynucleotide can have various configurations, such as linear, circular, stem-loop, and branched. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), circular RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Polynucleotides may include one or more nucleotide variants, including nonstandard nucleotide(s), non-natural nucleotide(s), nucleotide analog(s) and/or modified nucleotides.

The term “sequence,” as used herein, refers to the order of nucleotides in a nucleic acid molecule, or the order of amino acid residues of a peptide. A nucleic acid sequence can be a deoxyribonucleic acid (DNA) sequence or ribonucleic acid (RNA) sequence; can be linear, circular or branched; and can be either single-stranded or double-stranded. A sequence can be mutated such that it is different from a reference sequence (e.g., wildtype sequence). A sequence can be of any length, for example, between 2 and 1,000,000 or more amino acids or nucleotides in length (or any integer value there between or there above), e.g., between about 100 and about 10,000 nucleotides or between about 200 and about 500 amino acids or nucleotides. Any given nucleic acid sequence can encompass the sequence information of the given nucleic acid sequence and a reverse complement sequence of the given nucleic acid sequence. In some cases, a DNA sequence can encompass the sequence information of the corresponding RNA sequence that is transcribed from the DNA. The sequence can be alphabetical representation of a polynucleotide or polypeptide molecule. The sequence can be a piece of information that can be used by a computer processor. In some cases, the nucleic acid sequence may be used to refer to the physical nucleic acid molecule itself.

The term “blunt end,” as used herein, refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion having at least one nucleotide in length, referred to herein as an “overhang” or “sticky end.”

The terms “link” or “connect” are used interchangeably in the present disclosure. They refer to physically linking two or more nucleic acid molecules. The two or more nucleic acid molecules may be linked such that the two or more nucleic acid molecules form a continuous nucleic acid molecule. The two or more nucleic acid molecules can be covalently linked or non-covalently linked. Linking may be accomplished in a variety of manners, including formation of hydrogen bonds, ionic and covalent bonds, or van der Wals forces.

Percent (%) sequence identity with respect to a reference nucleic acid sequence (or peptide sequence) is the percentage of nucleotides (or amino acid residues in case of peptide sequence) in a candidate sequence that are identical with the nucleotides (or amino acid residues) in the reference nucleic acid sequence (or peptide sequence), after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTALW, ALIGN or Megalign (DNASTAR) software. Those skilled in the art can determine appropriate parameters for aligning sequences, including any algorithms needed to achieve maximal alignment over the full length of the sequences being compared.

The term “substantially the same” and its grammatical equivalents as applied to nucleic acid or amino acid sequences mean that a nucleic acid or amino acid sequence comprises a sequence that has at least 90% sequence identity or more, at least 95%, at least 98% or at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)).

Domain-level description of sequence: in the present disclosure, the polynucleotide sequence may be described at domain level. Each domain name can correspond to a specific polynucleotide sequence. For example, domain ‘A’ may have a sequence of 5′-TATTCCC-3′, domain ‘B’ may have a sequence of 5′-AGGGAC-3′, and domain ‘C’ may have a sequence of 5′-GGGAAGA-3′. In this case the polynucleotide having a sequence that is the concatenation of domains A, B, and C, can be written as [A|B|C}. The symbol ‘[’ denotes the 5′ end, the symbol ‘}’ denotes the 3′ end, and the symbol ‘|’ separates domain names. An ssDNA or a section of ssDNA having sequence ‘X’ can be referred to as [X}. An asterisk sign shows sequence complementarity. For example, domain [X*} is the reverse complement of domain [X}. The notation ds[X} can be used to describe a double-stranded DNA formed by [X} and [X*}. In some cases, especially in situation where it is not necessary to distinguish dsDNA and ssDNA, a dsDNA whose one strand has the sequence [X} may also be loosely referred to as [X}. A single-stranded RNA molecule or segment with the sequence identical to [X} (except replacing T with U) may also be referred to as [X}. Depending on the context, the domain name may refer to an exact sequence or describe a general function of a DNA or domain. For example, [RBS} may be used to describe a ribosome binding site, although the exact sequence for [RBS} may vary. Parentheses can be used to group a concatenation of domains, and the reverse-complement operation (denoted by ‘*’) can be applied to the concatenation by adding the ‘*’ following the closing parenthesis. For example, [(X|Y) *} is the same as [Y*|X*}. A double-stranded DNA formed by two strands [X} and [X*} can be written as [X}:[X*}. A double-stranded segment of a double-stranded DNA can be written in similar manner. For example, a dsDNA formed by [X|Y} and [Y*|X*} can be said to have double-stranded segments [X}:[X*} and [Y}: [Y*}. A double-stranded segment [X}:[X*} can also be called “double-stranded DNA [X}” or “dsDNA [X}” without creating ambiguity.

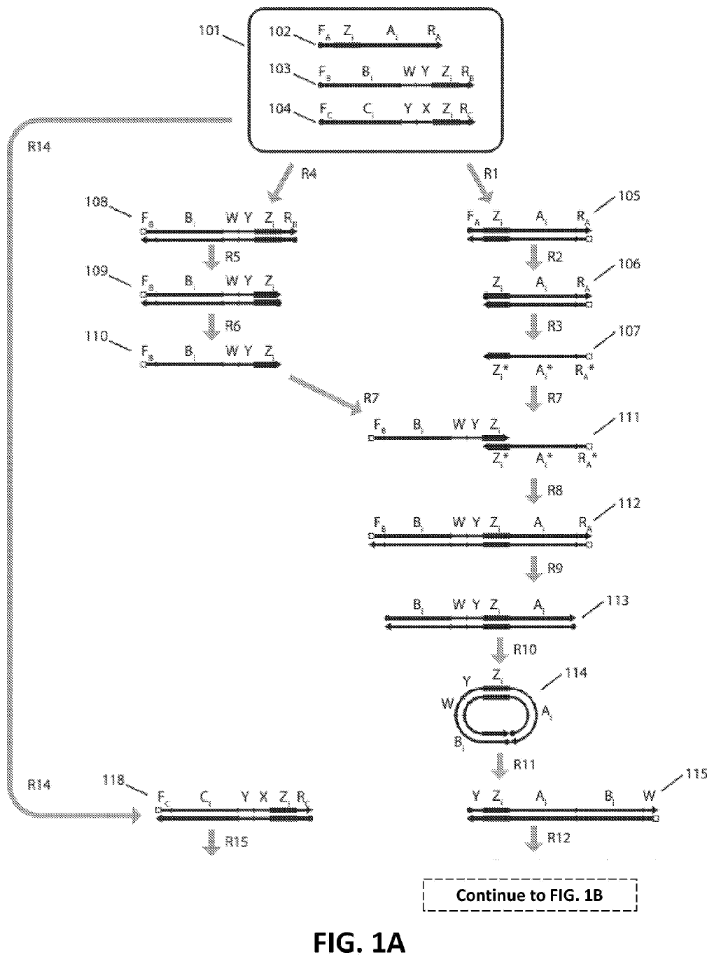

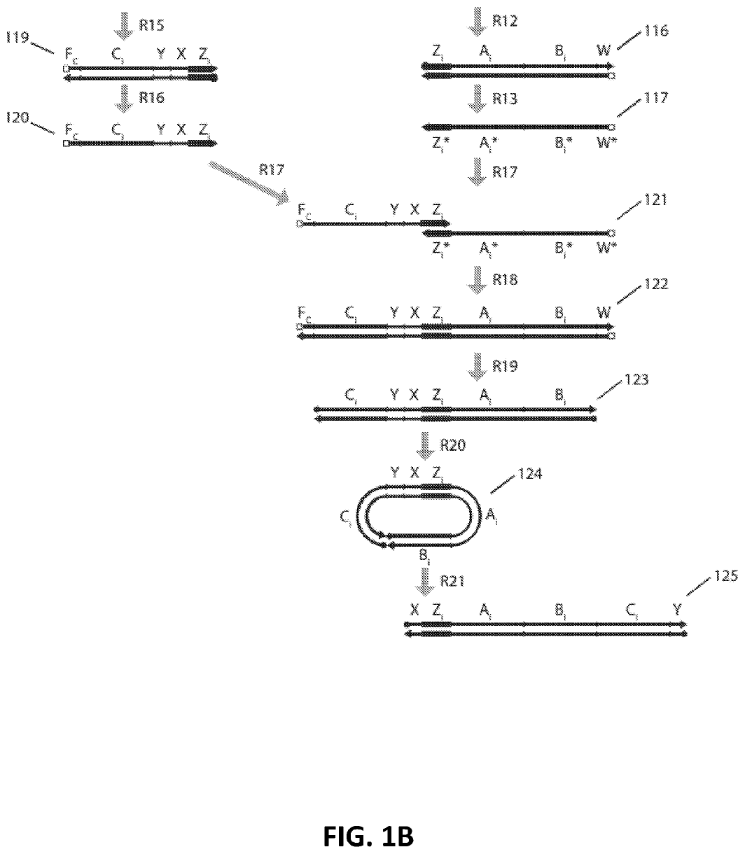

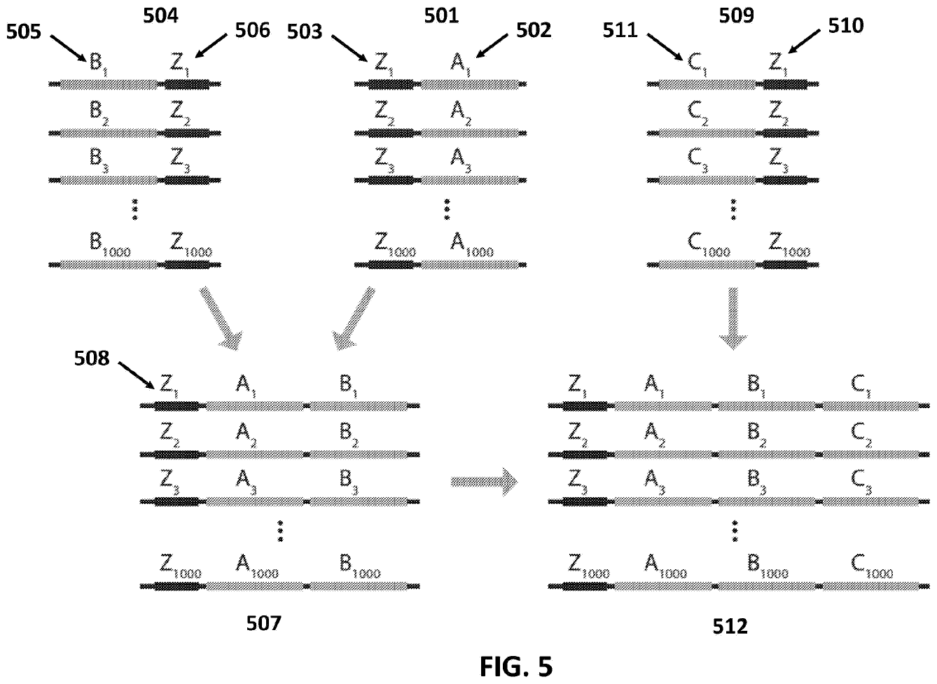

The term “a family of polynucleotides” or “a family of oligos,” as used herein, refers to a collection of polynucleotides that can be treated identically (e.g., subject to the same condition or procedure) in a reaction. A family of polynucleotides can have the same domain organization and only differ in Product Constituents and Zips. For example, in FIG. 1 and Example 1, all the polynucleotides in the pool (or mixture) 102 have the same domain organization of [F_{A}|Zᵢ|Aᵢ|R_{A}}, where F_{A} and R_{A} are identical in all polynucleotides, while Zᵢ and Aᵢ may have different sequences for different genes. All polynucleotides in 102 can be PCR-amplified using [F_{A}} and [R_{A}*} (e.g., identically). Therefore, the pool 102 can be called a family of polynucleotides. In fact, other than 101, each numbered pool in FIG. 1 can represent a family of polynucleotides. For another example, the numbered pool 501, 504, 507 or 509 in FIG. 5 can represent a family of polynucleotides.

Product Constituents, Zips and Operators: the oligos (or nucleic acid fragments) used to assemble genes of interest can be designed to contain three types of sequences: Product Constituents, Zips, and Operators. The term “Product Constituent,” as used herein, refers to a sequence that eventually become part of the final product. For example, in the process shown in FIG. 1 and Example 1, the final n products each have the sequence [Aᵢ|Bᵢ|Cᵢ} (where i=1 to n, n being the number of genes assembled simultaneously). The sequences Aᵢ can be contributed by the family of oligos 102 within the oligo pool 101. Since Aᵢ is part of the final product [Aᵢ|Bᵢ|Cᵢ}, the Aᵢ domain can be considered a Product Constituent (also called Product Constituent domain, Product Constituent sequence, or Product Constituent segment) in the ith species within the family 102. Similarly, Bᵢ domains in 103 and Cᵢ domains in 104 can also be Product Constituents.

The term “Zip,” “Zip domain,” or “Zip sequence” refers to a domain used to guide gene-specific assembly of two or more polynucleotides whose sequence can be arbitrarily designed. Zips can be connector sequences. The term “gene-specific,” as used herein, refers to the fact that when multiple genes (e.g., polynucleotides of interest) are assembled in the same homogenous assembly reaction, the assembly of two or more polynucleotides contributing to the same gene (in the correct order and orientation) is wanted, while assembly of two or more polynucleotides contributing to different genes (regardless of whether the order or orientation is correct) is unwanted. Because the Zip-guided assembly can be gene-specific, Zips used to assemble polynucleotides for different genes may be different. For example, in step R7 of FIG. 1, Z₁, which is used to assemble gene [A₁|B₁|C₁} (e.g., to assemble the A₁-containing polynucleotide within the family 107 and the B₁-containing polynucleotide within the family 110) is different from Z₂, which is used to assemble gene [A₂|B₂|C₂} (e.g., to assemble the A₂-containing polynucleotide within the family 107 and the B₂-containing polynucleotide within the family 110). More generally, if n genes are assembled in homogenous assembly reactions as described herein, for any i and j (where 1≤i≤n and 1≤j≤n), if i≠j, then the ith Zip (e.g., Zipᵢ or Zᵢ) and the jth Zip (e.g., Zipⱼ or Zⱼ) have different sequences. In the case of the example shown in FIG. 1, if i≠j, then Zᵢ and Zⱼ have different sequences. In some cases, after assembling two families of polynucleotides in a reaction, Zips within each family can be re-used to assembly additional polynucleotides in subsequent reactions. The length of the Zips can vary. In some cases, Zips may have the length of 4 to 50 nt.

The term “Operator,” as used herein, refers to a domain used to process a family of polynucleotides in the same way. Operators can have sequences that are common to all polynucleotides in the same family. For example, the domains F_{A} and R_{A} in the family 102 (having the sequence of [F_{A}|Zᵢ|Aᵢ|R_{A}}) can be Operators. Operators may serve different roles. A common role of Operators may be the primer binding site. For example, the domains F_{A} and R_{A} in the family 102 can serve as primer binding sites to amplify all polynucleotides of the family 102. Operators may also contain restriction sites (e.g., Operators ADS_{L} and ADSR, see Example 2 for details. Operators can also be arbitrarily designed using the same process of Zip design, except that the sequence constraints (e.g., a restriction site may be present at a defined position, or the last base of a domain can be dT) need to be considered and implemented during the generation of the initial random sequences.

The letter “n” (italicized or non-italicized), used in the context of a plurality of at least n polynucleotides, donates the total number of polynucleotides of interest to be assembled or synthesized using the methods provided herein. In various embodiments, n is an integer equal to or greater than 2. For example, a plurality of at least n polynucleotides can be two or more polynucleotides. If 1000 polynucleotides of interest are synthesized, then n=1000. As used herein, a given polynucleotide of the plurality being synthesized or a given polynucleotide of a mixture (e.g., a pool or a family of polynucleotides) during the synthesis can be referred to as an ith polynucleotide. For example, the ith polynucleotide can be a first polynucleotide (when i=1), a second polynucleotide (when i=2), a third polynucleotide (when i=3) . . . or a nth polynucleotide (when i=n). Sequences or subsequences (e.g., Constituents, Zips or Operators) used to assemble or synthesize the ith polynucleotide can be denoted with “i” (in various cases, as a subscript) following the name of the sequences or subsequences. For example, Zip sequence of the ith polynucleotide can be denoted as Zipᵢ or Zᵢ. In some cases, another given polynucleotide of the plurality being synthesized or a given polynucleotide of a mixture (e.g., a pool or a family of polynucleotides) during the synthesis can be referred to as a jth polynucleotide. The jth polynucleotide denotes a different polynucleotide from the ith polynucleotide. The Zip sequences of the jth polynucleotide can be different from the Zip sequence of the ith polynucleotide. For example, a mixture can comprise a first polynucleotide comprising Zip₁ sequence and a second polynucleotide comprising Zip₂ sequence, where Zip₁ sequence is different form Zip₂ sequence. In other words, if i≠j, then the ith Zip (e.g., Zipᵢ or Zᵢ) and the jth Zip (e.g., Zipⱼ or Zⱼ) have different sequences. For any given polynucleotide within a mixture (e.g., a family of polynucleotides), the Zip sequence can be unique and can be different from any other Zip sequence of any other polynucleotide. As used herein, “i” and “j” can be any integer from 1 to n (the total number of polynucleotides of interest to be synthesized). For example, “i” or “j” can be 1, 2, 3, 4, 5, 6, 7, 8, 9 . . . or n.

The term “Assembly” or “assembly process,” as used herein, refers to a reaction or a series of reactions in which the Product Constituents of two or more polynucleotide molecules are linked to form a continuous (e.g., copiable by a DNA or RNA polymerase) and longer Product Constituent. Each of the individual reaction used to complete an assembly process can be called an assembly reaction. An assembly process may include a ligation reaction or a primer extension reaction. For example, in assembly reactions R7 through R10 of FIG. 1, the following events can occur: First, each polynucleotide of the family 110 can be hybridized with its corresponding polynucleotide in the family 107. Then, the two hybridized polynucleotides can then use each other as template to undergo primer extension reaction to form family 112. Next, through adaptor removal reaction and circularization reaction, Aj and Bᵢ (the two Product Constituents for each gene, initially carried by 107 and 110, respectively) can be ligated to form the longer Product Constituents [Ai|Bi} in the family 114.

### Overview

Gene synthesis is a broadly enabling technology for life science research and health care. Despite of decades of improvement, currently gene synthesis cost can be prohibitive for many applications where thousands of genes need to be synthesized. As a non-limiting example, to find a better-performing version of an industrial enzyme, one may contemplate testing 10,000 naturally existing candidate enzymes whose are homologous to the original enzyme. The coding sequences for the 10,000 candidate enzymes may be found by searching a gene sequence database. However, to test the function of these enzymes, their genes may be synthesized first. In 2021, the typical cost of gene synthesis can be about $0.09 per base pair (bp). Suppose the average length of the candidate enzyme is 3,000 bp (or 3 kb), a total of 30,000,000 bp of genes may be synthesized, costing $2.7 million. Such cost may be prohibitive in many situations or applications.

One breakthrough in the area of gene synthesis over the past decade or so includes high throughput short oligonucleotide (oligo) pool synthesis, where tens of thousands (or more) of short—in some cases, 50 to 300 nucleotide (nt)—oligos can be synthesized on a microarray, cleaved from the microarray and delivered as a pool (or mixture) of oligos. However, to assemble these oligo pools into thousands of long genes in a controlled, high-throughput, and high-quality manner is still an unsolved challenge. The present disclosure can address this challenge by utilizing a large number (in many cases, hundreds, thousands or more) of designed connector domains or sequences, also referred to as Zips in the present disclosure.

In the existing methods, oligos needed to assemble only one gene are used in one assembly reaction or those oligos are mixed in one compartment. For example, if oligos named A₁, B₁, C₁ and D₁ are used to assemble gene 1, and oligos A₂, B₂, C₂ and D₂ are used to assemble gene 2, one can typically mix oligos A₁, B₁, C₁ and D₁ in one reaction (e.g., overlapping PCR) and mix oligos A₂, B₂, C₂ and D₂ in a separate reaction. In other words, in the above situations, oligos A₂, B₂, C₂ and D₂ are mixed in a different compartment separate from the reaction containing oligos A₁, B₁, C₁ and D₁. If all 8 oligos are mixed in one reaction (or one compartment), one oligo belonging to gene 1 may be inadvertently assembled with an oligo belonging to gene 2, leading to an erroneous product. This error can be called cross-gene misassembly. In this manner, if n genes need to be assembled, at least n assembly reactions (which are in separate compartments) need to be set up. This can be tedious and costly when n is large (e.g., n>100). While methods such as DropSynth exists to generate a large number (e.g., millions) of compartments (e.g., droplets), each one of which undergoes a separate assembly reaction (e.g., overlapping PCR), the length, quality, concentration uniformity of the assembled genes may not be satisfactory for most applications. This is partly because the size the contents of the droplets can be hard to precisely control.

In the present disclosure, methods and compositions are provided to assemble n genes with much less than n (e.g., less than n/10, less than n/20, less than n/30, less than n/40, or less than n/50) assembly reactions. Here, each of the assembly reaction may be a homogeneous mixture where any two molecules in the mixture may make contact. In other words, each assembly reaction may happen in one compartment, which may not comprise additional compartments, although these methods in some cases may not preclude creating additional compartments. In the present disclosure, oligos contributing to different genes may be mixed in one homogenous assembly reaction, where cross-gene misassembly can be minimized or prevented by meticulous sequence and reaction design. In the example given above, all 8 oligos (A₁, B₁, C₁, D₁, A₂, B₂, C₂ and D₂) can be processed in certain way and then mixed in one homogeneous reaction to produce gene 1 and gene 2. In fact, oligos needed to assemble as many as 1,000 or more genes can be processed and mixed in one homogenous assembly reaction to make desired assembly products (e.g., FIG. 5).

In some cases, the overall strategy to reduce the number of assembly reactions can be to manipulate polynucleotides belonging to the same family together (in a series of homogenous reactions), rather than to manipulate polynucleotides belonging to the same gene. For example, if the ith gene requires oligos Ai, Bi, Ci and Di (i=1 to n), all the n polynucleotides Ai (i=1 to n) can be considered one family. Similarly, all the n polynucleotides Bi (i=1 to n) can be considered one family, so on and so forth. Therefore, only four families of polynucleotides may be of concern for assembling the ith gene that requires oligos Ai, Bi, Ci and Di (i=1 to n). A series of 5 to 20 reactions, each containing one or a few families of polynucleotides, may be needed to process each family or several families together. After this series of reactions, all of the n genes can be assembled.

In various embodiments, the polynucleotide assembly reactions provided in the present disclosure can be carried out in a liquid. The polynucleotide assembly reactions provided herein may not be performed on a solid support or a solid surface. The nucleic acid fragments used to assemble various polynucleotides of interest can be soluble in the assembly reactions and may not be fixed on a solid support or a solid surface.

### Methods for Synthesizing Polynucleotides

The present disclosure provides methods for synthesizing or assembling a plurality of different polynucleotides of interest from two or more fragments in a mixture (e.g., a same mixture or a single mixture), in the same compartment, or in a single compartment. The methods provided herein may not require microarray or chip for nucleic acid synthesis. The methods provided herein may not require separating fragments for assembling each gene into separate compartments (e.g., in emulsions). The plurality of polynucleotides of interest can be synthesized or assembled in bulk in one compartment. The plurality of polynucleotides of interest can be synthesized or assembled in solution. The plurality of polynucleotides of interest can be assembled or synthesized from various fragments in a same mixture without non-specific linkages or cross-gene misassembly. The plurality of polynucleotides can comprise different nucleic acid sequences. In some cases, each polynucleotide of the plurality synthesized or assembled comprise a unique sequence that is different from other sequences in the plurality.

For example, the methods provided herein can be used to synthesize a plurality of n polynucleotides or a plurality of at least n polynucleotides, where n can be an integer that is equal to or greater than 2. In some cases, n denotes the total number of polynucleotides of interest that are synthesized in a mixture. In some cases, the plurality of n different polynucleotides is synthesized in a single compartment or the same mixture. In some cases, the plurality of n polynucleotides synthesized can comprise at least 2, 5, 10, 20, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000 or more different sequences. As described herein, “Seq” can be used to denote the sequence of each polynucleotide of the plurality of n polynucleotides. The Seq sequence can be the sequence of interest or the sequence desired to be synthesized. And, an ith polynucleotide of the plurality can comprise a Seqᵢ sequence (where i=1 to n). For example, a first polynucleotide of the plurality can comprise a Seq₁ sequence, a second polynucleotide of the plurality can comprise a Seq₂ sequence, a third polynucleotide of the plurality can comprise a Seq₃ sequence . . . and an nth polynucleotide of the plurality can comprise a Seqₙ sequence. As used herein, “Seq” followed by a letter such as SeqA, SeqB,SeqC . . . or SeqZ can be used to denote nucleic acid fragments that are used to synthesize the polynucleotide of interest containing a Seq sequence. For simplicity, in some cases, a single letter without “Seq” may be used to denote the sequence of interest. For example, A₁, A₂, A₃, A1000, B₁, B₂, B₃, and B₁₀₀₀ in figures described herein can be used to denote the sequences of interest. For each i, the Seqᵢ sequence can be synthesized from two or more fragments including SeqAᵢ , SeqBᵢ, SeqCᵢ . . . and/or SeqZᵢ. For example, the Seqi sequence can be synthesized from a sequence containing SeqAᵢ and a sequence containing SeqBᵢ.

As an example shown in FIG. 5, the Seqᵢ sequence (e.g., the sequences in mixture 512 of FIG. 5) can be synthesized from a sequence containing SeqAᵢ , a sequence containing SeqBI, and a sequence containing SeqCᵢ . The synthesized Seqᵢ sequence can comprise a

SeqAᵢ sequence, a SeqBᵢ sequence and a SeqCᵢ sequence. In some cases, the synthesized Seqᵢ sequence can comprise a SeqAᵢ sequence, a SeqBᵢ sequence and a SeqCᵢ sequence sequentially from 5′ end to 3′ end.

The methods provided herein can comprise providing a first mixture of at least n polynucleotides 501, where an ith polynucleotide comprises a SeqAᵢ sequence 501 and a ZipAᵢ sequence 503. The first mixture of at least n polynucleotides can be a family of polynucleotides. The SeqAᵢ sequence can be a portion of the Seqᵢ sequence of interest, and the ZipAᵢ sequence can be a connector sequence used for linking the SeqAᵢ sequence with another sequence. Each connector sequence in the first mixture can comprise a unique sequence that is different from other connector sequences in the first mixture. For example, the ZipAᵢ sequence can be different from a ZipAⱼ sequence when i≠j. As used herein, i or j can be an integer from 1 to n (n can be the total number of polynucleotides to be synthesized), which can be used to denote any given polynucleotide of a mixture of polynucleotides. Next, a second mixture of at least n polynucleotides 504 can be provided. In the second mixture, an ith polynucleotide can comprise a SeqBᵢ sequence 505 and a ZipBᵢ sequence 506. The second mixture of at least n polynucleotides can be a family of polynucleotides. The SeqBᵢ sequence can be a portion of the Seqᵢ sequence of interest, and the ZipBᵢ sequence can be a connector sequence used for linking the SeqBᵢ sequence with another sequence. Each connector sequence in the first mixture can comprise a unique sequence that is different from other connector sequences in the second mixture. The ZipBᵢ sequence can be different from a ZipBᵢ sequence when i≠j. Next, the first mixture 501 and the second mixture 504 can be contacted, thereby generating a third mixture of n polynucleotides 507. In the third mixture, an ith polynucleotide comprises a SeqAᵢ sequence, a SeqBᵢ sequence and a ZipABᵢ sequence 508. The ZipABᵢ sequence can be different from a ZipABⱼ sequence when i≠j. The ZipABᵢ sequence may not be flanked by the SeqAᵢ sequence and the SeqBᵢ sequence. The ZipABᵢ sequence may be at a terminus of the ith polynucleotide comprising the SeqAᵢ sequence, the SeqBᵢ sequence and the ZipABᵢ sequence. Next, a fourth mixture of at least n polynucleotides 509 can be provided. The fourth mixture of at least n polynucleotides can be a family of polynucleotides. In the fourth mixture, an ith polynucleotide can comprise a SeqCᵢ sequence 511 and a ZipCᵢ sequence 510. The SeqCᵢ sequence can be a portion of the Seqᵢ sequence of interest, and the ZipCᵢ sequence can be a connector sequence used for linking the SeqCᵢ sequence with another sequence. Each connector sequence in the first mixture can comprise a unique sequence that is different from other connector sequences in the second mixture. The ZipCᵢ sequence can be different from a ZipCᵢ sequence when i≠j. Next, the third mixture 507 and the fourth mixture 509 can be contacted, thereby generating a fifth mixture of n polynucleotides 512, where an ith polynucleotide comprises the Seqᵢ sequence comprising the SeqAᵢ sequence, the SeqBᵢ sequence and the SeqCᵢ sequence. In some cases, a sixth mixture, a seventh mixture or more may be used to add more fragments onto already synthesized polynucleotides to generate the polynucleotides of interest.

In some cases, the methods can comprise providing a first mixture of at least two polynucleotides comprising a first polynucleotide and second polynucleotide. The first polynucleotide of the first mixture can comprise a SeqA₁ sequence and a ZipA₁ sequence and a second polynucleotide of the first mixture can comprise a SeqA₂ sequence and a ZipA₂ sequence. The ZipA sequence can be different from the ZipA₂ sequence. In some cases, a second mixture of at least two polynucleotides can be provided. The second mixture can comprise a first polynucleotide and a second polynucleotide. The first polynucleotide of the second mixture can comprise a SeqBᵢ sequence and a ZipB₁ sequence and the second polynucleotide of the second mixture can comprise a SeqB₂ sequence and a ZipB₂ sequence. The ZipB₁ sequence can be different from the ZipB₂ sequence. In some cases, a third mixture of at least two polynucleotides can be provided. The third mixture can comprise a first polynucleotide and a second polynucleotide. The first polynucleotide of the third mixture can comprise a SeqC₁ sequence and a ZipC₁ sequence and the second polynucleotide of the third mixture can comprise a SeqC₂ sequence and a ZipC₂ sequence. The ZipC₁ sequence can be different from the ZipC₂ sequence. In some cases, an additional one or more mixtures can be provided. The polynucleotides within each of the mixtures can be mixed to generate final product polynucleotides.

In some cases, the methods provided herein can comprise providing a first mixture of at least n polynucleotides. An ith polynucleotide of the first mixture can comprise a SeqAᵢ sequence and a ZipAᵢ sequence, and the ZipAᵢ sequence can be different from a ZipAⱼ (where j=1 to n) sequence of a jth polynucleotide when i≠j. Next, a second mixture of at least n polynucleotides can be provided. An ith polynucleotide of the second mixture can comprise a SeqBᵢ sequence and a ZipBᵢ sequence, and the ZipBᵢ sequence can be different from a ZipBᵢ sequence of a jth polynucleotide when i≠j. Next, the first mixture and the second mixture can be contacted to generate a third mixture of at least n polynucleotides, wherein an ith polynucleotide comprises a SeqAᵢ sequence, a SeqBᵢ sequence and a ZipABᵢ sequence, and wherein the ZipABᵢ sequence is different from a ZipABᵢ sequence of a jth polynucleotide when i≠j. Next, a fourth mixture of at least n polynucleotides can be provided, wherein an ith polynucleotide comprises a SeqCᵢ sequence and a ZipCᵢ sequence, and wherein the ZipCᵢ sequence is different from a ZipCᵢ sequence of jth polynucleotide when i≠j. Next, the third mixture and the fourth mixture can be contacted to generate a fifth mixture of at least n polynucleotides, wherein an ith polynucleotide comprises the Seqᵢ sequence comprising the SeqAᵢ sequence, the SeqBᵢ sequence and the SeqCᵢ sequence. In some cases, generating the third mixture of at least n polynucleotides comprises specifically linking, for each i, the ZipAᵢ sequence and the ZipBᵢ sequence. In some cases, generating the fifth mixture of at least n polynucleotides comprises specifically linking, for each i, the ZipCᵢ sequence and the ZipABᵢ sequence.

The SeqAᵢ sequence, the SeqBᵢ sequence and the SeqCᵢ sequence of the Seqᵢ sequence can be linked seamlessly. As used herein, “seamless” used in the context of gene fusion or gene assembly refers to processes that allow two or more nucleic acid fragments to be joined precisely so that no unwanted (or intervening) nucleotides are added at the junctions between the nucleic acid fragments. For example, the Seqᵢ sequence can comprise the SeqAᵢ sequence and the SeqBᵢ sequence without an intervening sequence (e.g., a Zip sequence) in between the SeqAᵢ sequence and the SeqBᵢ sequence. The Seqᵢ sequence can comprise the SeqBᵢ sequence and the SeqCᵢ sequence without an intervening sequence in between the SeqBᵢ sequence and the SeqCᵢ sequence. In some cases, the Seqᵢ sequence with an intervening sequence in between the SeqAᵢ sequence and the SeqBᵢ sequence or the SeqBᵢ sequence and the SeqCᵢ sequence is not a functional genetic element. The Seqᵢ sequence can comprise the SeqAᵢ sequence, the SeqBᵢ sequence and the SeqCᵢ sequence sequentially from the 5′ end to the 3′ end.

For each i, the SeqAᵢ sequence, the SeqBᵢ sequence and the SeqCᵢ sequence of the Seq, sequence can be linked specifically. The Zip sequences used in the methods can be connector sequences for linking one fragment with another fragment in a mixture specifically. As used herein, “Zip” followed by a letter such as ZipA, ZipB, ZipC . . . or ZipZ can be used to denote the connector sequence of a sequence of interest (e.g., a nucleic acid fragment containing corresponding Seq sequence). For simplicity, in some cases, a single letter “Z” may be used to denote the connector sequence. For example, Z₁, Z₂, Z₃, and Z₁₀₀₀ in figures described herein can be used to denote the connector sequences. For example, in some cases, for each i, the ZipAᵢ sequence and the ZipBᵢ sequence can be connector sequences for linking the SeqAᵢ sequence and the SeqBᵢ sequence. Each Zip sequence can be used to specifically link one fragment (e.g., SeqA) to another fragment (e.g., SeqB) such that the synthesized sequence containing SeqA and SeqB is a functional genetic element. For example, the Zip sequence can be used to specifically link a fragment containing SeqA₁ to another fragment containing SeqB1 such that the synthesized sequence containing SeqA₁ and SeqB1 is a functional genetic element. SeqA₁ and SeqBᵢ can be from the same polynucleotide of interest to be synthesized or the same functional genetic element to be synthesized. The Zip sequence can be used to prevent or minimize misassembly of the fragments. For example, the Zip sequence may not link a fragment containing SeqA₁ to another fragment containing SeqB2. The Zip sequences used in a mixture can be re-used in another mixture. For each i, the ZipCᵢ sequence and the ZipABᵢ sequence can be connector sequences for linking the SeqCᵢ sequence and a sequence comprising the SeqAᵢ sequence and the SeqBᵢ sequence. For each i, the ZipAᵢ sequence and the ZipBᵢ sequence can be a same nucleic acid sequence. In some cases, the ZipAᵢ sequence and the ZipBᵢ sequence may be substantially the same. For each i, the ZipAᵢ sequence and the ZipBᵢ sequence can be complementary (e.g., fully or partially complementary) or the ZipBᵢ sequence is a reverse complement of the ZipAᵢ sequence. For each i, the ZipAᵢ sequence and the ZipBᵢ sequence can be different nucleic acid sequences. For each i, the ZipABᵢ sequence can be a same nucleic acid sequence as the ZipAᵢ sequence or the ZipBᵢ sequence. For each i, the ZipABᵢ sequence, the ZipAᵢ sequence, and the ZipBᵢ sequence can be a same nucleic acid sequence (e.g., Zip sequences in FIG. 5). In some cases, the ZipABᵢ sequence, the ZipAᵢ sequence, and the ZipBᵢ sequence may be substantially the same. For each i, the ZipABᵢ sequence can be a different nucleic acid sequence from the ZipAᵢ sequence or the ZipBᵢ sequence. The ZipABᵢ sequence, the ZipAᵢ sequence, and the ZipBᵢ sequence can be different nucleic acid sequences. The ZipCᵢ sequence and the ZipABᵢ sequence can be a same nucleic acid sequence. The ZipCᵢ sequence and the ZipABᵢ sequence may be substantially the same. The ZipCᵢ sequence and the ZipABᵢ sequence can be complementary. The ZipCᵢ sequence and the ZipABᵢ sequence can be different nucleic acid sequences.

The connector sequence or Zip sequence described herein can be of various length. For example, the connector sequence or Zip sequence can be at least about 2, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 250, 280, 300, 350, 400, 450 or more nucleotides in length. The connector sequence or Zip sequence can be at most about 500, 450, 400, 350, 300, 250, 200, 150, 100, 50, 20 or less nucleotides in length. The connector sequence or Zip sequence can be from 2 to 50, from 10 to 60, from 5 to 100, from 10 to 200, from 2 to 100, from 5 to 200, from 5 to 300, or from 5 to 400 nucleotides in length. For example, in some cases, for each i, the ZipAᵢ sequence, the ZipBᵢ sequence, the ZipABᵢ sequence, or the ZipCᵢ sequence can be from 5 nucleotides to 200 nucleotides in length.

The nucleic acid fragments (e.g., the SeqAᵢ sequence, the SeqBᵢ sequence, or the SeqCᵢ sequence, etc.) used to synthesize the polynucleotide of interest can be of various length. For example, the nucleic acid fragments can be at least about 5, 10, 15, 20, 25, 30, 35, 40, 45, 50, 55, 60, 65, 70, 75, 80, 85, 90, 95, 100, 150, 200, 300, 400, 500, 600, 700, 800, 900, 1,000, 2,000, 3,000, 4,000, 5,000, 6,000, 7,000, 8,000, 9,000, 10,000 or more nucleotides in length. The nucleic acid fragments can be from 5 to 50, from 5 to 100, from 5 to 200, from 5 to 500, from 5 to 1,000, from 5 to 2,000, from 5 to 5,000, from 5 to 10,000, from 5 to 50,000, from 10 to 200, from 10 to 500, from 10 to 1,000, from 10 to 5,000, from 10 to 10,000, from 100 to 1,000, from 200 to 5,000, or from 200 to 10,000 nucleotides in length. For example, in some cases, for each i, the SeqAᵢ sequence, the SeqBᵢ sequence, or the SeqCᵢ sequence is from 5 nucleotides to 5,000 nucleotides in length.

As described herein, the first mixture and the second mixture can be contacted, thereby generating a third mixture of n polynucleotides. Various methods, including hybridization, primer extension and ligation, can be used to generate the third mixture of n polynucleotides from the first mixture and the second mixture. In some cases, generating the third mixture of n polynucleotides comprises linking (e.g., specifically linking), for each i, the ZipAᵢ sequence and the ZipBᵢ sequence. The linking can be specific such that ZipAᵢ sequence links to ZipBᵢ sequence but does not link to ZipBᵢ sequence when i≠j. In some cases, the ZipAᵢ sequence and the ZipBᵢ sequence are the same or complementary. In such cases, linking can comprise hybridizing, for each i, the ZipAᵢ sequence and the ZipBᵢ sequence (e.g., 111 of FIG. 1A). In some cases, the ZipAᵢ sequence and the ZipBᵢ sequence are different. In such cases, a bridging stand which can hybridize with both the ZipAᵢ sequence and the ZipBᵢ sequence can be used to link the ZipAᵢ sequence and the ZipB sequence. Next, a free 3′ end of the ZipAᵢ sequence or the ZipBᵢ sequence can be extended using the ith polynucleotide from the first mixture or the second mixture as a template to generate the ith polynucleotide comprising the SeqAᵢ sequence, the SeqBᵢ sequence and the ZipABᵢ sequence (e.g., 112 of FIG. 1A). Next, the ith polynucleotide comprising the SeqAᵢ sequence, the SeqBᵢ sequence and the ZipABᵢ sequence can be circularized to generate a circularized polynucleotide (e.g., 114 of FIG. 1A). In some cases, circularizing the ith polynucleotide comprising the SeqAᵢ sequence, the SeqBᵢ sequence and the ZipABᵢ sequence comprises circularizing the ith polynucleotide by a ligase. In some cases, a blunt-end DNA ligase can be used. Examples of blunt-end DNA ligase include, but are not limited to, T4 DNA ligase, T3 DNA ligase and Taq DNA ligase. In some cases, a single-stranded DNA (ssDNA) ligase may be used. Examples of ssDNA ligase include, but are not limited to, CircLigase or CircLigase II. Next, the circularized polynucleotide can be linearized. In some cases, linearizing the circularized product comprises cutting the circularized polynucleotide (e.g., by an enzyme) or amplifying the circularized polynucleotide using polymerase chain reaction (PCR) such as inside-out PCR (e.g., 115 of FIG. 1A). An inside-out PCR refers to a PCR using a circular DNA as template and a primer pair that generate a PCR product whose length is more than half of the length of the circular DNA. For example, in R11 of FIG. 1 and Example 1, amplification of circular DNA family 114, using [Y} and [W*} as primers can generate 115, whose length is the same as (e.g., more than half of) 114. Thus, the PCR reaction R11 can be called an inside-out PCR. In some cases, the circularized product can be linearized such that the ZipABᵢ sequence is not flanked by the SeqAᵢ sequence and the SeqBᵢ sequence. Next, the linearized product can be subject to adaptor removal reaction and followed by ssDNA generation reaction to generate the third mixture (e.g., 117 of FIG. 1A). For example, in some cases, the ZipABᵢ sequence can be exposed on a terminus of the ith polynucleotide comprising the SeqAᵢ sequence, the SeqBᵢ sequence and the ZipABᵢ sequence. Exposing the ZipABᵢ sequence can be done by cutting a terminal region adjacent to the ZipABᵢ sequence by an enzyme. FIG. 1A shows an example of contacting a first mixture 107 and a second mixture 110 to generate a third mixture 117.

As described herein, the third mixture 507 can be contacted with a fourth mixture 509 to generate a fifth mixture of n polynucleotides 512 (e.g., FIG. 5). In some cases, generating the fifth mixture of n polynucleotides comprises linking, for each i, the ZipCᵢ sequence and the ZipABᵢ sequence. In some cases, linking comprising hybridizing the ZipCᵢ sequence and the ZipABᵢ sequence. Similar operations as described above for the third mixture of n polynucleotides and the fourth mixture of n polynucleotides can be used. For example, as shown in FIG. 1B, a third mixture 117 can be contacted with a fourth mixture 120 to generate the fifth mixture 125 by performing hybridization, primer extension, adapter removal reaction, circularization and linearization. Similar operations can be repeated for a sixth mixture, a seventh mixture or more if additional fragments need to be added to the already synthesized polynucleotides. Optionally, the methods described herein can further comprising removing the ZipCᵢ sequence and the ZipABᵢ sequence, thereby generating the ith polynucleotide comprising the Seqᵢ sequence comprising the SeqAᵢ sequence, the SeqBᵢ sequence and the SeqCᵢ sequence. In some cases, removing the Zip sequences can comprise amplifying only the region containing the SeqAᵢ sequence, the SeqBᵢ sequence and the SeqCᵢ sequence. For example, the 5′ end of the SeqAᵢ sequence can comprise a common sequence and the 3′ end of the SeqCᵢ sequence can also comprise a common sequence different from the common sequence of the SeqAᵢ sequence. A pair of primers targeting the two common sequences can be used to amplify the sequences containing the SeqAᵢ sequence, the SeqBᵢ sequence and the SeqCᵢ sequence. For example, as shown in FIG. 1B, a pair of primers can be used to amplify the sequences containing only the SeqAᵢ sequence, the SeqBᵢ sequence and the SeqCᵢ sequence (without X, Zᵢ, and Y) in the mixture 125.

In many cases, an Operator domain at the 5′ end or 3′ end of a family of polynucleotides needs to be removed so that a Product Constituent or a Zip can be at the 5′ end or 3′ end. These reactions can be called “adaptor removal reactions” or “Operator removal reactions,” which can ensure the seamless ligation of Product Constituents or can improve the specificity of Zip-based assembly. For example, in R5 of FIG. 1 and Example 1, the Operator domains [R_{B}} and [R_{B}*} (on the top and bottom strands, respectively) of 108 can be removed to form 109. As a result, the Zip domain Zᵢ on the top strands of 109 can be at the 3′ end and can eventually extend (e.g., R8) without the hinderance of [R_{B}}. In another adaptor removal reaction, Operators [F_{B}}:[F_{B}*} and [R_{A}}:[R_{A}*} can be removed from 112 to form 113, to ensure that Aᵢ and Bᵢ can be seamlessly ligated to form [Aᵢ|Bᵢ} in 114. Several methods can be used to remove an Operator domain from a polynucleotide. For example, a Type IIS restriction site can be placed near the end of an Operator so that digestion of the dsDNA containing the Operator by the corresponding restriction enzyme can remove the Operator. For example, the last 8 bases of [F_{B}} and [R_{A}*} can have the following sequence: 5′-GAAGACNN-3′ where the underlined sequence is a recognition site of Type IIS restriction enzyme BbsI and N can be any base. In this case, treating dsDNA family 112 with BbsI may remove the Operator domain [F_{B}}:[F_{B}*} and [R_{A}}: [R_{A}*}, fulfilling the function of reaction R8. This process can create a 5′ overhang on both ends. The 5′ overhangs can be designed to have complementary sequence, originated from the final product, and facilitate the ensuing ligation reaction. In addition to BbsI, other Type IIS restriction enzymes can also be used, such as Bsal, BsmBI, BtgZI, and FokI. The full list of Type IIS restriction enzymes can be found on New England Biolab's catalog.

An alternative method to carry out adaptor removal reaction can be through the use of deoxyuridine (dU). For example, the last bases of [F_{B}} and [R_{A}*} can be designed to be T. A version of [F_{B}} of [R_{A}*} primers where all the dT base are replaced with dU bases (hereby called ‘dU-laden’ primer) can be used to amplify 112. Following this reaction, the USER enzyme mix (available from NEB Biolabs) can be used to remove the [F_{B}} and [R_{A}*} domains from 112, leaving 3′ overhangs. Next, a 3′-to-5′ ssDNA-specific exonuclease (e.g., Exonuclease I or Exo I) can be used to degrade these 3′ overhangs and form blunt ends.

An example method can comprise providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of a jth polynucleotide when i≠j. Next, a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j can be provided. Next, the first mixture and the second mixture can be contacted to generate a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, and wherein, for each i, the ZipAᵢ sequence specifically links to the ZipBᵢ sequence. Next, optionally, within the third mixture, for each i, a free 3′ end of the ZipAᵢ sequence or the ZipBᵢ sequence can be extended using the ith polynucleotide from the first mixture or the second mixture as a template to generate the ith polynucleotide comprising the SeqAᵢ sequence, the SeqBᵢ sequence and the ZipABᵢ sequence. Next, optionally, within the third mixture, a sequence segment from 3′ and/or 5′end of the ith polynucleotide can be removed (e.g., adaptor removal reaction). Next, optionally, within the third mixture, the ith polynucleotide comprising the SeqAᵢ sequence, the SeqBᵢ sequence and the ZipABᵢ sequence can be circularized to generate a circularized polynucleotide. Next, optionally, within the third mixture, the circularized polynucleotide can be linearized such that, for each i, the ZipABᵢ sequence is not flanked by the SeqAᵢ sequence and the SeqBᵢ sequence. In some cases, a fourth mixture of at least n polynucleotides can be provided, wherein an ith polynucleotide comprises a SeqCᵢ sequence and a ZipCᵢ sequence, and wherein the ZipCᵢ sequence is different from a ZipCⱼ sequence of a jth polynucleotide when i≠j. Next, the third mixture and the fourth mixture can be contacted to generate a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein, for each i, the ZipCᵢ sequence specifically links to the ZipABᵢ sequence. The methods can be repeated to link additional fragments to synthesize the polynucleotides of interest.

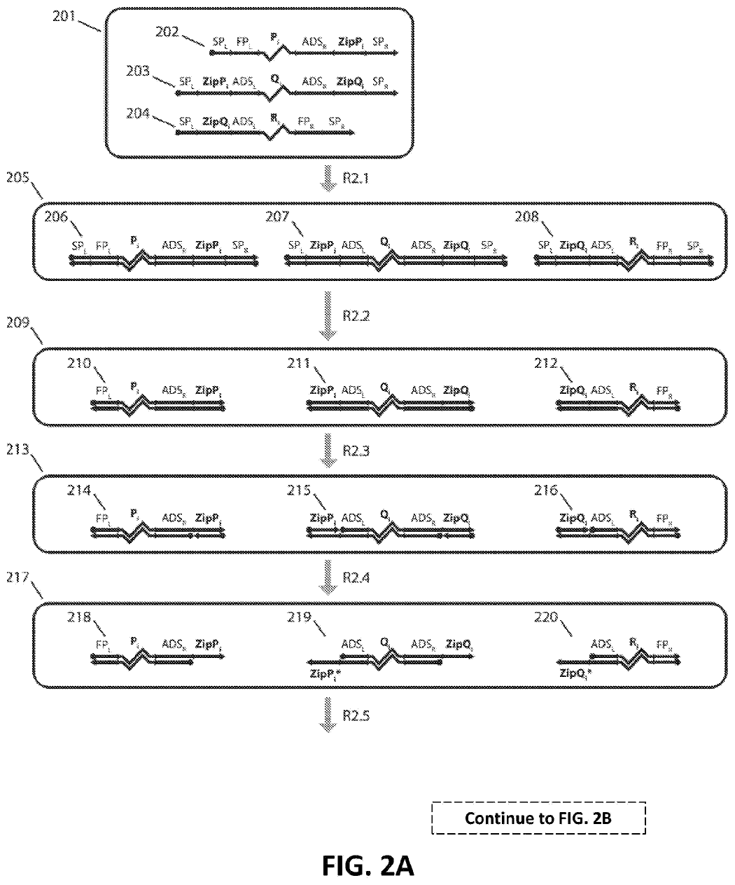

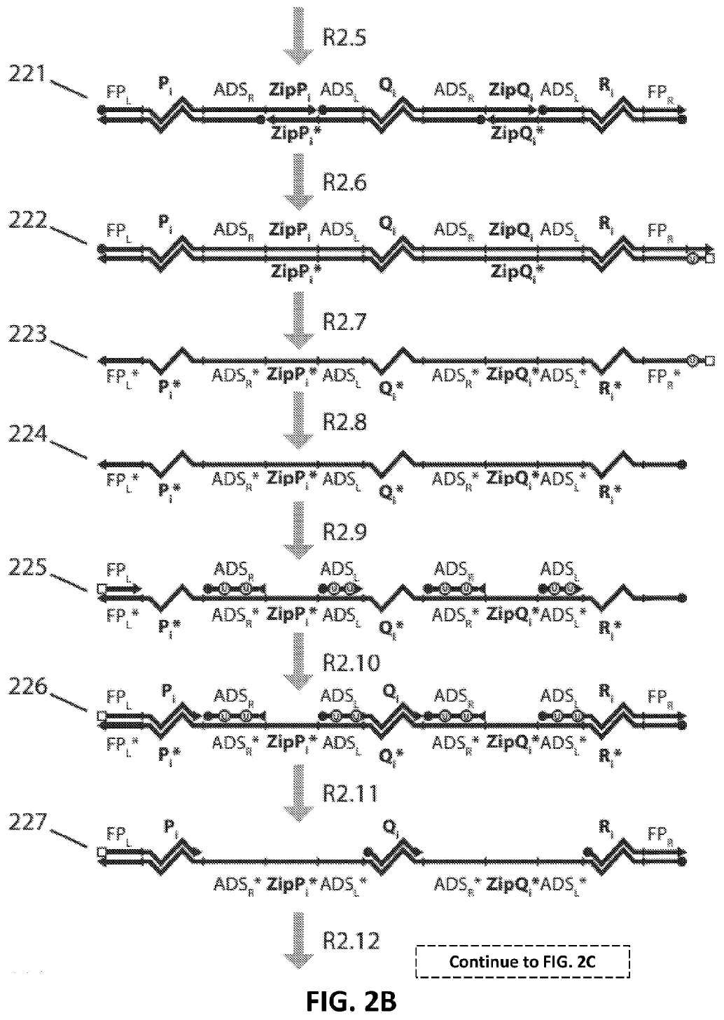

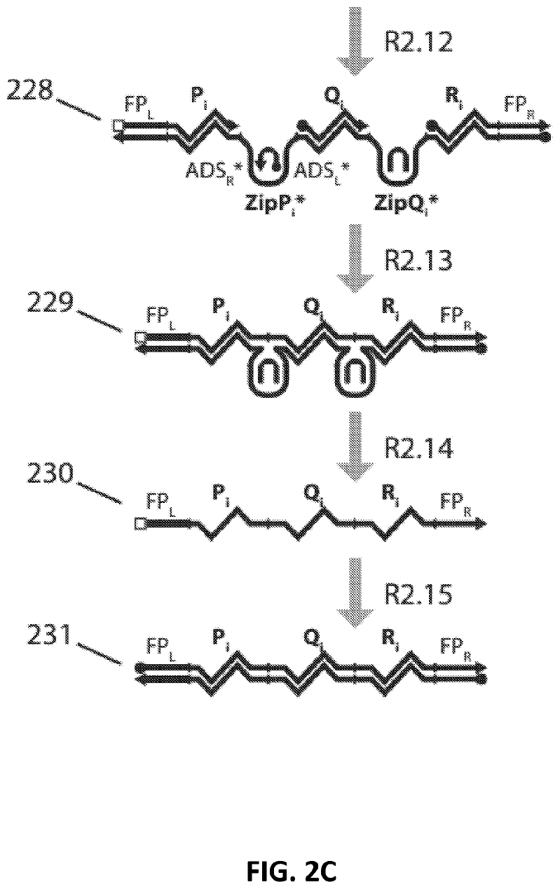

The present disclosure, in some other aspects, provides methods of synthesizing a plurality of n polynucleotides from two or more fragments in a single mixture. The method can comprise providing a mixture comprising a first subpopulation of n polynucleotides, a second subpopulation of n polynucleotides, and a third subpopulation of n polynucleotides. The first subpopulation, the second subpopulation, and the third subpopulation can be mixed within a single mixture. In another words, for each polynucleotide of the plurality of polynucleotides to be synthesized, three or more nucleic acid fragments can be assembled in a single mixture without contacting a first mixture with a second mixture first. In the first subpopulation, an ith polynucleotide can comprise a SeqAᵢ sequence and a ZipAᵢ sequence. The ZipAᵢ sequence can be different from a ZipA sequence when i≠j. In the second subpopulation, an ith polynucleotide can comprise a SeqBᵢ sequence and a ZipBᵢ sequence. The ZipBᵢ sequence can be different from a ZipBᵢ sequence when i≠j. In the third subpopulation, an ith polynucleotide can comprise a SeqCᵢ sequence and a ZipCᵢ sequence. The ZipCᵢ sequence can be different from a ZipCᵢ sequence when i≠j. Next, the first subpopulation, the second subpopulation and the third subpopulation can be contacted within the mixture to generate a plurality of n polynucleotides, where an ith polynucleotide of the plurality comprises the Seqᵢ sequence comprising the SeqAᵢ sequence, the SeqBᵢ sequence and the SeqCᵢ sequence. The SeqAᵢ sequence, the SeqBᵢ sequence and the SeqCᵢ sequence of the Seqᵢ sequence can be linked seamlessly. The Seqᵢ sequence can comprise the SeqAᵢ sequence and the SeqBᵢ sequence without an intervening sequence in between the SeqAᵢ sequence and the SeqBᵢ sequence. The Seqᵢ sequence can comprise the SeqBᵢ sequence and the SeqCᵢ sequence without an intervening sequence in between the SeqBᵢ sequence and the SeqCᵢ sequence. The Seqᵢ sequence can comprise the SeqAᵢ sequence, the SeqBᵢ sequence and the SeqCᵢ sequence sequentially from the 5′ end to the 3′ end. For example, as shown in FIG. 2A and FIG. 2B, three mixtures can be contacted in a single mixture to generate a plurality of polynucleotides of interest.

In some cases, in the second subpopulation, the ZipBᵢ sequence can be a ZipB1ᵢ sequence, and the ith polynucleotide can further comprise a ZipB2ᵢ sequence. The two connector sequences can be located at various positions. The two connector sequences may not flank the sequence of interest. For example, in some cases, the SeqBᵢ sequence can be located in between the ZipB1ᵢ sequence and the ZipB2ᵢ sequence. In some cases, the ZipB1ᵢ sequence can be located in between the SeqBᵢ sequence and the ZipB2ᵢ sequence. In some cases, the ZipB2ᵢ sequence can be located in between the SeqBᵢ sequence and the ZipB1ᵢ sequence.

The ZipAᵢ sequence and the ZipBᵢ sequence can be connector sequences for linking the SeqAᵢ sequence and the SeqBᵢ sequence. The ZipB2ᵢ sequence and the ZipCᵢ sequence can be connector sequences for linking the SeqBᵢ sequence and the SeqCᵢ sequence. In some cases, for each i, the ZipAᵢ sequence and the ZipBᵢ sequence are a same nucleic acid sequence. In some cases, for each i, the ZipAᵢ sequence and the ZipBᵢ sequence are complementary. In some cases, for each i, the ZipAᵢ sequence and the ZipBᵢ sequence are different nucleic acid sequences. In some cases, for each i, the ZipB2ᵢ sequence and the ZipCᵢ sequence are a same nucleic acid sequence. In some cases, for each i, the ZipB2ᵢ sequence and the ZipCᵢ sequence are complementary. In some cases, for each i, the ZipB2ᵢ sequence and the ZipCᵢ sequence are different nucleic acid sequences. For each i, the ZipAᵢ sequence, the ZipBᵢ sequence, the ZipB1ᵢ sequence, the ZipB2ᵢ sequence, or the ZipCᵢ sequence can be of various length, for example, from 5 nucleotides to 200 nucleotides in length. For each i, the SeqAᵢ sequence, the SeqBᵢ sequence, or the SeqCᵢ sequence can be from 5 nucleotides to 5,000 nucleotides in length.

The method can further comprise linking (i) the ZipAᵢ sequence and the ZipB1ᵢ sequence, and/or (ii) the ZipB2ᵢ sequence and the ZipCᵢ sequence. In some cases, linking comprising hybridizing (i) the ZipAᵢ sequence and the ZipB1ᵢ sequence, and/or (ii) the ZipB2ᵢ sequence and the ZipCᵢ sequence. For example, the ZipAᵢ sequence and the ZipB1ᵢ sequence can be complementary, and the ZipB2ᵢ sequence and the ZipCᵢ sequence can be complementary. In some cases, a plurality of intermediate products can be generated, where an ith intermediate product of the plurality comprises the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence. The ith intermediate product of the plurality can comprise the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence sequentially from 5′ end to 3′ end (e.g., 221-227 of FIG. 2B). The method can further comprise removing the ZipAᵢ sequence (or the ZipB1ᵢ sequence) and the ZipCᵢ sequence (or the ZipB2ᵢ sequence), thereby generating the Seqᵢ sequence comprising the SeqAᵢ sequence, the SeqBᵢ sequence and the SeqCᵢ sequence without any intervening sequence (e.g., see tweezer method in Example 2). In some cases, removing the connector sequences can be conducted by using a DNA tweezer. For example, the ZipAᵢ sequence or the ZipCᵢ sequence region of one strand can be degraded, and a staple strand can be used to hybridize with regions flanking the ZipAᵢ sequence or the ZipCᵢ sequence on the complementary strand to bring the SeqAᵢ sequence, the SeqBᵢ sequence and the SeqCᵢ sequence region in close proximity for ligation.

In various embodiments described herein, the plurality of polynucleotides synthesized herein can be a functional genetic element. For example, the concatenation of the SeqAᵢ sequence and the SeqBᵢ sequence without an intervening sequence can be a functional genetic element. In some cases, concatenation of the SeqAᵢ sequence, the SeqBᵢ sequence and the SeqCᵢ sequence without any intervening sequence can be a functional genetic element. The sequence of the functional genetic element can exist nationally in a cell or tissue. The functional genetic element can be a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, a primer-extension gRNA for prime editing, or any combination thereof. It is to be understood that the methods described herein may be used to assemble any genetic element or any polynucleotide of interest. In some cases, the polynucleotide of interest may not be functional or be a functional element. The functional genetic element may not comprise a sequence that is identical to any connector sequence or Zip sequence described herein. The connector sequence or Zip sequence can be irrelevant to any polynucleotides of interest synthesized herein. For example, the functional genetic element may not comprise a sequence that is identical to the ZipAᵢ sequence, the ZipBᵢ sequence, the ZipABᵢ sequence or the ZipCᵢ sequence. For each i, the SeqAᵢ sequence, the SeqBᵢ sequence, and/or the SeqCᵢ sequence can be uniquely or specifically linked. In some cases, a plurality of at least 2, 10, 20, 30, 40, 50, 60, 70, 80, 90, 100, 500, 1,000, 5,000 or more polynucleotides can be synthesized in one mixture.

The polynucleotide synthesized can be of various length. For example, polynucleotide of interest can be at least about 15, 20, 25, 30, 35, 40, 45, 50, 55, 60, 65, 70, 75, 80, 85, 90, 95, 100, 150, 200, 300, 400, 500, 600, 700, 800, 900, 1,000, 2,000, 3,000, 4,000, 5,000, 6,000, 7,000, 8,000, 9,000, 10,000, 15,000, 20,000, 50,000, 100,000 or more nucleotides in length. In some cases, each polynucleotide of the plurality synthesized can be from about 15 to about 15,000 nucleotides in length.

The present disclosure, in some aspects, provides methods for synthesizing a plurality of polynucleotides (e.g., a plurality of at least n polynucleotides, where n is an integer equal to or greater than 2), where an ith polynucleotide of the plurality comprises a Seqᵢ sequence (where i=1 to n), and the Seqᵢ sequence comprises a SeqAᵢ sequence and a SeqBᵢ sequence. In the methods provided herein, an ith polynucleotide of the plurality can comprise a ZipABᵢ sequence, a SeqAᵢ sequence and a SeqBᵢ sequence sequentially from 5′ end to 3′ end. For example, the methods provided herein can comprise providing a first mixture of n polynucleotide. In the first mixture, an ith polynucleotide can comprise a SeqAᵢ sequence and a ZipAᵢ sequence. For each i, the ZipAᵢ sequence can be unique within the first mixture. In other words, the ZipAᵢ sequence can be different from a ZipAⱼ sequence when i≠j. For example, ZipA₁ sequence can be different from a ZipA₂ sequence within the first mixture. Next, a second mixture of n polynucleotides can be provided. In the second mixture, an ith polynucleotide can comprise a SeqBᵢ sequence and a ZipBᵢ sequence. For each i, the ZipBᵢ sequence can be unique within the second mixture. In other words, the ZipBᵢ sequence can be different from a ZipBᵢ sequence when i≠j. Next, the first mixture and the second mixture can be contacted to generate a third mixture of a plurality of n polynucleotides, where an ith polynucleotide can comprise a ZipABᵢ sequence, a SeqAᵢ sequence and a SeqBᵢ sequence sequentially from 5′ end to 3′ end. In the third mixture, for each i, the ZipABᵢ sequence can be unique. The ZipABᵢ sequence can be different from a

ZipABᵢ sequence when i≠j. In the methods provided herein, the SeqAᵢ sequence and the SeqBᵢ sequence can be linked without an intervening sequence (e.g., no Zip sequences or other sequences in between). The SeqAᵢ sequence and the SeqBᵢ sequence can be linked seamlessly. For each i, the ZipAᵢ sequence and the ZipBᵢ sequence can be connector sequences for linking the SeqAᵢ sequence and the SeqBᵢ sequence. As described herein, in some cases, generating the third mixture can comprise linking, for each i, the ZipAᵢ sequence and the ZipBᵢ sequence. In some cases, linking can comprise hybridizing, for each i, the ZipAᵢ sequence and the ZipBᵢ sequence. The methods can further comprise extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence. In some cases, an intermediate product comprising the SeqBᵢ sequence, the ZipABᵢ sequence and the SeqAᵢ sequence sequentially from 5′ end to 3′ end (e.g., 111, 112 and 113 of FIG. 1) can be generated. The methods can further comprise adapter removal, circularization, and/or linearization. Optionally, the third mixture can be contacted with a fourth mixture to link further nucleic acid fragments onto the synthesized Seqᵢ sequence comprising the SeqAᵢ sequence and the SeqBᵢ sequence. An ith polynucleotide of the fourth mixture can comprise a SeqCᵢ sequence and a ZipCᵢ sequence, and the ZipCᵢ sequence can be different from a ZipCⱼ sequence when i≠j. In such cases, a fifth mixture of n polynucleotides can be generated, where an ith polynucleotide can comprise the Seqᵢ sequence comprising the SeqAᵢ sequence and the SeqBᵢ sequence and further comprising the SeqCᵢ sequence. In some cases, the SeqBᵢ sequence and the SeqCᵢ sequence can be linked without an intervening sequence. FIG. 1 provides an example of the methods described herein.

Any nucleic acid molecule described in the present disclosure can be a double-stranded nucleic acid molecule or single-stranded nucleic acid molecule. In some cases, a nucleic acid molecule may comprise a double-stranded region and a single-stranded region. For example, the nucleic acid molecule having a connector sequence or anti-connector sequence may be a double-stranded nucleic acid molecule having the connector sequence or anti-connector sequence region as a single-stranded region (e.g., an overhang or sticky end). The overhang can be at least 1, 2, 3, 4, 5, 6, 7, 8, 9, 10, or more nucleotides long. The overhang can be at 5′ end or 3′ end of a nucleic acid molecule.

Any nucleic acid molecule describe herein can comprise one or more modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs can be compatible with natural and mutant polymerases for de novo and/or amplification synthesis.

### Compositions for Synthesizing Polynucleotides

The present disclosure also compositions for synthesizing the polynucleotides of interest. For example, a composition provided herein can comprise any mixture described herein, including the first mixture, the second mixture, and the third mixture described herein. For another example, the composition provided herein can comprise an intermediate product or a mixture of intermediate products generated during the process of synthesizing the final products of interest.

In some cases, provided herein is a composition for synthesizing a plurality of n polynucleotides. The composition can comprise a first mixture of n polynucleotides. An ith polynucleotide of the first mixture can comprise a SeqAᵢ sequence and a ZipAᵢ sequence (where i=1 to n). The ZipAᵢ sequence can be different from a ZipAᵢ sequence when i≠j. The composition can further comprise a second mixture of n polynucleotides. An ith polynucleotide can comprise a SeqBᵢ sequence and ZipBᵢ sequence (where i=1 to n). The ZipBᵢ sequence can be different from a ZipBj sequence when i≠j. In the composition, for each of i, concatenation of the SeqAᵢ sequence and the SeqBᵢ sequence without intervening sequence can be a functional genetic element. The ZipAᵢ sequence and the ZipBᵢ sequence can be connector sequences for linking the SeqAᵢ sequence and the SeqBᵢ sequence. The ZipAᵢ sequence and the ZipBᵢ sequence can be connector sequences for linking the SeqAᵢ sequence and the SeqBᵢ sequence specifically.

The first mixture and the second mixture can be within a same compartment or a same mixture. The first mixture and the second mixture can be combined or mixed to form a single mixture. The ZipAᵢ sequence and the ZipBᵢ sequence can be linked. The ZipAᵢ sequence and the ZipBᵢ sequence can be hybridized. The ZipAᵢ sequence and the ZipBᵢ sequence can be a same nucleic acid sequence, complementary nucleic acid sequences, or different nucleic acid sequences. The composition can further comprise a third mixture of n polynucleotides, where an ith polynucleotide comprises a SeqCᵢ sequence and a ZipCᵢ sequence and the ZipCᵢ sequence is different from a ZipCᵢ sequence when i≠j. In the compositions, for each of i, concatenation of the SeqAᵢ sequence, the SeqBᵢ sequence, and the SeqCᵢ sequence without any intervening sequence can be a functional genetic element. The ZipCⱼ sequence can be a connector sequence for linking the SeqCᵢ sequence and a sequence comprising the SeqAᵢ sequence and the SeqBᵢ sequence. The ZipCᵢ sequence can be a same nucleic acid sequence as the ZipAᵢ sequence or the ZipBᵢ sequence. The ZipCⱼ sequence can be a different nucleic acid sequence from the ZipAᵢ sequence or the ZipBᵢ sequence. The ZipCⱼ sequence, the ZipAᵢ sequence and the ZipBᵢ sequence can be a same nucleic acid sequence. The first mixture, the second mixture and the third mixture can be within a same compartment or a same mixture. The functional genetic element can be a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, or a primer-extension gRNA for prime editing.

The present disclosure, in some aspects, provides a composition comprising a polynucleotide having at least two double-stranded regions separated by a single-stranded region (see e.g., 228 of FIG. 2C). The at least two double-stranded regions (e.g., Pᵢ and Qᵢ) can comprise a first double-stranded region (e.g., Pᵢ) and a second double-stranded region (Qᵢ). The single-stranded region (e.g., ADS_{R}*, ZipPᵢ*, and ADS_{L}*) can hybridize with a stable strand such that the single-stranded region forms a loop stabilized by the stable strand to bring a 3′ end of the first double-stranded region and a 5′ end of the second double-stranded region to close proximity. The first double-stranded region and the second double-stranded region can be from a same functional genetic element. The functional genetic element can comprise a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, and/or a primer-extension gRNA for prime editing. The 3′ end of the first double-stranded region and the 5′ end of the second double-stranded region can be in close proximity for ligation. The 3′ end of the first double-stranded region and the 5′ end of the second double-stranded region can be joined. The 3′ end of the first double-stranded region and the 5′ end of the second double-stranded region can be ligated (e.g., using T4 DNA ligase, T7 DNA ligase, or T3 DNA ligase). The single-stranded region can comprise, from 5′ to 3′, a first segment and a second segment, and the stable strand can comprise, from 5′ to 3′, a third segment and a fourth segment. To form the loop structure, the first segment can hybridize with the third segment and the second segment can hybridize with the fourth segment. In some cases, the composition provided herein can comprise a plurality of polynucleotides, each having at least two double-stranded regions separated by a single-stranded region, wherein the at least two double-stranded regions comprise a first double-stranded region and a second double-stranded region, wherein the single-stranded region hybridizes with a stable strand such that the single-stranded region forms a loop stabilized by the stable strand to bring a 3′ end of the first double-stranded region and a 5′ end of the second double-stranded region to close proximity, and wherein the first double-stranded region and the second double-stranded region are from a same functional genetic element. In such cases, each polynucleotide of the plurality may be a different functional genetic element. The polynucleotide can comprise three double-stranded regions separated by two single-stranded regions. Each single-stranded region can hybridize with a stable strand. The three double-stranded regions can be from a same functional genetic element.

### Nucleic Acid Fragments

A plurality of polynucleotides (e.g., a plurality of at least n polynucleotides, where n is equal to or greater than 2) of interest can be synthesized or assembled by the methods described herein. The plurality of polynucleotides of interest can be functional genetic elements. The functional genetic element can be a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, or a primer-extension gRNA for prime editing. The plurality of polynucleotides of interest can be synthesized or assembled using two or more nucleic acid fragments. The plurality of polynucleotides of interest can be synthesized or assembled using two or more nucleic acid fragments in a same mixture or a single mixture. In some cases, two or more different polynucleotides can be synthesized or assembled together in the same mixture. Each polynucleotide of the plurality of polynucleotides of interest can be synthesized or assembled from two or more nucleic acid fragments, where each nucleic acid fragment can be from a different mixture. When combining two or more different mixtures containing two or more different nucleic acid fragments into a single mixture, various reactions can be performed to generate the synthesized polynucleotides. The plurality of polynucleotides of interest can be a plurality of different mutants or variants of a wild-type polynucleotide. For example, a mixture of 100 different polynucleotides can be synthesized in a same mixture, where each polynucleotide comprises a mutation (e.g., a point mutation, a deletion, an addition, or a modification) of a wild-type sequence or a reference sequence.

As described herein, a given polynucleotide of the plurality being synthesized can be referred to as an ith polynucleotide which may comprise a sequence referred to as “Seqᵢ sequence” (where i=1 to n). For example, the given polynucleotide can be a first polynucleotide comprising a Seqi sequence, a second polynucleotide comprising a Seq₂ sequence, a third polynucleotide comprising a Seq₃ sequence . . . or an nth polynucleotide comprising a Seqᵢ sequence. For each given nucleotide, the Seq sequence can be synthesized or assembled by two or more nucleic acid fragments specifically. For example, the Seq sequence can be synthesized or assembled by SeqAᵢ SeqBᵢ SeqCᵢ SeqD or more nucleic acid fragments. In some cases, the plurality of nucleic acid fragments containing SeqA sequences (e.g., SeqA₁, SeqA₂, SeqA₃ . . . and/or SeqBₙ) can be provided in a first mixture. The nucleic acid fragments containing SeqA sequences can be a family of polynucleotides. In some cases, the plurality of nucleic acid fragments containing SeqB sequences (e.g., SeqB₁, SeqB₂, SeqB₃ . . . and/or SeqBₙ) can be provided in a second mixture. The nucleic acid fragments containing SeqB sequences can be a family of polynucleotides. In some cases, the plurality of nucleic acid fragments containing SeqC sequences (e.g., SeqC₁, SeqC₂, SeqC₃ . . . and/or SeqCₙ) can be provided in a third mixture. The nucleic acid fragments containing SeqC sequences can be a family of polynucleotides.

A nucleic acid fragment for synthesizing or assembling a polynucleotide of interest described herein can further comprise a connector or a Zip sequence. Within each mixture of nucleic acid fragments, the Zip sequence in a given fragment containing a given SeqA sequence is unique such that a given SeqA sequence is specifically or uniquely linked to another fragment containing a SeqB sequence from another mixture when the two mixtures are combined. For example, in various embodiments, a first mixture of n polynucleotides can be provided, where an ith polynucleotide comprises a SeqAᵢ sequence and a ZipAᵢ sequence, and where the ZipAᵢ sequence is different from a ZipA sequence when i≠j. For another example, in various embodiments, a second mixture of n polynucleotides can be provided, where an ith polynucleotide comprises a SeqBᵢ sequence and ZipBᵢ sequence, and wherein the ZipBᵢ sequence is different from a ZipBᵢ sequence when i≠j. In various embodiments, a SeqAᵢ sequence, a SeqBᵢ sequence, a SeqCᵢ sequence, or more sequences can be specifically linked to form the functional genetic element of interest. In other words, the SeqAᵢ sequence, the SeqBᵢ sequence, the SeqCᵢ sequence, or more sequences can be derived from a same functional genetic element. The nucleic acid fragment described herein can comprise a restriction enzyme recognition site. For example, the restriction enzyme recognition site can be a recognition site for Type IIS restriction enzyme. Examples of Type-IIS restriction enzymes which can be useful in the present disclosure include, but are not limited to, EarI, MnlI, PleI, AlwI, BbsI, BbvI, BcoDI, BsaI, BseRI, BsmAI, BsmBI, BspMI, Esp3I, HgaI, SapI, SfaNI, BbvI, BsmFI, BsrDI, BtsI, FokI, BseRI, HphI, MlyI and MboII. In some cases, two or more different restriction enzymes can be used during nucleic acid construction process. In some cases, a restriction enzyme that create a 4-bp 5′ overhang (for example, BbsI, BbvI, BcoDI, Bsal, BsmBI, FokI, etc.) can be used. In some cases, a restriction enzyme that creates a blunt end or 3′ overhang (for example, BseRI, BsrDI, BtsI, MlyI, etc.) can be used.

A nucleic acid fragment described herein can be circularized. In some cases, a nucleic acid fragment generated as an intermediate product can be circularized. For example, the nucleic acid fragment can be circularized by joining two ends of the nucleic acid fragment by ligation. The ligation can be blunt end ligation. The ligation can be performed after creating sticky ends using 5′-to-3′ exonuclease (e.g., Gibson Assembly), 3′-to-5′ exonuclease (e.g., sequence and ligase independent cloning or SLIC), or USER enzyme mix (e.g., USER friendly DNA recombination or USERec). Additional examples of circularization methods include, but are not limited to, circular polymerase extension cloning (CPEC) and seamless ligation cloning extract (SLICE) assembly. Alternatively, these two ends can be joined by overlapping PCR. A variety of ligases can be used for ligation, for example, including but not limited to, T4 DNA ligase, T4 RNA ligase, E. coli DNA ligase.

The nucleic acid fragment can be synthesized chemically. For example, the initial mixtures used to synthesize or assemble any polynucleotide of interest can be synthesized chemically. For example, the nucleic acid fragment can be pre-synthesized by chip-based synthesis. In some cases, the nucleic acid fragment synthesized can be equal to or greater than about 10, 20, 30, 40, 50, 60, 70, 80, 90, 100, 150, 200, 250, 300, 350, 400, or more nucleotides in length. In some cases, the nucleic acid fragment synthesized by can be equal to or less than about 500, 450, 400, 350, 300, 250, 200, 150, 100, 90, 80, 70, 60, 50, 40, 30, 20, or 10 nucleotides in length. For example, in some cases, the nucleic acid fragments can be at least about 5, 10, 15, 20, 25, 30, 35, 40, 45, 50, 55, 60, 65, 70, 75, 80, 85, 90, 95, 100, 150, 200, 300, 400, 500, 600, 700, 800, 900, 1,000, 2,000, 3,000, 4,000, 5,000, 6,000, 7,000 8,000, 9,000, 10,000 or more nucleotides in length. The nucleic acid fragments can be from 5to 50, from 5 to 100, from 5 to 200, from 5 to 500, from 5 to 1,000, from 5 to 2,000, from 5 to 5,000, from 5 to 10,000, from 5 to 50,000, from 10 to 200, from 10 to 500, from 10 to 1,000, from 10 to 5,000, from 10 to 10,000, from 100 to 1,000, from 200 to 5,000, or from 200 to 10,000 nucleotides in length. For example, in some cases, for each i, the SeqAᵢ sequence, the SeqBᵢ sequence, or the SeqCᵢ sequence is from 5 nucleotides to 5,000 nucleotides in length.

In various embodiments, the nucleic acid fragment containing the SeqAᵢ sequence, the nucleic acid fragment containing the SeqBᵢ sequence, the nucleic acid fragment containing the SeqCᵢ sequence or more fragments can be pre-synthesized chemically and provided in a single pool. A first mixture of nucleic acid fragments containing the SeqA sequences, a second mixture of nucleic acid fragments containing the SeqB sequences, or the third mixture of nucleic acid fragments containing the SeqC sequences can be prepared from the single pool, for example, by specifically amplifying the fragments containing the SeqA sequences, the SeqB sequences or the SeqC sequences. For example, FIG. 1 shows an example of preparing the mixtures 107, 110 or 120 from the pool 101. For example, in some cases, prior to providing a mixture of nucleic acid fragments, a family of oligonucleotides can be amplified (e.g., using PCR) from a single pool of oligonucleotides pre-synthesized to contain two or more families of oligonucleotides (e.g., 102, 103, and 104). The family of oligonucleotides (e.g., 102) can be amplified using primers specific for the Operator sequences flanking the Product Constituent sequences and the Zip sequences to generate a mixture of double-stranded nucleic acids (e.g., 105). After amplification, the double-stranded nucleic acids can be treated with enzymes to remove one or more Operator sequences (e.g., adaptor removal reaction or adaptor removal). For example, when performing the amplification, one primer can comprise deoxyuridine nucleotides such that the double-stranded nucleic acids can be treated with USER enzyme and exonuclease to remove the Operator sequence (e.g., F_{A}). The mixture of double-stranded nucleic acids (e.g., 106) can further be treated with an enzyme to generate a mixture of single-stranded nucleic acids (e.g., 107).

As an example, in various embodiments, the methods can further comprise, prior to providing two or more mixtures for polynucleotide assembly, a pool of polynucleotides comprising the at least n polynucleotides of the first mixture (e.g., a first family), the at least n polynucleotides of the second mixture (e.g., a second family), and/or the at least n polynucleotides of the fourth mixture (e.g., a third family) can be provided. Next, the at least n polynucleotides of the first mixture, the at least n polynucleotides of the second mixture, and/or the at least n polynucleotides of the fourth mixture can be amplified from the pool to generate double-stranded polynucleotides. The at least n polynucleotides of the first mixture, the at least n polynucleotides of the second mixture, and/or the at least n polynucleotides of the fourth mixture can be amplified in different reactions using different primers. For example, a pair of primers targeting the primer binding site (e.g., Operator sequence) common to the first family can be used to only amplify the first family of polynucleotides. Next, the Operator sequence (e.g., the primer binding site) can be removed from the double-stranded polynucleotides. Next, one strand of the double-stranded polynucleotides can be removed (e.g., degraded) to generate the at least n polynucleotides of the first mixture, the at least n polynucleotides of the second mixture, and/or the at least n polynucleotides of the fourth mixture.

### Connector Sequences

A connector sequence (also referred to as Zip sequence, or Z for short in some cases) can be used to link (or specifically link) one nucleic acid molecule (or nucleic acid fragment) to another nucleic acid molecule (or nucleic acid fragment). The connector sequence of one nucleic acid molecule can hybridize (e.g., form base pair or base pairs) with an anti-connector sequence (e.g., Zip* sequence or Z*) of another nucleic acid molecule. The anti-connector sequence can be complementary (e.g., fully or substantially complementary) with the connector sequence. The anti-connector sequence can be hybridizable with the connector sequence under certain conditions (e.g., temperature, buffer condition, pH, etc.). The anti-connector sequence can be a reverse complement sequence (or complementary sequence) of the connector sequence. When the connector sequence hybridizes with the anti-connector sequence, the base pair(s) formed can be at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 100, or more base pairs. The base pairs formed between the connector sequence and the anti-connector sequence can be contiguous or non-contiguous. For example, in the cases where non-contiguous base pairs are formed, there may be unpaired region or regions separating paired regions. If a first nucleic acid molecule comprises a connector sequence, then a complementary sequence of the connector sequence on a second nucleic acid molecule can be referred to as an anti-connector sequence. The connector sequence or Zip sequence (or anti-connector sequence or Zip* sequence) described herein can be of various length. For example, the connector sequence or Zip sequence can be at least about 2, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 250, 280, 300, 350, 400, 450 or more nucleotides in length. The connector sequence or Zip sequence can be at most about 500, 450, 400, 350, 300, 250, 200, 150, 100, 50, 20 or less nucleotides in length. The connector sequence or Zip sequence can be from 2 to 50, from 10 to 60, from 5 to 100, from 10 to 200, from 2 to 100, from 5 to 200, from 5 to 300, or from 5 to 400 nucleotides in length. For another example, the connector sequence (or anti-connector sequence) can be greater than or equal to about 2, 3, 4, 5, 6, 7, 8, 9, 10, 11, 12, 13, 14, 15, 20, 25, 30, 35, 40, 45, 50, 55, 60, 65, 70, 75, 80, 85, 90, 100, or more nucleotides in length. The connector sequence (or anti-connector sequence) can be less than or equal to about 300, 250, 200, 150, 100, 90, 85, 80, 75, 70, 65, 60, 55, 50, 45, 40, 35, 30, 25, 20, 15, 14, 13, 12, 11, 10, 9, 8, 7, 6, 5, 4, 3 or 2 nucleotides in length. The connector sequence (or anti-connector sequence) can be at 5′ end or 3′ end of a nucleic acid molecule. The connector sequence (or anti-connector sequence) can also be an internal sequence of a nucleic acid molecule. For example, the connector sequence can be an internal connector sequence and can be exposed at 5′ end or 3′ end by cutting an internal sequence (e.g., a sequence adjacent to the internal connector sequence) of the nucleic acid molecule.

The connector sequence or Zip sequence described herein can be irrelevant to any polynucleotides of interest synthesized herein. The connector sequence or Zip sequence described herein can be arbitrary or predesigned sequences. The functional genetic element may not comprise a sequence that is identical to any connector sequence or Zip sequence described herein. After synthesizing or assembling the polynucleotides containing the final sequences of interest, any connector sequences or Zip sequences can be removed from the polynucleotides to generate the final polynucleotides of interest.

In various embodiments, a first mixture (e.g., a first family) of n polynucleotides can be provided, where an ith polynucleotide of the first mixture can comprise a SeqAᵢ sequence and a ZipAᵢ sequence. For each i, the ZipAᵢ sequence can be unique within the mixture. The ZipAᵢ sequence can be different from a ZipA sequence when i≠j. For example, a ZipA₁ sequence can be different from a ZipA₂, ZipA₃, ZipA+ . . . or ZipA₁₀₀ sequence (assuming 100 fragments are within the first mixture in order to synthesize 100 polynucleotides of interest as final products). In some cases, a second mixture of n polynucleotides can be provided, where an ith polynucleotide of the second mixture can comprise a SeqBᵢ sequence and a ZipBᵢ sequence and the ZipBᵢ sequence can be different from a ZipBᵢ sequence when i≠j. A SeqA in the first mixture can be specifically linked to a corresponding SeqB in the second mixture. For each i, the ZipAᵢ sequence and the ZipBᵢ sequence can be a same nucleic acid sequence or different nucleic acid sequences. For each i, the ZipAᵢ sequence and the ZipBᵢ sequence can be complementary. For each i, the ZipAᵢ sequence and the ZipBᵢ sequence can hybridize with each other.

The connector sequences can be re-used in each mixture (e.g., a family of polynucleotides). For example, a set of connector sequences in the first mixture can be the same as the set of connector sequences in the second mixture. For example, when contacting the first mixture and the second mixture, a third mixture of polynucleotides may be generated, where an ith polynucleotide comprises a SeqAᵢ sequence, a SeqBᵢ sequence and a ZipABᵢ sequence and the ZipABᵢ sequence is different from a ZipABᵢ sequence when i≠j. In some cases, the ZipABᵢ sequence can be the same as the SeqAᵢ sequence or the SeqBᵢ sequence. In some cases, the ZipABᵢ sequence can be the SeqAᵢ sequence or the SeqBᵢ sequence. In some cases, the ZipABᵢ sequence is the SeqAᵢ sequence or the SeqBᵢ sequence after circularization and linearization to expose the ZipABᵢ sequence at the terminus of a polynucleotide. In some cases, a fourth mixture of polynucleotides can be provided, where an ith polynucleotide can comprise a SeqCᵢ sequence and a ZipCᵢ sequence and the ZipCᵢ sequence is different from a ZipCⱼ sequence when i≠j. The ZipCᵢ sequence can be the same as the ZipABᵢ sequence, which can be the same as the SeqAᵢ sequence or the SeqBᵢ sequence. FIG. 1 and FIG. 5 show examples where a same set of Zip sequences can be re-used in different mixtures. For example, the set of Zip sequences used in mixture 504 can be the same as the set of Zip sequences used in mixture 507 or 509.

Different Zips used in one homogenous assembly reaction may have different length or GC content, but may have similar melting temperature. In some cases, hundreds to thousands of Zip sequences are used in a homogenous assembly reaction. Designing the Zip sequences may follow similar rules as designing primers for PCR reaction, such as: all Zips used in one assembly reaction can have similar melting temperature, a Zip may not form strong hairpin at 5° C. below melting temperature, one Zip may not hybridize strongly to another Zip at 5° C. below melting temperature, one Zip may not hybridize strongly to the complement of another Zip at 5° C. below melting temperature.

To generate a set of 1,000 Zips that can be used in the same assembly reaction, one million random 50-mer sequences can be generated first. Next, a desired melting temperature (e.g., 60° C.) can be chosen. Then, the shortest sub-sequence of each of the 30-mer sequence (starting from the 5′ end) whose melting temperature is above the desired melting temperature can be kept, while the rest of the bases can be removed. The resultant one million sequences (with various length) can be called “trimmed random sequences.” Next, the secondary structure of each trimmed random sequence can be evaluated and ranked based on the Gibbs free energy of the minimum free-energy (MFE) structure at 5° C. below the desired melting temperature. The top 10,000 trimmed random sequences, with the highest (e.g., least negative) Gibbs free energy, can be kept. Each of these kept sequences can be called a Zip candidate. If restriction enzymes are used in the assembly reactions, Zip candidate sequences containing such restriction sites may be removed. Next, each of the Zip candidate can be evaluated based on how strongly it forms primer dimer with all other Zip candidates and their complements. A penalty score can be assigned if a strong primer dimer is formed. The penalty score can be positively correlated with the strength of the primer dimer. The sum of all penalty scores can be the final penalty score for each Zip. The top 3,000 Zip candidates with the lowest final penalty score can be kept which can be called Zip finalists. Then this primer dimer evaluation process can be repeated for the 3,000 finalists to choose the top 1,000 sequences with the lowest final penalty score, which can be used as Zips. A number of web-based and stand-alone software packages such as Primer3, UNAfold, NUPACK, PrimerROC, Pythia, Multiple Primer Analyzer (Thermo Fisher), and OligoEvaluator (Sigma-Aldrich) can be used to implement this process.

### EXAMPLES

### Example 1: Successive Zip-Based Orthogonal Primer-Extensions (Circularization Method)

In this example, how to create 1,000 DNA fragments (with 1,000 desired sequences) from 3,000 short oligos in two successive 1,000-plex primer-extension reactions is demonstrated. The orthogonality of the primer-extension reactions can be ensured by 1,000 well-designed ˜20-nt-long orthogonal sequences (e.g., Zips). The Zips may not appear or be identical to any consecutive region in the desired sequences. The desired sequences can be denoted as [Aᵢ|Bᵢ|Cᵢ}, where the subscript i can be 1 to 1,000. For each DNA fragment, the sequences Aᵢ, Bᵢ and Cᵢ can be contributed by three different oligos. First, 1,000 Zip sequences can be designed using criteria and process described herein. These Zips are named Z₁ through Z₁₀₀₀, where Zᵢ corresponds to Aᵢ, Bᵢ and Cᵢ. A few more domains, which will serve as primer binding domains at various steps can also be designed using the same process. These domains can be referred to as Operator domains or Operators. The Zips and Operators may function at different temperature. For example, Zips may have Tₘ values around 55° C., whereas Operators may have Tₘ values around 65° C. The Operators used in this example include F_{A}, R_{A}, F_{B}, R_{B}, F_{C}, R_{C}, W, X and Y.

As shown in FIG. 1A and FIG. 1B, an oligonucleotide pool (101) containing the following 3 sets of oligos, [F_{A}|Zᵢ|Aᵢ|R_{A}} (102), [F_{B}|Bᵢ|W|Y|Zᵢ|R_{B}} (103), and [F_{C}|Cᵢ|Y|X|Zᵢ|R_{C}} (104) (i=1 to 1000), can be designed and ordered from commercial sources such as Agilent, Twist, or IDT, among others. The subset 102 can be amplified by [F_{A}} and [R_{A}*} (reaction R1) to form a mixture of dsDNA 105. In particular, [R_{A}*} may have phosphorothioate modifications at the first 5 phosphodiesters at the 5′ end of the oligo (e.g., 5′-protected, shown as an open square), so that this oligo and its extension product may be rendered resistant to 5′-to-3′ exonuclease such as lambda exonuclease and T7 exonuclease. On the other hand, [F_{A}}'s sequence may have a dT at its 3′ end this and other dTs in [F_{A}} can be replaced by deoxyuridine (dU). This version of [F_{A}} can be referred to as dU-laden [F_{A}}. The dsDNA pool 105 can be treated with the USER enzyme mix and Exonuclease I (Exo I, see reaction R2) to remove the [F_{A}}:[F_{A}*} domain to form 106. This reaction can be referred to as “adaptor removal reaction” or “adaptor removal” in the present disclosure. dsDNA pool 106 can be further treated with a T7 exonuclease to form ssDNA pool 107 (R3). This reaction can be referred to as “ssDNA generation” in the present disclosure.

The oligo pool 101 can also be amplified with 5′-protected [F_{B}} and dU-laden [R_{B}*} (reaction R4) to form dsDNA pool 108, which can be subject to adaptor removal reaction to form the dsDNA pool 109. Further treatment of 109 with T7 exonuclease (reaction R6) generates ssDNA pool 110. The ssDNA pools 107 and 110 can be mixed at 60° C. in typical PCR buffer (e.g., commercial buffer for Q5 DNA polymerase) for 5 to 10 hours so the matching [Zᵢ} and [Zᵢ*} can hybridize (reaction R7). This reaction can be referred to as “Zip-based hybridization” in the present disclosure. Then, a thermophilic DNA polymerase (e.g., Phusion, Q5, or Taq) can be added to the mixture to extend the 3′ ends of each ssDNA (reaction R8) to form dsDNA pool 112 where the matching [Aᵢ} and [Bᵢ} are brought to one molecule. The dsDNA pool 112 can be PCR-amplified again using dU-laden [F_{B}} and [R_{A}*}, and subject to adaptor removal reaction (reaction R9) to form dsDNA pool 113. This dsDNA pool can be circularized with a blunt-end DNA ligase, such as T4 DNA ligase (reaction R10), to form circular dsDNA pool 114, where [Aᵢ} and [Bᵢ} are seamlessly connected. In some cases, the dsDNA pool 113 may be too short for circularization to occur at high efficiency. In other words, the stiffness of dsDNA may prevent efficient circularization. In such situation, the dsDNA pool 113 can be diluted to 1 to 10 pM, denatured, and circularized using ssDNA ligase such as CircLigase or CircLigase II. In either case, the circularization product can be PCR-amplified dU-laden [Y} and 5′-protected [W*} (reaction R11) to form dsDNA pool 115. This PCR can be referred to as “inside-out PCR” in the present disclosure. The domains W and Y can be understood as “primer binding sites for inside-out PCR.” The domain [Y}:[Y*} can be removed in adaptor removal reaction (reaction R12) to form dsDNA pool 116. A ssDNA generation reaction can be set up to degrade the top strand of 116 (reaction R13) to form ssDNA pool 117, as described above.

In parallel to reactions R1 and R4, the oligo pool 101 can be PCR-amplified with 5′ protected [F_{C}} and dU-laden [R_{C}*} (reaction R14) to form dsDNA pool 118. The PCR product can undergo adaptor removal (reaction R15, to form 119), and ssDNA generation (reaction R16) to form ssDNA pool 120. ssDNA pools 117 and 120 can undergo Zip-based hybridization (reaction R17), followed by primer extension (reaction R18) to form dsDNA pool 122, which can further undergo adaptor removal (reaction R19). The resultant dsDNA pool 123 can be circularized (reaction R20, as in R10), to form circular dsDNA or ssDNA pool 124, which can then be PCR-amplified with [X} and [Y*} to form dsDNA pool 125. It can be seen in 125 that the DNA sequences Aᵢ, Bᵢ and Cᵢ are connected without intervening Zip sequences.

This method can be used to further extend the assembly. For example, sequences Dᵢ and Ei can be both appended with Zᵢ (similar to the design of 102 and 103) and assembled to form dsDNA pool containing [Di|Ei} (similar to 115, except that Zᵢ is downstream of [Dᵢ|Eᵢ}, achievable by placing the primer-binding sites for inside-out PCR downstream of Zᵢ, instead of upstream as in 112). This dsDNA pool can undergo adaptor removal, ssDNA generation and used for Zip-based hybridization with the ssDNA pool derived from 125. As a result, [Aᵢ|Bᵢ|Cᵢ} can be assembled with [Dᵢ|Eᵢ} for form [Aᵢ|Bᵢ|Cᵢ|Dᵢ|Eᵢ}.

In other words, since the assembled dsDNA pools (such as 115 and 125) contain Zips and Operators, they can be further assembled. However, if dsDNA pools without Zip or Operator sequences are desired, they can be easily removed. For example, in the design of oligos 102, an Operator named V can be placed between Zᵢ and Aᵢ. Then dU-laden [V} and [Y*} can be used to amplify 125. The PCR product can then undergo adaptor removal to obtain dsDNA pool containing only [Aᵢ|Bᵢ|Cᵢ} sequences.

### Example 2: DNA Tweezers-Based Zip Removal (Tweezer Method)

The previous Example demonstrates how to assemble Aᵢ and Bᵢ without intervening Zip (e.g., Zᵢ in FIG. 1A and FIG. 1B) using circularization. In some cases, circularization may not be used. For example, when a DNA fragment is too long (e.g., >10 kb), a ligation reaction may favor bi-molecular ligation over uni-molecular circularization. An alternative method can be to remove the intervening Zip using DNA tweezers.

As an example (FIG. 2), three dsDNA pools 210, 211, 212 can be created by the process shown in Example 1. Alternatively, they can be made from ssDNA mixture 201, which contains ssDNA pools 202, 203 and 204. The ssDNA pool 202 has the sequences [SP_{L}|FP_{L}|Pᵢ|ADS_{R}|ZipPi|SP_{R}}. The ssDNA pool 203 has the sequences [SP_{L}|ZipPᵢ|ADS_{L}|Qᵢ|ADS_{R}|ZipQᵢ|SP_{R}}. The ssDNS pool 204 has the sequences [SP_{L}|ZipQᵢ|ADS_{L}|Rᵢ|FP_{R}|SP_{R}}. Among these domains SP_{L}, SP_{R}, FP_{L}, FP_{R}, ADS_{L} and ADS_{R} are Operators. The domains Pᵢ, Qᵢ, Rᵢ (i=1 to 1000) as described herein can be the sequences to be assembled to form [Pᵢ|Qᵢ|Rᵢ}. The domains ZipPᵢand ZipQᵢ can be the Zips used to guide orthogonal hybridizations. Amplification of the ssDNA mixture 201 using dU-laden [SP_{L}} and dU-laden [SP_{R}*} (reactions R2.1) generates PCR product 205 containing dsDNA pools 206, 207 and 208, which can undergo adaptor removal reactions (reactions R2.2) to form dsDNA mixture containing dsDNA pools 210, 211 and 212, respectively.

The sequence of domain ADSR can end with a Nb.BtsI site (GCAGTG). The sequence of domain ADSL can start with the reverse-complement of a Nb.BtsI site (CACTGC). Therefore, Nt.BtsI can be used to treat 209 (reaction R2.3), where 210, 211, and 212 will be nicked to produce 214, 215, and 216, respectively, in mixture 213. This mixture can be heated to ˜75° C. (reaction R2.4), which is above the melting temperature of ZipPᵢand ZipQᵢbut not high enough to melt other double-stranded domains in 213, for ˜5 min to form mixture 217, which contains 218, 219 and 220 (derived from 214, 215 and 216, respectively) so the ZipPᵢon 218, ZipPᵢ* on 219, ZipQᵢon 219, and ZipQᵢ* on 220 are exposed. The melted-off ZipPᵢ* from 213, ZipPᵢfrom 215, ZipQᵢ* from 215, and ZipQᵢfrom 216 (collectively called “melted-off Zips”) are now shown in 217. Then the temperature can be reduced from ˜75° C. to ˜55° C., a temperature at which Zips can stably hybridize, and held for 5 to 10 hours. During this time, while some melted-off Zips may rehybridize back to 218, 219 and 220, the Zips may also guide 218, 219 and 220 to form larger complexes 221 (reaction R2.5). The nicks can be ligated, and the ligation product can be amplified with [FPL} and a modified version of [FP_{R}} (the modification being that 5′-T*T*T*T*T*TTdUdU is appended to the 5′ end of [FP_{R}}, where * designates phosphorothioate and dU designates deoxyuridine) to form dsDNA pool 222 (reaction R2.6), whose bottom strand is 5′ protected but also contains dU bases close to the 5′ end. This PCR product can undergo ssDNA generation reaction (reaction R2.7) to form ssDNA pool 223. USER enzyme mixture can be used to cleave the dU nucleotides in 223 to form 5′ unprotected 224 (reaction R2.8).

Next, 5′-protected [FP_{L}}, dU-laden and 3′-blocked [ADS_{R}} (whose 3′ end is modified with inverted dT), and dU-laden [ADS_{L}} are hybridized onto 224 (reaction R2.9) to form 225. A DNA polymerase without strand-displacement activity, such as PhusionU, can be used to extend each extendable 3′ end (reaction R2.10) to form 226. Then USER enzyme mixture can be used (reaction R2.11) to degrade the [ADS_{R}} and [ADS_{L}}, leaving precise ends at the 3′ end of Pᵢ, 5′ end of Qᵢ, 3′ end of Qᵢ and 5′ end of Rᵢ in 227 (note that the last base of dU laden [ADS_{L}} is dU). Then a staple strand with the sequence of [ADS_{L}|ADS_{R}} will be hybridized onto 227 at ˜70° C. (reaction R2.12) to bring the 3′ end of Pᵢ and 5′ end of Qᵢ to proximity, and to bring the 3′ of Qᵢ and 5′ end or Rᵢ to proximity thus forming 228. Next, T4 DNA ligase can be added to ligate the ends in proximity (reaction R2.13) to form 229.

Next, a mixture of ssDNA-specific and dsDNA-specific 5′-to-3′ exonucleases such as T7 exonuclease and RecJf, respectively, can be used to degrade the bottom strands and the staple strands of 229 (reaction R2.14) to form ssDNA pool 230, which can then be PCR-amplified (reaction R2.15) to from dsDNA pool 231.

It is to be understood that the circularization method and tweezer method can be used in combination. For example, ˜200-nt oligonucleotides can be assembled into ˜1 kb fragments using the circularization method. Then the ˜1 kb fragments can be further assembled into 3-5 kb fragments using the tweezer method.

Example 3: Constructing pools of paired CDR3-J polynucleotides from shorter oligo pools

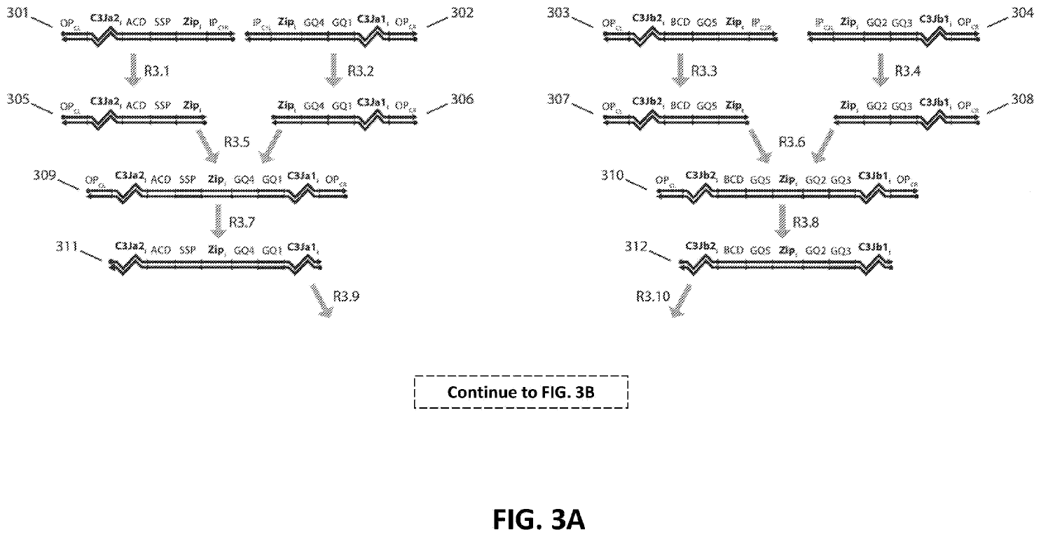

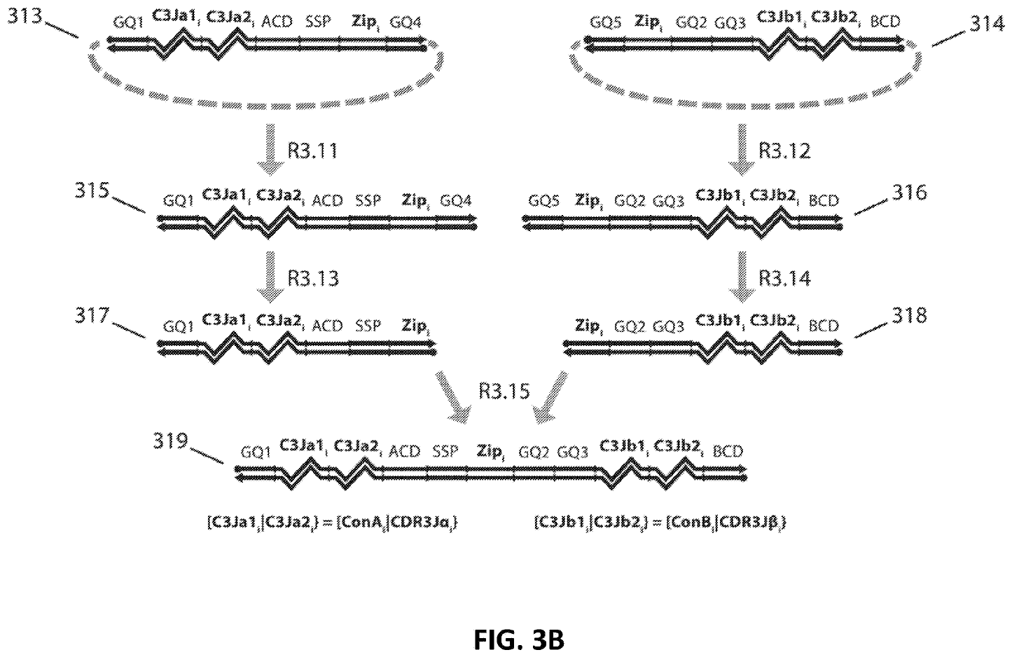

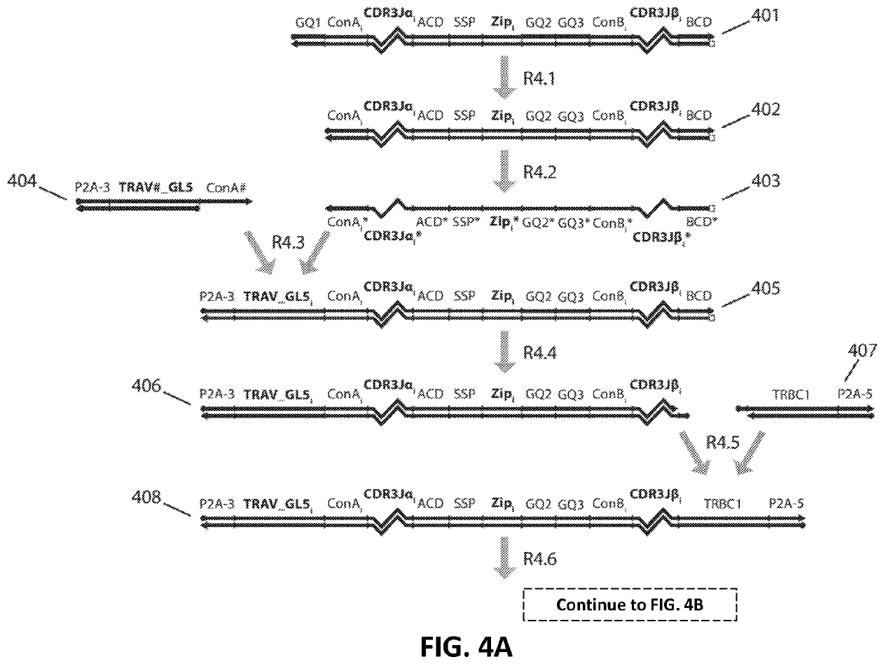

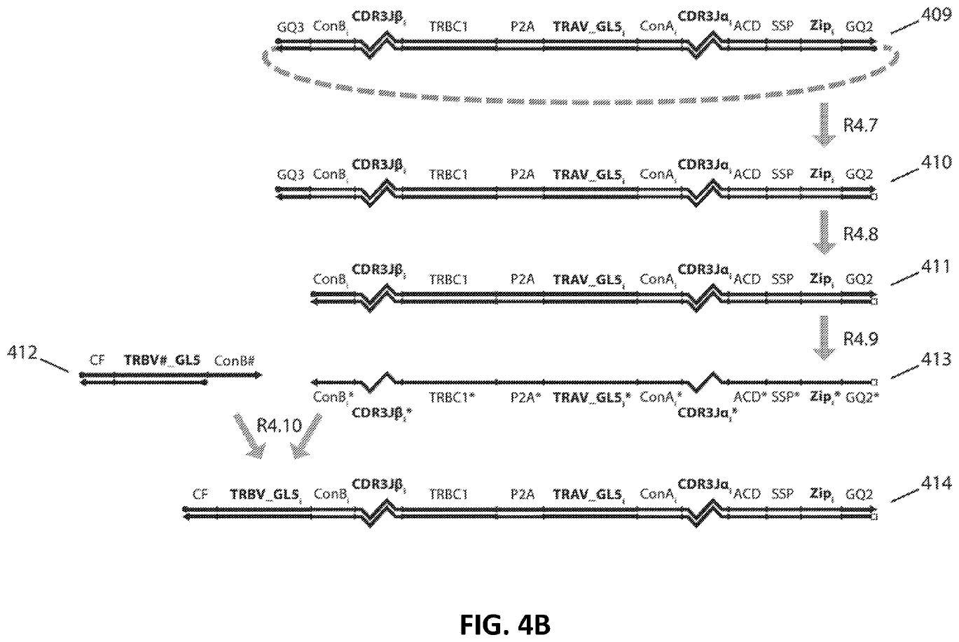

As described in International Application No. PCT/US2020/026558, Chen and Porter disclosed methods to construct thousands of TCR genes in homogenous solutions from pools of paired CDR3-J polynucleotides. Here, this example shows that the paired CDR3-J polynucleotide pool can be assembled from 4 pools of much shorter oligos, in two levels of Zip-based multiplex assemblies where Zips are reused in the 1^{st} and 2^{nd} level (FIG. 3A, FIG. 3B, FIG. 4A and FIG. 4B). This example further shows that the paired CDR3-J polynucleotide pool can be further assembled into full-length TCR genes.

Some of the Zip sequences used in this example are:
5′-CCGAGAGTTTGTTGTCCA-3′
5′-TGCAACAACAGGATCTCC-3′
5′-TCACTTGTTCACCATGGG-3′
5′-GCCTTTGAGCACAAGTGT-3′
5′-CGGTCTGAGACAATTGCA-3′
5′-CGGAGTCAATGTTGGTCA-3′
5′-TGTGTAGGATGTGTTGCC-3′
5′-GCGAGAATCAGTGCATTC-3′
5′-GGTTTTGCTCTGTGTTGC-3′
5′-CGCAGAGTCAATGTGTGT-3′
5′-GCAACAATTCGCCAATCG-3′

The other Zips have the same length and similar GC content.

An oligo pool containing the top or bottom strands of 301, 302, 303 and 304 was obtained from a commercial source. Using this oligo pool as a template:
5′-protected [OP_{CL}} and dU-laden [IP_{C1R}*} were used to amplify and obtain 301,
dU-laden [IP_{C1L}} and 5′-protected [OP_{CR}*} were used to amplify and obtain 302,
5′-protected [OP_{CL}} and dU-laden [IP_{C2R}*} were used to amplify and obtain 303, and
dU-laden [IP_{C2L}} and 5′-protected [OP_{CR}*} were used to amplify and obtain 304.

A total of 583 TCRs were intended to be synthesized. Therefore, each of the family 301, 302, 303, and 304 has 583 species (e.g., sequences).

For simplicity the 5′ protections and dU modifications are not shown in FIG. 3. Four USER-based adaptor removal reactions, R3.1, R3.2, R3.3 and R3.4, were carried out to convert 301, 302, 303 and 304 into 305, 306, 307 and 308, respectively. Next, in step R3.5, the ssDNA generation reaction described in Example 1 was used to generate the top strand of 305 and the bottom strand of 306, which were then mixed to allow oligos in 305 and hybridize to their corresponding oligos in 306 based on complementary Zip sequences (e.g., Zipᵢ and Zipᵢ*). In a reaction analogous to R8 of Example 1, dsDNA pool 309 were prepared. A similar step, R3.6, was carried out to produce 310. The dsDNA pool 309 were amplified with dU-laden [OP_{CL}} and dU-laden [OP_{CR}}, followed to USER-based adaptor removal reaction to produce 311. Through similar steps (R3.8), 310 was converted to 312. Through these processes, each of C3Ja1ᵢ (i=1 to 583) initially carried by dsDNA molecules in 302, is joined with the corresponding C3Ja2ᵢ (i=1 to 583), initially carried by dsDNA molecule in 301, with an intervening sequence that comprises the corresponding Zipᵢ. Similarly, each of C3Jblᵢ(i=1 to 583) initially carried by dsDNA molecules in 304, is joined with the corresponding C3Jb2ᵢ, initially carried by dsDNA molecule in 303, with an intervening sequence that comprises the corresponding Zipᵢ.

The dsDNAs in the pool 311 were then circularized (step R3.9) as described in Example 1 (R10), to form circular DNA pool 313, which was then PCR amplified using primers 5′-protected [GQ1} and dU-laden [GQ4*} (step R3.11) to form dsDNA pool 315. In a similar series of steps (steps R3.10, R3.12), dsDNA pool 312 was converted to circular DNA pool 314, and then amplified to form linear dsDNA pool 316.

It can be seen that, in the pools 313 and 315, each of C3Ja1ᵢ (i=1 to 583) initially carried by dsDNA molecules in 302, is joined with the corresponding C3Ja2i, initially carried by dsDNA molecule in 301, without intervening sequence. Similarly, each of C3Jb1ᵢ (i=1 to 583) initially carried by dsDNA molecules in 304, is joined with the corresponding C3Jb2ᵢ, initially carried by dsDNA molecule in 303, without intervening sequence.

Next, 315 and 316 were converted to 317 and 318 (through steps R3.13 and R3.14), respectively, through adaptor removal reactions. These dsDNA pools underwent ssDNA generation reactions, Zip-based hybridization, and a reaction analogous to R8 of Example 1 to produce dsDNA pool 319. The dsDNA pool 319 was then used as a pool of paired CDR3-J oligos in downstream reactions to assemble full-length TCRs. Note that [C3Jalᵢ|C3Ja2ᵢ} has the sequence of [ConAᵢ|CDR3Jαᵢ}, and [C3Jbli|C3Jb2;} has the sequence of [ConBᵢ|CDR3Jβᵢ}. The latter annotations are useful in understanding the downstream reactions (FIG. 4A and FIG. 4B).

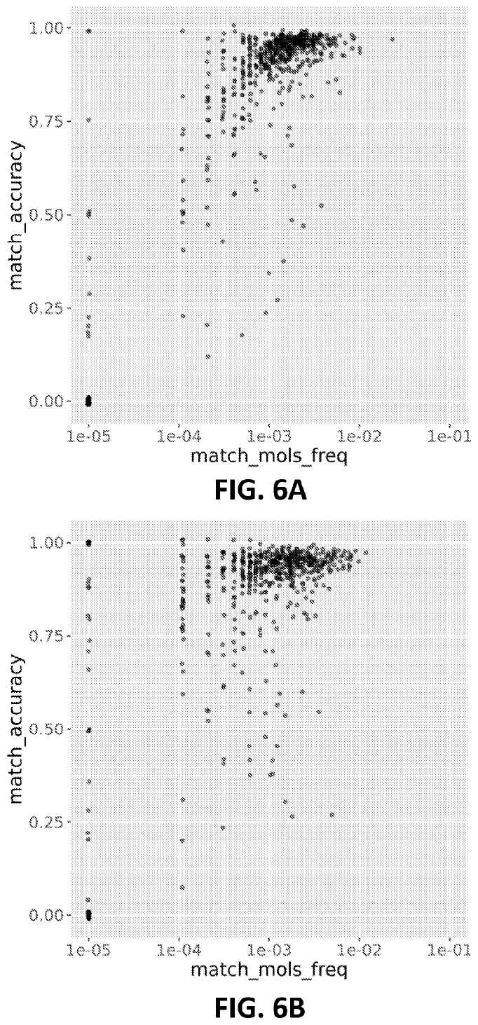

To assess the efficiency and accuracy of ligating C3Ja1ᵢ to the corresponding C3Ja2ᵢ (i=1 to 583), [GQ1} and [ACD} * were used to amplify the dsDNA pool 315 (result of R3.11), resulting in [GQ1|C3Jalᵢ|C3Ja2ᵢ|ACD}. Then Illumina sequencing adaptors along with unique molecule identifier (UMI) were added to flank the dsDNA [GQ1|C3Ja1ᵢ|C3Ja2ᵢ |ACD} and the resultant DNA library was analyzed by NGS. 538 out of the 583 of C3Ja1ᵢ (92%) were detected. For each detected C3Ja1ᵢ, two values were calculated: “match_mols_freq” and “match_accuracy.” The value “match_mols_freq” of a C3Ja1ᵢ (e.g., for a particular i) is defined by the UMI-corrected read numbers matched to the C3Ja1ᵢ divided by UMI-corrected read numbers matched to any C3Jal. Therefore, it reflects the frequency, or relative concentration of a [GQ1|C3Jalᵢ|C3Ja2; |ACD} (for a particular i) in the mixture. To calculate “match_accuracy”, all UMI-corrected reads mapped to a C3Ja1ᵢ are grouped and the sequences corresponding to the position of C3Ja2 in those reads were analyzed to determine if the correct C3Ja2 (e.g., C3Ja2ᵢ) was ligated to C3Ja1ᵢ. The fraction of UMI-corrected reads that mapped to C3Ja1ᵢ within this group was calculated and noted as “match_accuracy”. Therefore, it reflects the accuracy of the C3Ja1-C3Ja2 assembly. As can be seen in FIG. 6A, the vast majority of [GQ1|C3Jalᵢ|C3Ja2ᵢ|ACD} species have match_mols_freq values greater than 1e-4 (or 1×10⁻⁴). The term “uniform frequency” can be defined as 1 divided by the number of genes to be synthesized at the same time (e.g., the number of species in the oligo family, in this case 538 species). Uniform frequency is the ideal frequency if every species of the family has the same concentration. The results show that in the [GQ1|C3Jalᵢ|C3Ja2ᵢ|ACD} mixture, 513 of 583 (513/583=88%) have frequency (e.g., match_mols_freq values) higher than 0.1×[uniform frequency]. The median assembly accuracy (e.g., match_accuracy value) was 93.3%.

Similar analysis was done for pool 316 resulting from the C3Jb1-C3Jb2 assembly. The match_mols_freq and match_accuracy values for each species are shown on FIG. 6B. The results showed that 558 out of 583 (95.7%) species were detected and 497 of 583 (85%) have frequency higher than 0.1*[uniform frequency], with median assembly accuracy being 93.2%.

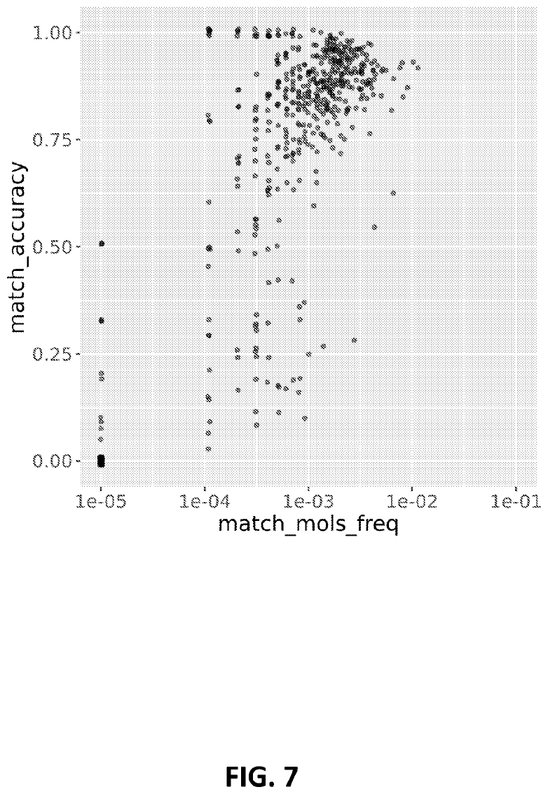

A similar strategy was used to characterize the pool 319, the Zip-guided assembly (reaction R3.15) product of 317 and 318. As shown on FIG. 7, the vast majority of species showed high frequency (the match_mols_freq values) and cumulative assembly accuracy (the match_accuracy values). Here, only UMI-corrected reads where all 4 product constituents (C3Ja1ᵢ, C3Ja2i, C3Jb1ᵢ, and C3Jb2ᵢ) are correct (matching to the same i) are considered correct reads. Specifically, 541 out of 583 (92.8%) species were detected, with 462 (76.5%) having frequency higher than 0.1×[uniform frequency]. The median cumulative accuracy among detected species was 77.8%.

Through steps R4.1 through R4.7, dsDNA pool 410 was prepared. Briefly, an adaptor removal reaction was carried out to remove [GQ1}:[GQ1*} (reaction R4.1) to form pool 402, which further underwent ssDNA generation to produce 403. The pool 404 containing ˜50 TRAV germline sequences, each having a 3′ single-stranded connector sequence (ConA#) was mixed with the pool 403, where each species of 403 was hybridized to the designated species in the pool 404 (reaction R4.3). Primer extension and ligation was carried out to produce pool 405. The [BCD}:[BCD*} domain contained a TypeIIS restriction site; cutting of 405 by the corresponding restriction enzyme (reaction R4.4) generated a 4-nt sticky end which was used to ligate a DNA segment (407) containing TRBC1 and a matching 4-nt sticky end (reaction R4.5). The resultant pool 408 was circularized (reaction R4.6) to form circular DNA pool 409, which was then linearized between GQ2 and GQ3 to from pool 410. The pool 410 underwent adaptor removal to remove [GQ3}:[GQ3*} (reaction R4.8 to form 411) and ssDNA generation (reaction R4.9 to form 413), before having each species in the pool 413 ligated to its corresponding TRBV germline sequences in pool 412 (reaction R4.10, analogous to R4.3), forming final product 414. It can be seen that in 410 and 414, each of [C3Jalᵢ|C3Ja2ᵢ} (e.g., [ConAᵢ|CDR3Jαᵢ}) and its corresponding [C3Jbli|C3Jb2;} (e.g., [ConBᵢ|CDR3Jβᵢ}) are joined without an intervening sequence that contains any Zip or any other variable sequences.

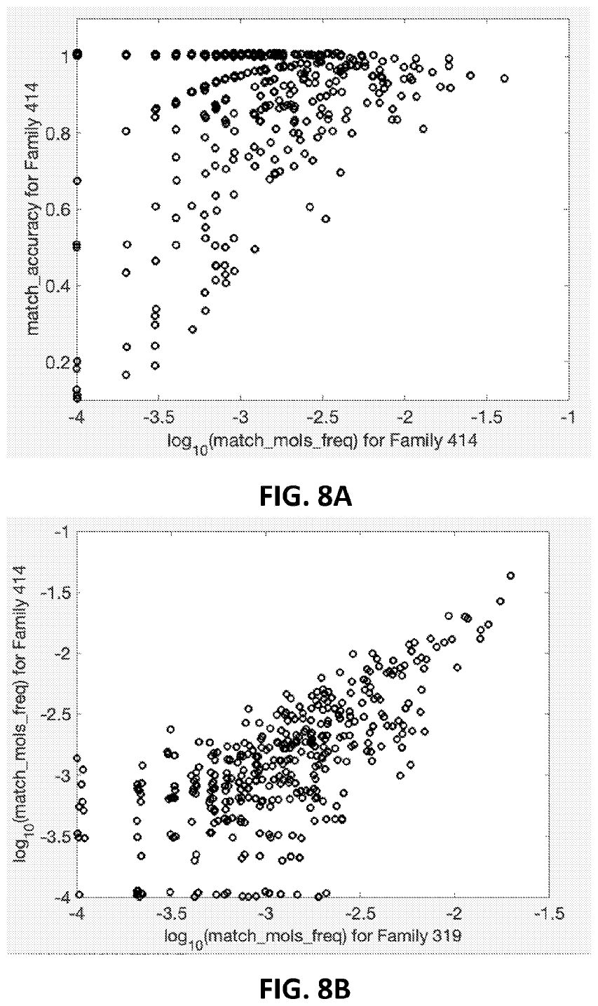

The final product 414 was characterized using an NGS-based method similar to that for 319 described above. The relative concentration and assembly accuracy of each species in 414 are shown in FIG. 8A. In this NGS-based analysis, only high-quality reads were retained. As a result, 384 out of 583 (66%) of the species were detected, and 358 (61%) showed frequency greater than 0.1×[uniform frequency]. The median assembly accuracy among detected species was 83.3%. Here, only when a molecule has TRAV, C3Ja1, C3Ja2, TRBV, C3Jb1, C3Jb2 all corresponding to the correct sequence it is regarded as a correctly assembled molecule. The fact that the accuracy (83.3%) is higher than the accuracy of the precursor (pool 319, 77.8%) is due to the fact that some incorrectly assembled molecules were not detected. Nevertheless, the relative concentration of each species in pools 414 and 319 is highly correlated (FIG. 8B).

### Example 4:3-Level Successive Zip-Based Assemblies to Form Genes Using 8 Families Oligonucleotide Pools

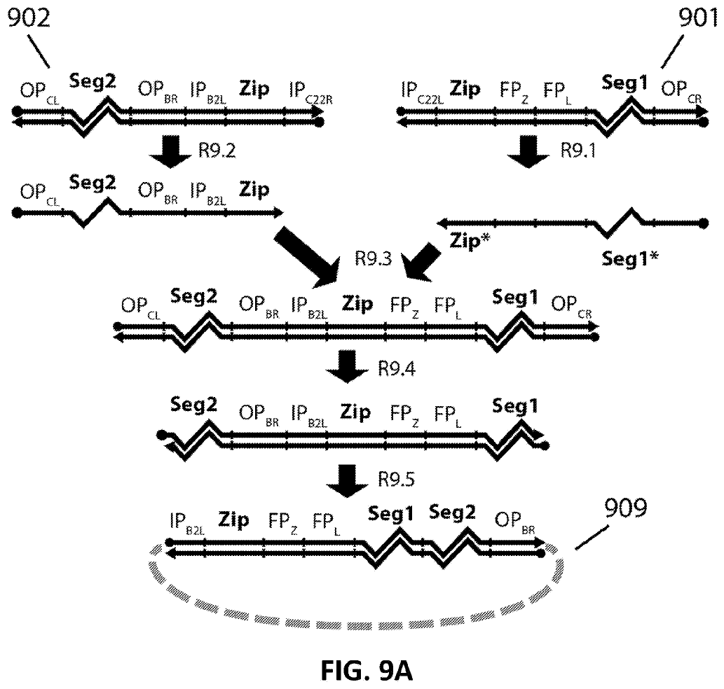

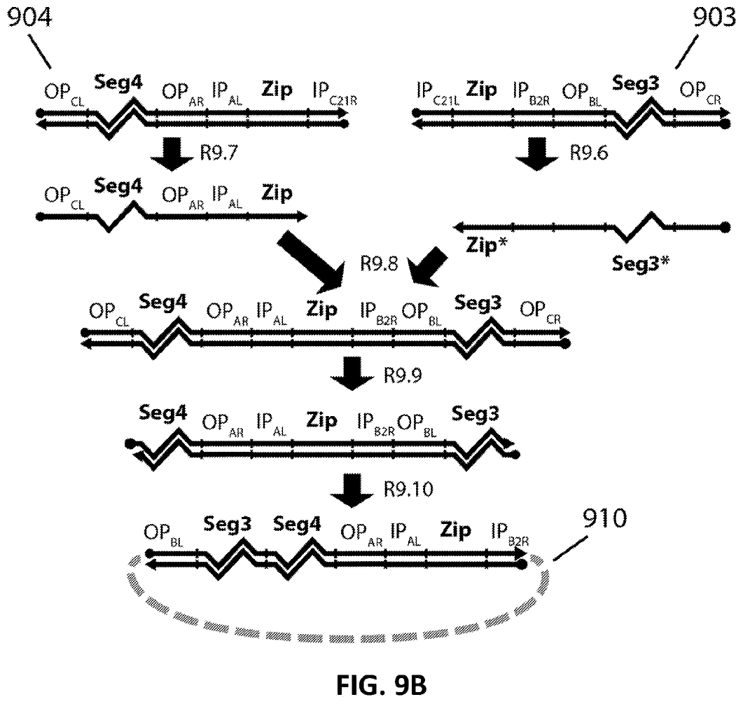

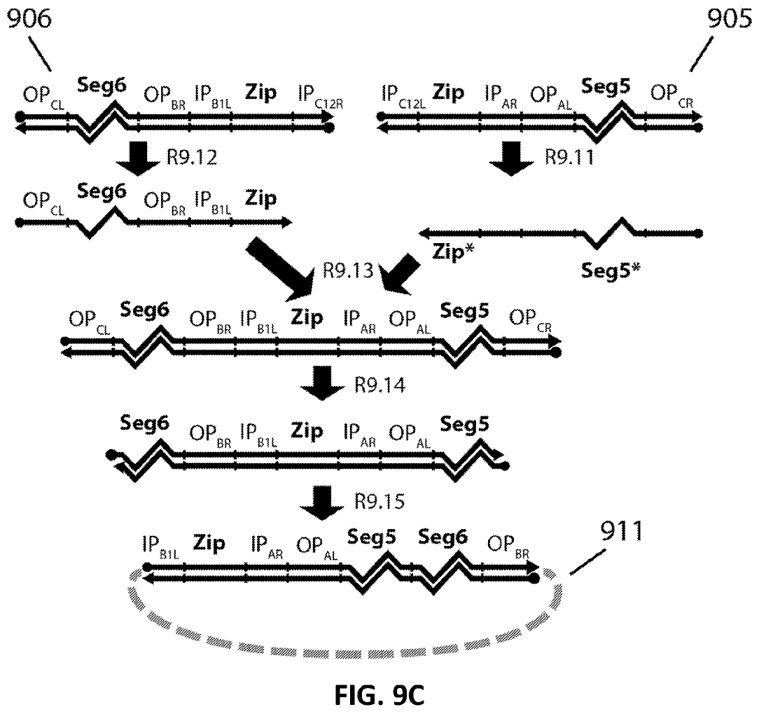

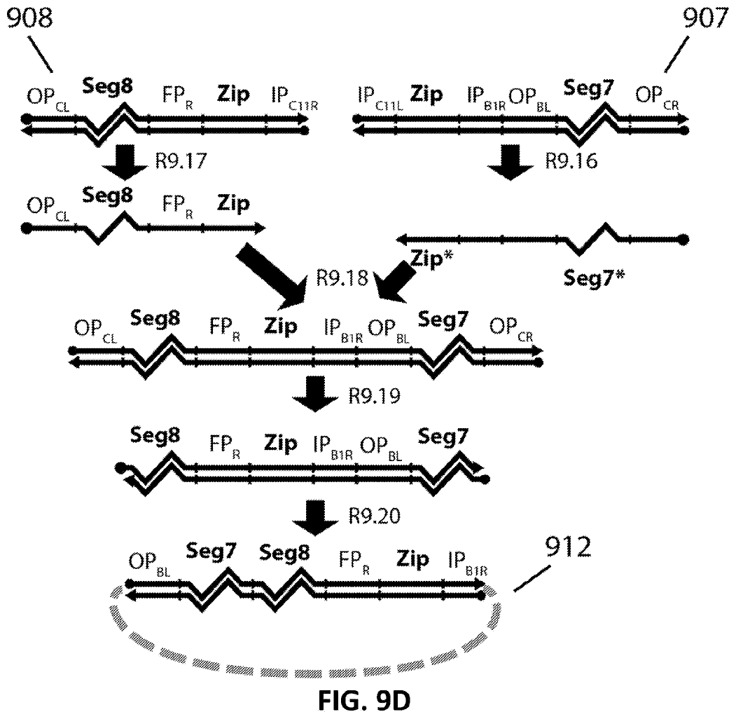

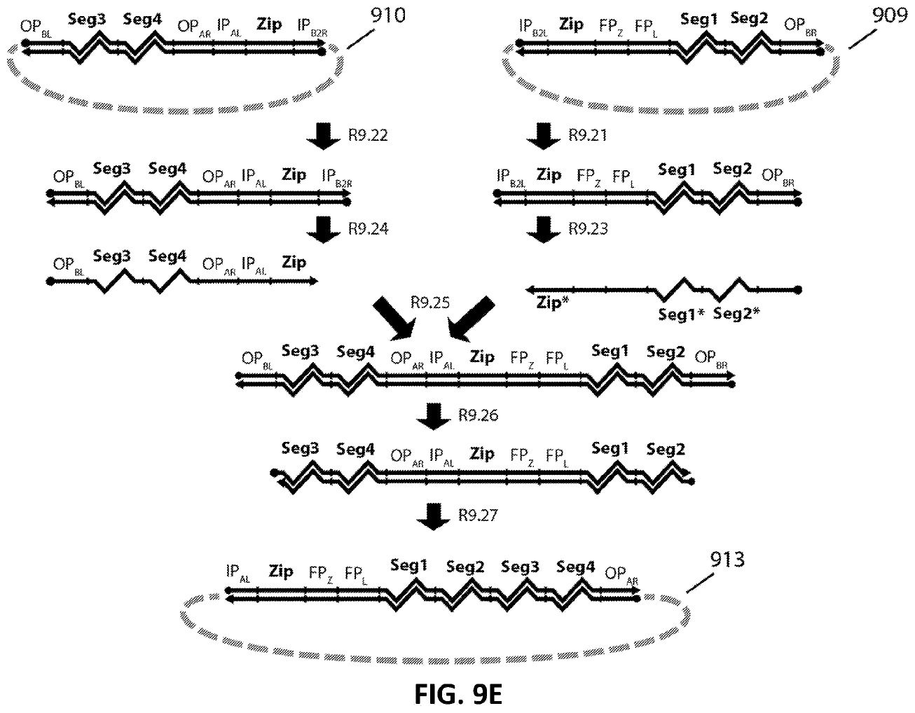

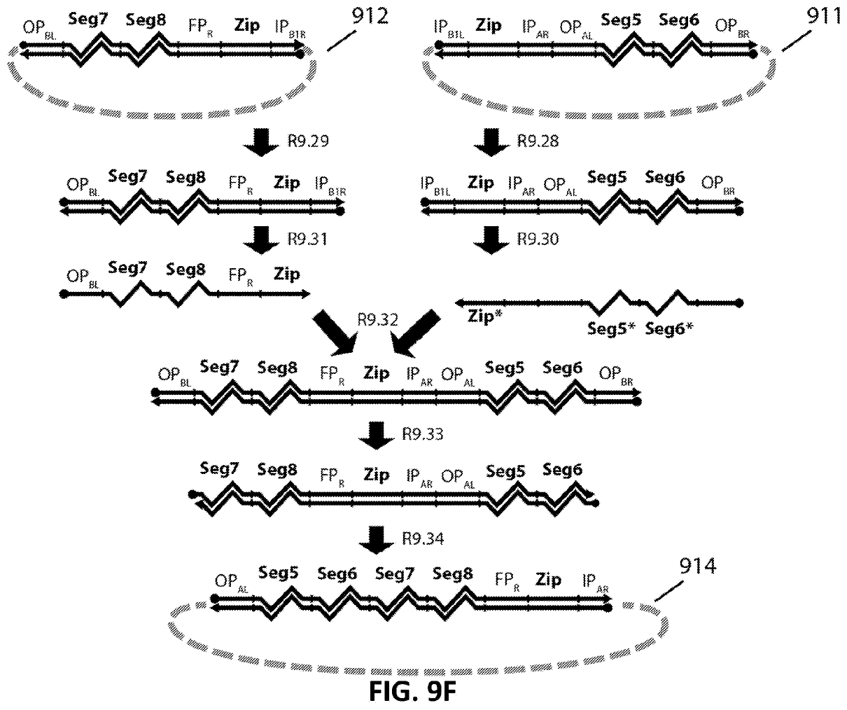

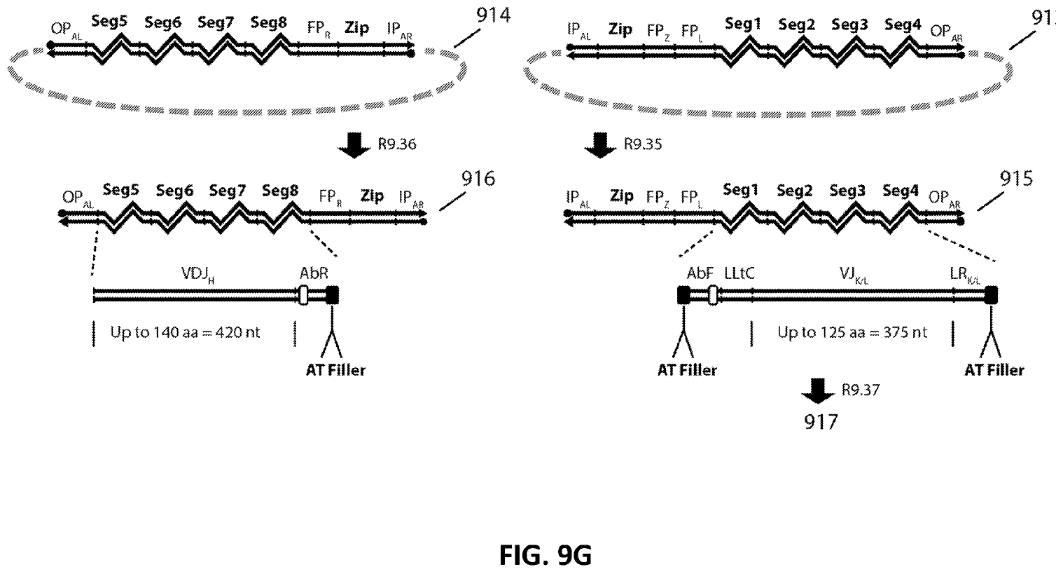

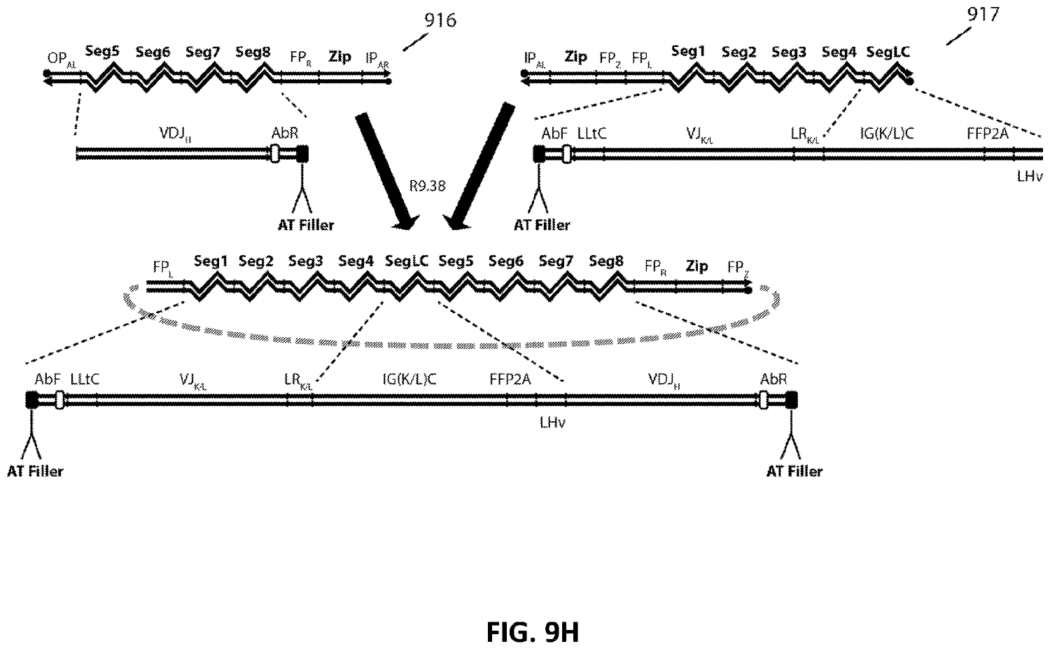

This example shows how a family of ˜1,000 genes, each containing ˜1.4-kb sequences (of which 1-kb were synthesized from oligo pool using the methods described herein) can be assembled through 3 levels of consecutive Zip-based assemblies, where the Zip sequences were reused at different levels of assembly reactions. An oligonucleotide pool containing 8 families of oligos (901, 902, 903, 904, 905, 906, 907 and 908, see FIGS. 9A-9D) was obtained from a commercial source. These oligo families have the following sequences described at domain level:
901: [IP_{C22L}|Zip|FP_{Z}|FP_{L|Seg}1|OP_{CR}}
902: [OP_{CL}|Seg2|OP_{BR}|IP_{B2L|Zip|IP}_{C22R}}
903: [IP_{C21L}|Zip|IP_{B2R}|OP_{BL|Seg}3|OP_{CR}}
904: [OP_{CL}|Seg4|OP_{AR}|IP_{AL|Zip|IP}_{C21R}}
905: [IP_{C12L}|Zip|IP_{AR|OP}_{AL|Seg}5|OP_{CR}}
906: [OP_{CL}|Seg6|OP_{BR|IP}_{B1L}|Zip|IP_{C12R}}
907: [IP_{C11L}|Zip|IP_{B1R|OP}_{BL|Seg}7|OP_{CR}}
908: [OP_{CL}|Seg8|FP_{R}|Zip|IP_{C11R}}

Among these domains, SegN (N=1 to 8) domains are Product Constituents, which along with Zip have species-specific sequences. All other domains are Operators and have common sequences. For example, [OPAL} on all oligos has the sequence 5′-AACACTGCTGAAGCTCCCAAT-3′, [OPBL} on all oligos has the sequence 5′-TCCCTGTTTGCCATTTCGCAT-3′. Other Operators have similar length and GC content.

First, eight PCRs were set up, each specifically amplifying a family from the initial oligonucleotide pool. Specifically:
dU-laden [IP_{C22L}} and 5′-protected [OP_{CR}*} were used to obtain pool 901.
5′-protected [OP_{CL}} and dU-laden [IP_{C22R}*} were used to obtain pool 902.
dU-laden [IP_{C21L}} and 5′-protected [OP_{CR}*} were used to obtain pool 903.
5′-protected [OP_{CL}} and dU-laden [IP_{C21R}*} were used to obtain pool 904.
dU-laden [IP_{C12L}} and 5′-protected [OP_{CR}*} were used to obtain pool 905.
5′-protected [OP_{CL}} and dU-laden [IP_{C12R}*} were used to obtain pool 906.
dU-laden [IP_{C11L}} and 5′-protected [OP_{CR}*} were used to obtain pool 907.
5′-protected [OP_{CL}} and dU-laden [IP_{C11R}*} were used to obtain pool 908.

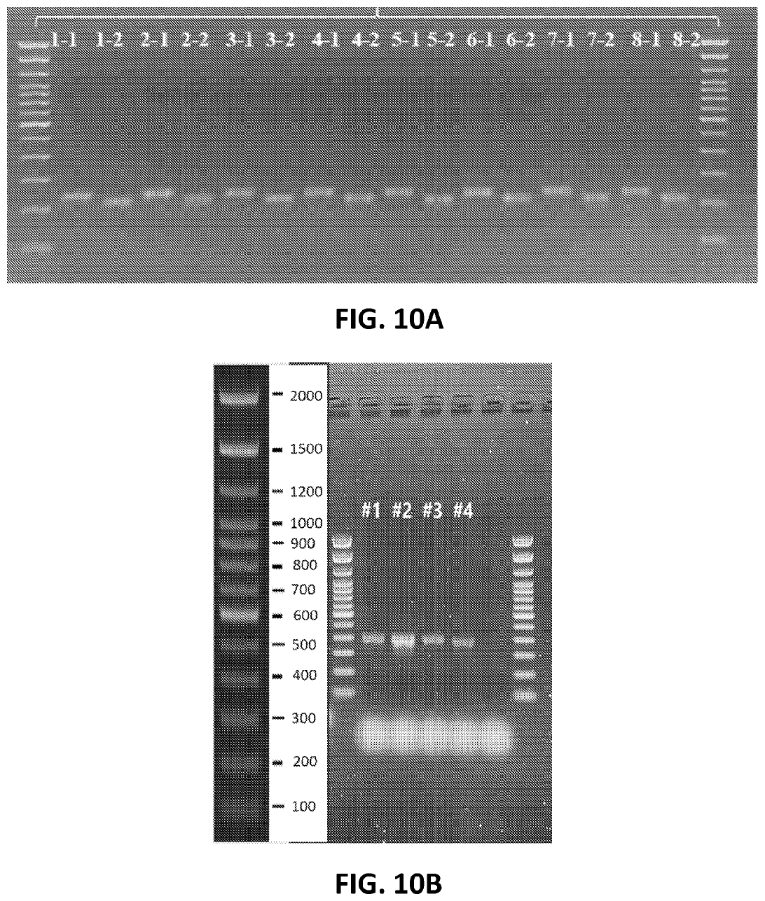

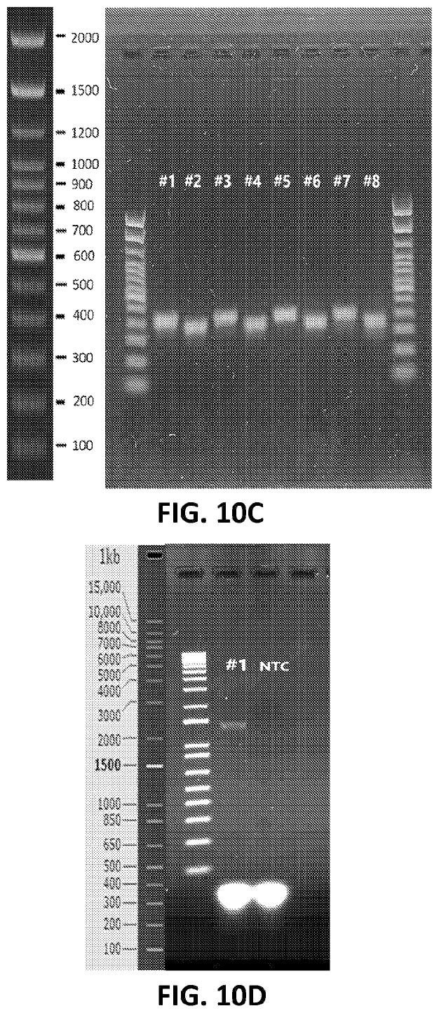

As shown in FIG. 9A, through adaptor removal reaction and ssDNA generation operated on 901 and 902 (reactions R9.1 and R9.2), these pools were converted to ssDNA with complementary Zip sequences. See FIG. 10A where lanes 1-1 and 2-1 show pools 901 and 902 before adaptor removal, respectively, and lanes 1-2 and 2-2 show pool 901 and 902 after adaptor removal, respectively. These two pools were allowed to hybridize through Zips, followed by primer extension (reaction R9.3), which generated a pool where the Product Constituents Seg1 and Seg2 from the same gene are linked through the Zip corresponding to the gene. This product was amplified with dU-laden [OP_{CL}} and [OP_{CR}}, underwent adaptor removal reactions (reaction R9.4), and was circularized through intramolecular blunt-end ligation (reaction R9.5) to form circular DNA pool 909, where the Product Constituents Seg1 and Seg2 are connected without intervening Zip.

FIG. 9B shows a similar series of reactions (reactions R9.6, R9.7, R9.8, R9.9 and R9.10) where pools 903 and 904 were converted to circular DNA pool 910, where Product Constituents Seg3 and Seg4 were ligated without intervening Zip.

FIG. 9C shows a similar series of reactions (reactions R9.11, R9.12, R9.13, R9.14 and R9.15) where pools 905 and 906 were converted to circular DNA pool 911, where Product Constituents Seg5 and Seg6 were ligated without intervening Zip.

FIG. 9D shows a similar series of reactions (reactions R9.16, R9.17, R9.18, R9.19 and R9.20) where pools 907 and 908 were converted to circular DNA pool 912, where Product Constituents Seg7 and Seg8 were ligated without intervening Zip.

The pools 903, 904, 905, 906, 907, and 908 prior to adaptor removal reaction are shown on lanes 3-1, 4-1, 5-1, 6-1, 7-1, and 8-1 of FIG. 10A.

The pools 903, 904, 905, 906, 907, and 908 after adaptor removal reaction are shown on lanes 3-2, 4-2, 5-2, 6-2, 7-2, and 8-2 of FIG. 10A.

The assembly reactions to form sequences [Seg1|Seg2}, [Seg3|Seg4}, [Seg5|Seg6}, and [Seg7|Seg8} (FIGS. 9A, 9B, 9C and 9D, respectively) are called level-1 assembly.

As shown in FIG. 9E, dU-laden [IP_{B2L}} and 5′-protected [OP_{BR}*} were used to amplify 909 (reaction R9.21, also see lane #1 of FIG. 10B for the PCR product). 5′-protected [OP_{BL}} and dU-laden [IP_{B2R}*} were used to amplify 910 (reaction R9.22, also see lane #2 of FIG. 10B for the PCR product). These PCR products underwent adaptor removal and ssDNA generation (reactions R9.23 and R.24) to form ssDNA pool with complementary Zip sequences. See FIG. 10C where lanes #1 and #3 show PCR products of R9.21 and R9.22 before adaptor removal, respectively, and lanes #2 and #4 show PCR products of R9.21 and R9.22 after adaptor removal, respectively. These two pools were allowed to hybridize through Zips, followed by primer extension (reaction R9.25) generated a pool where the Product Constituents [Seg1|Seg2} and [Seg3|Seg4} from the same gene were linked through the Zip corresponding to the gene. This product was amplified with dU-laden [OP_{BL}} and [OP_{BR}}, underwent adaptor removal reactions (reaction R9.26), and was circularized through intramolecular blunt-end ligation (reaction R9.27) to form circular DNA pool 913, where the Product Constituents [Seg1|Seg2} and [Seg3|Seg4} are connected without intervening Zip.

FIG. 9F shows a similar series of reactions (reactions R9.28, R9.29, R9.30, R9.31 and R9.32) where pools 911 and 912 were converted to circular DNA pool 914, where Product Constituents [Seg5|Seg6} and [Seg7|Seg8} were ligated without intervening Zip.

The PCR products of R9.28 and R9.29 prior to adaptor removal reaction are shown on lanes #5 and #7, respectively, of FIG. 10C.

The PCR products of R9.28 and R9.29 after adaptor removal reaction are shown on lanes #6 and #8, respectively, of FIG. 10C.

The assembly reactions to form sequences [Seg1|Seg2|Seg3|Seg4} and [Seg5|Seg6|Seg7|Seg8} (FIGS. 9E and 9F, respectively) are called level-2 assembly.

Next 5′-protected [OP_{AL}} and dU-laden [IP_{AR}*} were used to amplify circular dsDNA pool 914 into linear dsDNA pool 916 (reaction R9.36). [IP_{AL}} and [OP_{AR}*} were used to amplify circular dsDNA pool 913 into linear dsDNA pool 915 (reaction R9.35).

The genes to be synthesized in this example are antibody genes, where each of the Product Constituent [Seg1|Seg2|Seg3|Seg4} encodes an antibody light chain variable region, and each of the Product Constituent [Seg5|Seg6|Seg7|Seg8} encodes an antibody heavy chain variable region. Since the antibody chains for different genes have different lengths, stretches of scrambled filler sequences containing A and T bases (‘AT Filler’, black rounded squares in FIG. 9G) were padded upstream and downstream of the desired sequences within [Seg1|Seg2|Seg3|Seg4}, as well as downstream of the desired sequences within [Seg5|Seg6|Seg7|Seg8}, so that all molecules within the same family have the same lengths, resulting in sharp bands after electroporation (e.g., FIG. 10B and FIG. 10C). A common ˜20-mer [LR_{K}} sequence encoding the first ˜7 aa of the kappa light chain were added downstream of all light chain sequences containing a kappa J domain (i.e., VJ_{K}). Similarly, a common ˜20-mer [LR_{L}} sequence encoding the first ˜7 aa of the lambda light chain were added downstream of all light chain sequences containing a lambda J domain (i.e., VJ_{L}).

[IP_{AL}} and [LR_{K}*} were used to amplify all fragments within 915 that encode a kappa light chain. This product was ligated to kappa light chain constant domain (IGKC of 917, FIG. 9H) using standard technology.

[IP_{AL}} and [LR_{L}*} were used to amplify all fragments within 915 that encode a lambda light chain. This product was ligated to lambda light chain constant domain (IGLC of 917, FIG. 9H) using standard technology.

These two products were then mixed to form pool 917. Both light chain constant domains contained, at its 3′ end, a furin cleavage site, flexible linker, and P2A (FFP2A), followed by portion of the leader peptide of heavy chain (LHv, a common sequence for all genes). Next, dU-laden [IP_{AL}} and 5′-protected [LHv*} were used to amplify 917. This product was paired with pool 916, in Zip-based ligation and circularization reactions similar to those described before in this Example (collectively noted as R9.38), to form the final, ˜1.4 kb product (FIG. 10D), within which the sequences in [Seg1|Seg2|Seg3|Seg4} and [Seg5|Seg6|Seg7|Seg8} were synthesized de novo from oligo pools. AbF and AbR are common primer sequences that contain a restriction site (open rounded squares in FIGS. 9G and 9H) for further cloning purposes. The final assembly is called level-3 assembly.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBⱼ sequence of a jth polynucleotide when i≠j;
(c) contacting the first mixture and the second mixture, thereby generating a third mixture of at least n polynucleotides, wherein an ith polynucleotide comprises a SeqAᵢ sequence, a SeqBᵢ sequence and a ZipABᵢ sequence, wherein the ZipABᵢ sequence is different from a ZipABⱼ sequence of a jth polynucleotide when i≠j;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of jth polynucleotide when i≠j; and
(e) contacting the third mixture and the fourth mixture, thereby generating a fifth mixture of at least n polynucleotides, wherein an ith polynucleotide comprises the Seqᵢ sequence comprising the SeqAᵢ sequence, the SeqBᵢ sequence and the SeqCᵢ sequence.

2. The method of claim 1, wherein generating the third mixture of at least n polynucleotides comprises specifically linking, for each i, the ZipAᵢ sequence and the ZipBᵢ sequence.

3. The method of claim 1 or 2, wherein generating the fifth mixture of at least n polynucleotides comprises specifically linking, for each i, the ZipCᵢ sequence and the ZipABᵢ sequence.

4. A method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ (where j=1 to n) sequence of an jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBⱼ sequence of a jth polynucleotide when i≠j;
(c) generating a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABⱼ sequence of a jth polynucleotide when i≠j, and wherein generating comprises contacting the first mixture and the second mixture such that, for each i, the ZipAᵢ sequence specifically links to the ZipBᵢ sequence;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(e) generating a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein generating comprises contacting the third mixture and the fourth mixture such that, for each i, the ZipCᵢ sequence specifically links to the ZipABᵢ sequence.

5. The method of any one of claims 1-4, wherein, for each i, the ZipAᵢ sequence and the ZipBᵢ sequence are a same nucleic acid sequence or are complementary.

6. The method of any one of claims 1-5, wherein, for each i, the ZipABᵢ sequence is a same nucleic acid sequence as the ZipAᵢ sequence or the ZipBᵢ sequence.

7. The method of any one of claims 1-6, wherein for each i, the ZipAᵢ sequence and the ZipBᵢ sequence are different nucleic acid sequences.

8. The method of any one of claims 1-6, wherein for each i, the ZipABᵢ sequence, the ZipAᵢ sequence, and the ZipBᵢ sequence are a same nucleic acid sequence.

9. The method of any one of claims 1-5, wherein for each i, the ZipABᵢ sequence is a different nucleic acid sequence from the ZipAᵢ sequence or the ZipBᵢ sequence.

10. The method of claim 9, wherein the ZipABᵢ sequence, the ZipAᵢ sequence, and the ZipBᵢ sequence are different nucleic acid sequences.

11. A method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAᵢ (where j=1 to n) sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBᵢ sequence of a jth polynucleotide when i≠j;
(c) generating a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, wherein the ZipABᵢ sequence is a same nucleic acid sequence as the ZipA/sequence or the ZipBᵢ sequence, and wherein generating comprises contacting the first mixture and the second mixture such that, for each i, the ZipAᵢ sequence links to the ZipBᵢ sequence;
(d) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(e) generating a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein generating comprises contacting the third mixture and the fourth mixture such that, for each i, the ZipCᵢ sequence links to the ZipABᵢ sequence.

12. The method of claim 11, wherein, for each i, the ZipAᵢ sequence specifically links to the ZipBᵢ sequence.

13. The method of claim 11 or 12, wherein, for each i, the ZipCᵢ sequence specifically links to the ZipABᵢ sequence.

14. The method of any one of claims 11-13, wherein for each i, the ZipAᵢ sequence and the ZipBᵢ sequence are a same nucleic acid sequence or are complementary.

15. The method of any one of claims 11-14, wherein for each i, the ZipAᵢ sequence and the ZipBᵢ sequence are different nucleic acid sequences.

16. The method of any one of claims 11-14, wherein for each i, the ZipABᵢ sequence, the ZipAᵢ sequence, and the ZipBᵢ sequence are a same nucleic acid sequence.

17. The method of any one of claims 1-16, wherein the SeqAᵢ sequence, the SeqBᵢ sequence and the SeqCᵢ sequence of the Seqᵢ sequence are linked seamlessly without any intervening sequences.

18. The method of any one of claims 1-17, wherein the Seqᵢ sequence comprises the SeqAᵢ sequence and the SeqBᵢ sequence without an intervening sequence in between the SeqAᵢ sequence and the SeqBᵢ sequence.

19. The method of any one of claims 1-18, wherein the Seqᵢ sequence comprises the SeqBᵢ sequence and the SeqCᵢ sequence without an intervening sequence in between the SeqBᵢ sequence and the SeqCᵢ sequence.

20. The method of claim 18 or 19, wherein the Seqᵢ sequence with an intervening sequence in between the SeqAᵢ sequence and the SeqBᵢ sequence or the SeqBᵢ sequence and the SeqCᵢ sequence is not a functional genetic element.

21. The method of any one of claims 1-20, wherein the Seqᵢ sequence comprises the SeqAᵢ sequence, the SeqBᵢ sequence and the SeqCᵢ sequence sequentially from the 5′ end to the 3′ end.

22. The method of any one of claims 1-21, wherein for each i, the ZipAᵢ sequence and the ZipBᵢ sequence are connector sequences for specifically linking the SeqAᵢ sequence and the SeqBᵢ sequence.

23. The method of any one of claims 1-22, wherein for each i, the ZipCᵢ sequence and the ZipABᵢ sequence are connector sequences for specifically linking the SeqCᵢ sequence and a sequence comprising the SeqAᵢ sequence and the SeqBᵢ sequence.

24. The method of any one of claims 1-23, wherein the ZipCᵢ sequence and the ZipABᵢ sequence are a same nucleic acid sequence.

25. The method of any one of claims 1-23, wherein the ZipCᵢ sequence and the ZipABᵢ
sequence are complementary.

26. The method of any one of claims 1-23, wherein the ZipCᵢ sequence and the ZipABᵢ sequence are different nucleic acid sequences.

27. The method of any one of claims 1-26, wherein for each i, the ZipAᵢ sequence, the ZipBᵢ sequence, the ZipABᵢ sequence, or the ZipCᵢ sequence is from 5 nucleotides to 200 nucleotides in length.

28. The method of any one of claims 1-27, wherein for each i, the SeqAᵢ sequence, the SeqBᵢ sequence, or the SeqCᵢ sequence is from 5 nucleotides to 5,000 nucleotides in length.

29. The method of any one of claims 1-28, wherein, for each i, the ZipAᵢ sequence hybridizes to the ZipBᵢ sequence.

30. The method of claim 29, further comprising extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence.

31. The method of any one of claims 1-30, wherein for each i, the ith polynucleotide of the third mixture further comprises an Operator sequence that is a primer binding site.

32. The method of claim 31, wherein the Operator sequence is a same sequence among the third mixture of at least n polynucleotides.

33. The method of claim 31 or 32, further comprising removing the Operator sequence.

34. The method of claim 33, wherein removing comprises using an enzyme to degrade the Operator sequence.

35. The method of any one of claims 29-34, further comprising circularizing the ith polynucleotide comprising the SeqAᵢ sequence, the SeqBᵢ sequence and the ZipABᵢ sequence to generate a circularized polynucleotide.

36. The method of claim 35, wherein circularizing the ith polynucleotide comprising the SeqAᵢ sequence, the SeqBᵢ sequence and the ZipABᵢ sequence comprises circularizing the ith polynucleotide by a ligase.

37. The method of claim 35 or 36, further comprising linearizing the circularized polynucleotide.

38. The method of claim 37, wherein linearizing the circularized product comprises cutting the circularized polynucleotide or amplifying the circularized polynucleotide using polymerase chain reaction (PCR).

39. The method of claim 37 or 38, further comprising linearizing the circularized product such that the ZipABᵢ sequence is not flanked by the SeqAᵢ sequence and the SeqBᵢ sequence.

40. The method of any one of claims 37-39, further comprising exposing the ZipABᵢ sequence on a terminus of the ith polynucleotide comprising the SeqAᵢ sequence, the SeqBᵢ sequence and the ZipABᵢ sequence.

41. The method of any one of claims 1-40, wherein the ZipABᵢ sequence is not flanked by the SeqAᵢ sequence and the SeqBᵢ sequence.

42. The method of claim 41, wherein the ZipABᵢ sequence is at a terminus of the ith polynucleotide comprises the SeqAᵢ sequence, the SeqBᵢ sequence and the ZipABᵢ sequence.

43. The method of any one of claims 1-42, wherein the ZipCᵢ sequence hybridizes to the ZipABᵢ sequence.

44. The method of claim 43, further comprising repeating operations of claims 30-40 for the third mixture of at least n polynucleotides and the fourth mixture of at least n polynucleotides, thereby generating the fifth mixture of at least n polynucleotides.

45. The method of claim 43 or 44, further comprising removing the ZipCᵢ sequence and the ZipABᵢ sequence, thereby generating the ith polynucleotide comprises the Seqᵢ sequence comprising the SeqAᵢ sequence, the SeqBᵢ sequence and the SeqCᵢ sequence.

46. The method of any one of claims 1-45, further comprising, prior to (a) or (b), providing a pool of polynucleotides comprising the at least n polynucleotides of the first mixture, the at least n polynucleotides of the second mixture, and/or the at least n polynucleotides of the fourth mixture.

47. The method of claim 46, further comprising amplifying the at least n polynucleotides of the first mixture, the at least n polynucleotides of the second mixture, and/or the at least n polynucleotides of the fourth mixture from the pool to generate double-stranded polynucleotides.

48. The method of claim 47, wherein only the at least n polynucleotides of the first mixture, the at least n polynucleotides of the second mixture, or the at least n polynucleotides of the fourth mixture are amplified from the pool.

49. The method of any one of claims 46-48, further comprising removing an Operator sequence from the double-stranded polynucleotides, and wherein the Operator sequence is a primer binding site.

50. The method of any one of claims 47-49, wherein degrading one strand of the double-stranded polynucleotides to generate the at least n polynucleotides of the first mixture, the at least n polynucleotides of the second mixture, and/or the at least n polynucleotides of the fourth mixture.

51. A method of synthesizing a plurality of n different polynucleotides (where n is equal to or greater than 2) in a same mixture, wherein
(i) an ith polynucleotide of the plurality comprises a Seqisequence (where i=1 to n), and
(ii) the Seqi sequence comprises a SeqAi sequence, a SeqBi sequence and a SeqCᵢ sequence, the method comprising:
(a) providing a mixture comprising a first subpopulation of at least n polynucleotides, a second subpopulation of at least n polynucleotides, and a third subpopulation of at least n polynucleotides, wherein
(1) in the first subpopulation, an ith polynucleotide comprises a SeqAᵢ sequence and a ZipAᵢ sequence, and wherein the ZipAᵢ sequence is different from a ZipAᵢ sequence of a jth polynucleotide when i≠j,
(2) in the second subpopulation, an ith polynucleotide comprises a SeqBᵢ sequence and a ZipBᵢ sequence, and wherein the ZipBᵢ sequence is different from a ZipBⱼ sequence of a jth polynucleotide when i≠j, and
(3) in the third subpopulation, an ith polynucleotide comprises a SeqCᵢ sequence and a ZipCᵢ sequence, and wherein the ZipCᵢ sequence is different from a ZipCⱼ sequence of a jth polynucleotide when i≠j; and
(b) generating a plurality of n polynucleotides, wherein an ith polynucleotide of the plurality comprises the Seqᵢ sequence comprising the SeqAᵢ sequence, the SeqBᵢ sequence and the SeqCᵢ sequence, wherein generating comprises specifically linking the SeqAᵢ sequence, the SeqBᵢ sequence and the SeqCᵢ sequence.

52. The method of claim 51, wherein in the second subpopulation, the ZipBᵢ sequence is a ZipB1ᵢ sequence, and the ith polynucleotide further comprises a ZipB2ᵢ sequence.

53. The method of claim 52, wherein the SeqBᵢ sequence is located in between the ZipB1ᵢ sequence and the ZipB2ᵢ sequence.

54. The method of claim 52, wherein the ZipB1ᵢ sequence is located in between the SeqBᵢ sequence and the ZipB2ᵢ sequence.

55. The method of claim 52, wherein the ZipB2ᵢ sequence is located in between the SeqBᵢ sequence and the ZipB1ᵢ sequence.

56. The method of any one of claims 51-55, wherein the SeqA sequence, the SeqBᵢ sequence and the SeqCᵢ sequence of the Seqᵢ sequence are linked seamlessly without any intervening sequences.

57. The method of any one of claims 51-56, wherein the Seqᵢ sequence comprises the SeqAᵢ sequence and the SeqBᵢ sequence without an intervening sequence in between the SeqAᵢ sequence and the SeqBᵢ sequence.

58. The method of any one of claims 51-57, wherein the Seqᵢ sequence comprises the SeqBᵢ sequence and the SeqCᵢ sequence without an intervening sequence in between the SeqBᵢ sequence and the SeqCᵢ sequence.

59. The method of any one of claims 51-58, wherein the Seqᵢ sequence comprises the SeqAᵢ sequence, the SeqBᵢ sequence and the SeqCᵢ sequence sequentially from the 5′ end to the 3′ end.

60. The method of any one of claims 51-59, wherein the ZipAⱼ sequence and the ZipBᵢ sequence are connector sequences for specifically linking the SeqAᵢ sequence and the SeqBᵢ sequence.

61. The method of any one of claims 52-60, wherein the ZipB2ᵢ sequence and the ZipCᵢ sequence are connector sequences for specifically linking the SeqBᵢ sequence and the SeqCᵢ sequence.

62. The method of any one of claims 51-61, wherein for each i, the ZipAᵢ sequence and the ZipBᵢ sequence are a same nucleic acid sequence.

63. The method of any one of claims 51-61, wherein for each i, the ZipAᵢ sequence and the ZipBᵢ sequence are complementary.

64. The method of any one of claims 51-61, wherein for each i, the ZipAᵢ sequence and the ZipBᵢ sequence are different nucleic acid sequences.

65. The method of any one of claims 52-64, wherein for each i, the ZipB2ᵢ sequence and the ZipCᵢ sequence are a same nucleic acid sequence.

66. The method of any one of claims 52-64, wherein for each i, the ZipB2ᵢ sequence and the ZipCᵢ sequence are complementary.

67. The method of any one of claims 52-64, wherein for each i, the ZipB2ᵢ sequence and the ZipCᵢ sequence are different nucleic acid sequences.

68. The method of any one of claims 51-67, wherein for each i, the ZipAᵢ sequence, the ZipBᵢ sequence, the ZipB1ᵢ sequence, the ZipB2ᵢ sequence, or the ZipCᵢ sequence is from 5 nucleotides to 200 nucleotides in length.

69. The method of any one of claims 51-68, wherein for each i, the SeqAᵢ sequence, the SeqBᵢ sequence, or the SeqCᵢ sequence is from 5 nucleotides to 5,000 nucleotides in length.

70. The method of any one of claims 52-69, further comprising specifically linking (i) the ZipAᵢ sequence and the ZipB1ᵢ sequence, and/or (ii) the ZipB2ᵢ sequence and the ZipCᵢ sequence.

71. The method of claim 70, wherein linking comprising hybridizing (i) the ZipAᵢ sequence and the ZipB1ᵢ sequence, and/or (ii) the ZipB2ᵢ sequence and the ZipCᵢ sequence.

72. The method of claim 70 or 71, further comprising generating a plurality of intermediate products, wherein an ith intermediate product of the plurality comprises the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence.

73. The method of claim 72, wherein the ith intermediate product of the plurality comprises the SeqAᵢ sequence, the ZipAᵢ sequence (or the ZipB1ᵢ sequence), the SeqBᵢ sequence, the ZipCᵢ sequence (or the ZipB2ᵢ sequence) and the SeqCᵢ sequence sequentially from 5′ end to 3′ end.

74. The method of claim 72 or 73, further comprising removing the ZipAᵢ sequence (or the ZipB1ᵢ sequence) and the ZipCᵢ sequence (or the ZipB2ᵢ sequence), thereby generating the Seqᵢ sequence comprising the SeqAᵢ sequence, the SeqBᵢ sequence and the SeqCᵢ sequence without any intervening sequence.

75. The method of claim 74, wherein removing comprises using a DNA tweezer.

76. The method of claim 75, wherein using the DNA tweezer comprises degrading one strand of the ZipAᵢ sequence or the ZipCᵢ sequence region, and using a staple strand to hybridize with regions flanking the ZipAᵢ sequence or the ZipCᵢ sequence on the complementary strand to bring the SeqAᵢ sequence, the SeqBᵢ sequence and the SeqCᵢ sequence region in close proximity for ligation.

77. The method of any one of claims 1-76, wherein concatenation of the SeqAᵢ sequence and the SeqBᵢ sequence without an intervening sequence is a functional genetic element.

78. The method of claim 77, wherein concatenation of the SeqAᵢ sequence, the SeqBᵢ sequence and the SeqCᵢ sequence without any intervening sequence is a functional genetic element.

79. The method of claim 77 or 78, wherein the functional genetic element is a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, or a primer-extension gRNA for prime editing.

80. The method of any one of claims 77-79, wherein the functional genetic element does not comprise a sequence that is identical to the ZipAᵢ sequence, the ZipBᵢ sequence, the ZipABⱼ sequence or the ZipCᵢ sequence.

81. The method of any one of claims 1-80, wherein a plurality of at least 2, 10, 20, 30, 40, 50, 60, 70, 80, 90, 100, 500, 1,000, 5,000 or more different polynucleotides are synthesized.

82. The method of any one of claims 1-81, wherein each polynucleotide of the plurality synthesized is from about 15 to about 15,000 nucleotides in length.

83. A method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence and a SeqBᵢ sequence, the method comprising:
(a) providing a first mixture of at least n polynucleotides, wherein an ith polynucleotide comprises a SeqAᵢ sequence and a ZipAᵢ sequence, and wherein the ZipAᵢ sequence is different from a ZipAⱼ sequence of a jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBⱼ sequence of a jth polynucleotide when i≠j; and
(c) contacting the first mixture and the second mixture, thereby generating a third mixture of a plurality of n polynucleotides, wherein an ith polynucleotide comprises a ZipABᵢ sequence, a SeqAᵢ sequence and a SeqBᵢ sequence sequentially from 5′ end to 3′ end, and wherein the ZipABᵢ sequence is different from a ZipABᵢ sequence of a jth polynucleotide when i≠j;
wherein the SeqAᵢ sequence and the SeqBᵢ sequence are linked without an intervening sequence.

84. The method of claim 83, wherein for each i, the ZipAᵢ sequence and the ZipBᵢ
sequence are connector sequences for specifically linking the SeqAᵢ sequence and the SeqBᵢ sequence.

85. The method of claim 83 or 84, wherein generating the third mixture in (c) comprises specifically linking, for each i, the ZipAᵢ sequence and the ZipBᵢ sequence.

86. The method of any one of claims 83-85, wherein linking comprising hybridizing, for each i, the ZipAᵢ sequence and the ZipBᵢ sequence.

87. The method of claim 85 or 86, further comprising extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence.

88. The method of any one of claims 83-87, further comprising generating an intermediate product comprising the SeqBᵢ sequence, the ZipABᵢ sequence and the SeqAᵢ sequence sequentially from 5′ end to 3′ end.

89. The method of any one of claims 83-88, further comprising contacting the third mixture with a fourth mixture of at least n polynucleotides, wherein an ith polynucleotide of the fourth mixture comprises a SeqCᵢ sequence and a ZipCᵢ sequence, and wherein the ZipCᵢ sequence is different from a ZipCⱼ sequence when i≠j.

90. The method of claim 89, further comprising generating a fifth mixture of at least n polynucleotides, wherein an ith polynucleotide comprises the Seqᵢ sequence comprising the SeqAᵢ sequence and the SeqBᵢ sequence and further comprising the SeqCᵢ sequence.

91. The method of claim 90, wherein the SeqBᵢ sequence and the SeqCᵢ sequence are linked without an intervening sequence.

92. A method of synthesizing a plurality of n different polynucleotides (where n is equal to or greater than 2) in a mixture, wherein
(i) an ith polynucleotide of the plurality comprises a Seqᵢ sequence (where i=1 to n), and
(ii) the Seqᵢ sequence comprises a SeqAᵢ sequence, a SeqBᵢ sequence and a SeqCᵢ sequence, the method comprising:
(a) providing a first mixture of at least n polynucleotides, wherein an ith polynucleotide comprises a SeqAᵢ sequence and a ZipAᵢ sequence, and wherein the ZipA sequence is different from a ZipAⱼ (where j=1 to n) sequence of an jth polynucleotide when i≠j;
(b) providing a second mixture of at least n polynucleotides, wherein an ith polynucleotide comprises a SeqBᵢ sequence and a ZipBᵢ sequence, and wherein the ZipBᵢ sequence is different from a ZipBⱼ sequence of a jth polynucleotide when i≠j;
(c) contacting the first mixture and the second mixture to generate a third mixture of at least n polynucleotides, an ith polynucleotide of the third mixture comprising a SeqAᵢ sequence, a SeqBᵢ sequence and a ZipABᵢ sequence, wherein the ZipABᵢ sequence is different from a ZipABᵢ sequence of a jth polynucleotide when i≠j, and wherein, for each i, the ZipAᵢ sequence specifically links to the ZipBᵢ sequence;
(d) optionally, within the third mixture, for each i, extending a free 3′ end of the ZipAᵢ sequence or the ZipBᵢ sequence using the ith polynucleotide from the first mixture or the second mixture as a template to generate the ith polynucleotide comprising the SeqAᵢ sequence, the SeqBᵢ sequence and the ZipABᵢ sequence;
(e) optionally, within the third mixture, removing a sequence segment from 3′ and/or 5′end of the ith polynucleotide;
(f) optionally, within the third mixture, circularizing the ith polynucleotide comprising the SeqA sequence, the SeqBᵢ sequence and the ZipABᵢ sequence to generate a circularized polynucleotide;
(g) optionally, within the third mixture, linearizing the circularized polynucleotide such that, for each i, the ZipABᵢ sequence is not flanked by the SeqAᵢ sequence and the SeqBᵢ sequence;
(h) providing a fourth mixture of at least n polynucleotides, wherein an ith polynucleotide comprises a SeqCᵢ sequence and a ZipCᵢ sequence, and wherein the ZipCᵢ sequence is different from a ZipCⱼ sequence of a jth polynucleotide when i≠j; and
(i) contacting the third mixture and the fourth mixture to generate a fifth mixture of at least n polynucleotides, an ith polynucleotide of the fifth mixture comprising the Seqᵢ sequence comprising the SeqAᵢ sequence, the SeqBᵢ sequence and the SeqCᵢ sequence, wherein, for each i, the ZipCᵢ sequence specifically links to the ZipABᵢ sequence.

93. The method of claim 92, wherein for each i, the ZipAᵢ sequence and the ZipBᵢ sequence are a same nucleic acid sequence.

94. The method of claim 92 or 93, wherein for each i, the ZipAᵢ sequence and the ZipBᵢ sequence are complementary.

95. The method of any one of claims 92-94, wherein for each i, the ZipABᵢ sequence is a same nucleic acid sequence as the ZipAᵢ sequence or the ZipBᵢ sequence.

96. The method of any one of claims 92-95, wherein the SeqAᵢ sequence, the SeqBᵢ sequence and the SeqCᵢ sequence are specifically linked without any intervening sequences.

97. The method of any one of claims 92-96, wherein concatenation of the SeqAᵢ sequence, the SeqBᵢ sequence and the SeqCᵢ sequence without any intervening sequence is a functional genetic element.

98. A composition comprising the first mixture, the second mixture, or the third mixture of any one of claims 1-50 and 83-97, or the mixture of any one of claims 51-82.

99. A composition for synthesizing a plurality of n different polynucleotides, comprising:
a first mixture of at least n polynucleotides, wherein an ith polynucleotide comprises a SeqAᵢ sequence and a ZipAᵢ sequence (where i=1 ton), and wherein the ZipAᵢ sequence is different from a ZipAᵢ sequence of a jth polynucleotide when i≠j; and
a second mixture of at least n polynucleotides, wherein an ith polynucleotide comprises a SeqBᵢ sequence and ZipBᵢ sequence (where i=1 to n), and wherein the ZipBᵢ sequence is different from a ZipBᵢ sequence of a jth polynucleotide when i≠j; wherein, for each of i,
concatenation of the SeqAᵢ sequence and the SeqBᵢ sequence without intervening sequence is a functional genetic element, and
the ZipAᵢ sequence and the ZipBᵢ sequence are connector sequences for linking the SeqAj sequence and the SeqBᵢ sequence.

100. The composition of claim 99, wherein the first mixture and the second mixture are within a same compartment or a same mixture.

101. The composition of claim 99 or 100, wherein the ZipAᵢ sequence and the ZipBᵢ sequence are specifically linked.

102. The composition of claim 101, wherein the ZipAᵢ sequence and the ZipBᵢ sequence are hybridized.

103. The composition of any one of claims 99-102, wherein the ZipAᵢ sequence and the ZipBᵢ sequence are a same nucleic acid sequence, complementary nucleic acid sequences, or different nucleic acid sequences.

104. The composition of any one of claims 99-103, further comprising a third mixture of at least n polynucleotides, wherein an ith polynucleotide comprises a SeqCᵢ sequence and a ZipCᵢ sequence, and wherein the ZipCᵢ sequence is different from a ZipCᵢ sequence of a jth polynucleotide when i≠j.

105. The composition of claim 104, wherein for each of i, concatenation of the SeqAᵢ sequence, the SeqBᵢ sequence, and the SeqCᵢ sequence without any intervening sequence is a functional genetic element.

106. The composition of claim 104 or 105, wherein the ZipCᵢ sequence is a connector sequence for linking the SeqCᵢ sequence and a sequence comprising the SeqAᵢ sequence and the SeqBᵢ sequence.

107. The composition of any one of claims 104-106, wherein the ZipCᵢ sequence is a same nucleic acid sequence as the ZipAᵢ sequence or the ZipBᵢ sequence.

108. The composition of any one of claims 104-106, wherein the ZipCⱼ sequence is a different nucleic acid sequence from the ZipAᵢ sequence or the ZipBᵢ sequence.

109. The composition of any one of claims 104-106, wherein the ZipCᵢ sequence, the ZipAᵢ sequence and the ZipBᵢ sequence are a same nucleic acid sequence.

110. The composition of any one of claims 99-109, wherein the first mixture, the second mixture and the third mixture are within a same compartment or a same mixture.

111. The composition of any one of claims 105-110, wherein the functional genetic element comprises a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, and/or a primer-extension gRNA for prime editing.

112. A composition comprising a polynucleotide having at least two double-stranded regions separated by a single-stranded region, wherein the at least two double-stranded regions comprise a first double-stranded region and a second double-stranded region, wherein the single-stranded region hybridizes with a stable strand such that the single-stranded region forms a loop stabilized by the stable strand to bring a 3′ end of the first double-stranded region and a 5′ end of the second double-stranded region to close proximity, and wherein the first double-stranded region and the second double-stranded region are from a same functional genetic element.

113. The composition of claim 112, wherein the functional genetic element is a gene, a protein-coding sequence, a promoter, an internal ribosome entry site, a ribozyme, an aptamer, a nucleic acid sequence that is capable of being specifically bound by a transposase, a guide ribonucleic acid (gRNA) for CRISPR/Cas9 based gene editing, or a primer-extension gRNA for prime editing.

114. The composition of claim 112 or 113, wherein the 3′ end of the first double-stranded region and the 5′ end of the second double-stranded region are in close proximity for ligation.

115. The composition of any one of claims 112-114, wherein the 3′ end of the first double-stranded region and the 5′ end of the second double-stranded region are joined.

116. The composition of any one of claims 112-115, wherein the 3′ end of the first double-stranded region and the 5′ end of the second double-stranded region are ligated.

117. The composition of any one of claims 112-116, wherein the single-stranded region comprises, from 5′ to 3′, a first segment and a second segment, and the stable strand comprises, from 5′ to 3′, a third segment and a fourth segment, and wherein the first segment hybridizes with the third segment and the second segment hybridizes with the fourth segment.

118. The composition of any one of claims 112-117, further comprising a plurality of polynucleotides, each having at least two double-stranded regions separated by a single-stranded region, wherein the at least two double-stranded regions comprise a first double-stranded region and a second double-stranded region, wherein the single-stranded region hybridizes with a stable strand such that the single-stranded region forms a loop stabilized by the stable strand to bring a 3′ end of the first double-stranded region and a 5′ end of the second double-stranded region to close proximity, and wherein the first double-stranded region and the second double-stranded region are from a same functional genetic element.

119. The composition of claim 118, wherein each polynucleotide of the plurality is a different functional genetic element.

120. The composition of any one of claims 112-119, wherein the polynucleotide comprises three double-stranded regions separated by two single-stranded regions, each single-stranded region hybridizing with a stable strand.

121. The composition of claim 120, wherein the three double-stranded regions are from a same functional genetic element.
